(19) United States
(12) Patent Application Publication
(10) Pub. No.: US 2024/0124400 A1
(43) Pub. Date: Apr. 18, 2024

(54) LPXH TARGETING COMPOUNDS, COMPOSITIONS THEREOF, AND METHODS OF MAKING AND USING THE SAME

(71) Applicant: DUKE UNIVERSITY, Durham, NC (US)

(72) Inventors: Pei Zhou, Durham, NC (US); Jiyong Hong, Durham, NC (US)

(73) Assignee: DUKE UNIVERSITY, Durham, NC (US)

(21) Appl. No.: 17/768,152

(22) PCT Filed: Oct. 12, 2020
(86) PCT No.: PCT/US20/55243

§ 371 Date: Apr. 11, 2022

Related U.S. Application Data
(60) Provisional application No. 62/913,784, filed on Oct. 11, 2019.

(51) Int. Cl. C07D 209/30; C07C 311/20; C07H 11/04

(52) U.S. Cl. CPC C07D 209/30; C07C 311/20; C07H 11/04

(57) ABSTRACT

LpxH targeting compounds, compositions thereof, as well as methods for for making and using the same are disclosed herein. The LpxH target compounds typically have a structure pursuant to Formula (I) and/or a salt thereof, wherein R_{b} is selected from a single bond, C₄ to C₁₀ unsubstituted aryl, C₄ to C₁₀ substituted aryl, unsubstituted or substituted four to ten member heterocycle ring, C₁ to C₁₀ unsubstituted alkyl, and C₁ to C₁₀ substituted alkyl; R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted aryl, C₄ to C₁₀ substituted aryl, unsubstituted or substituted four to ten member heterocycle ring, C₁ to C₁₀ unsubstituted alkyl, or C₁ to C₁₀ substituted alkyl; and R_{d} and Rₑ are independently hydrogen, —OH, —COH, —COH, —COC, —COOH, R_{f}, or are taken together as an unsubstituted or substituted four to eight member nitrogen containing heterocycle ring.

### CROSS-REFERENCE TO RELATED APPLICATIONS

This application claims priority to U.S. Provisional Patent Application No. 62/913,784, filed on Oct. 11, 2019, entitled, “LPXH-TARGETING ANTIBIOTICS AND METHODS OF MAKING AND USING SAME,” the entirety of which is herein incorporated by reference for all purposes.

### ACKNOWLEDGEMENT OF GOVERNMENT SUPPORT

The present invention was made with Government support under U.S. Federal Grant No. GM115355, awarded by the National Institutes of Health/National Institute of General Medical Sciences (NIH/NIGMS), and U.S. Federal Grant No. A1139216, awarded by the National Institute of Allergy and Infectious Disease (NIAID). The U.S. Federal Government has certain rights to this invention.

### FIELD OF THE DISCLOSURE

Aspects of the instant disclosure relate to LpxH targeting compounds and compositions thereof. Additional aspects relate to methods for making and using such LpxH targeting compounds and compositions thereof for treating and/or ameliorating the symptoms of patients with a Gram-negative bacteria.

### BACKGROUND OF THE DISCLOSURE

The emergence of multi- and pan-drug resistant nosocomial Gram-negative pathogens has become a major public health threat by significantly increasing patient morbidity and mortality as well as healthcare cost, prompting the World Health Organization (WHO) to declare a list of priority Gram-negative bacteria for accelerated development of novel antimicrobial therapeutics.

Gram-negative bacteria are characterized by the presence of a unique outer membrane in their cell envelope. Specifically, the outer membrane consists of phospholipid in the inner leaflet and lipid A in the outer leaflet, which serves as a permeability barrier to shield Gram-negative bacteria from the damage of external detergents and antibiotics.

Potent antibiotics targeting LpxC, the second enzyme in the pathway, have been discovered; these compounds display impressive antimicrobial activity against susceptible and multidrug-resistant Gram-negative bacteria in vitro and in animal models, highlighting the therapeutic potential of disrupting lipid A biosynthesis as an effective counter measurement to combat drug-resistant Gram-negative infections. LpxC (the enzyme uridyldiphospho-3-O—(R-hydroxydecanoyl)-N-acetylglucosamine deacetylase) is present across all Gram-negative bacterial species of interest and is involved in the first committed step in outer membrane biosynthesis. Thus, LpxC is essential for survival and presents an ideal target for antibiotic activity in Gram-negative bacterial species.

Although there have been advances in the field, there remains a need for anti-bactericidal agents for Gram-negative bacteria that have an acceptable efficacy and toxicity/tolerance profile.

### SUMMARY OF THE DISCLOSURE

Aspects of the disclosure relate to LpxH targeting compounds and compositions thereof as well as methods for making and using the same. The inventors discovered that targeting downstream lipid A enzymes, such as LpxH, may offer unique advantages as inhibition of downstream lipid A enzymes not only disrupts the essential pathway of lipid A biosynthesis but also leads to the accumulation of toxic lipid A intermediates in the bacterial inner membrane, resulting in an independent mechanism of bacterial killing. LpxH is a member of the calcineurin-like phosphatases (CLPs), which catalyzes the hydrolysis of UDP-2,3-diacyl-glucosamine (UDP-DAGn) to yield lipid X and UMP. Intriguingly, such a chemical transformation is carried by three functional orthologs in distinct Gram-negative bacteria, with LpxH found in about 70% of Gram-negative bacteria and all of the World Health Organization (WHO) priority Gram-negative pathogens.

In one aspect of the disclosure, provided are LpxH targeting compounds of Formula (I) and/or salt(s) thereof:

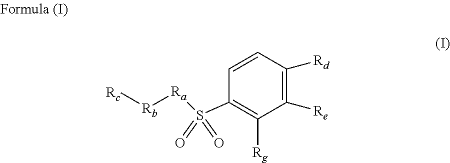

wherein
   Rₐ is selected from the group consisting of:

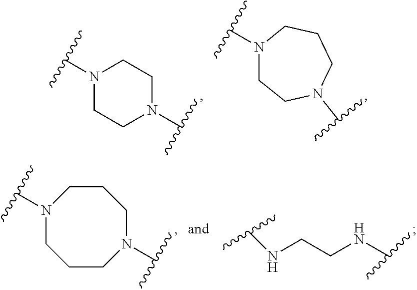 [SMILES: *N1CCN(*)CC1 ; *N1CCCN(*)CC1 ; *N1CCCN(*)CCC1 ; *NCCN*]

R_{b} is selected from the group consisting of single bond, C₄ to C₁₀ unsubstituted or substituted aryl, unsubstituted or substituted four to ten member heterocycle ring, and C₁ to C₁₀ unsubstituted or substituted alkyl;
R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, or C₁ to C₁₀ unsubstituted or substituted alkyl;
R_{d} and Rₑ are each independently hydrogen, —OH, —COH, —COH, —COC, —COOH, R_{f}, or are taken together as an unsubstituted or substituted four to eight member nitrogen containing heterocycle ring,
   wherein R_{f} is selected from the group consisting of:

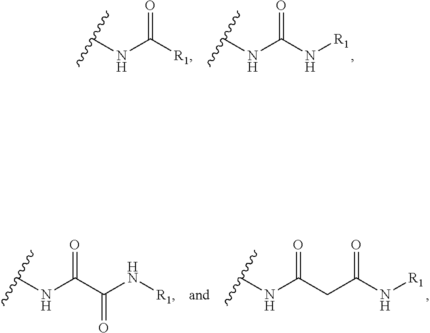

R₁ is hydrogen, methyl, —OH, C₁ to C₁₀ alkyl unsubstituted or substituted with —OH, —COH, —COH, —COC, —NHOH, —CONHOH, —COOH, an unsubstituted or substituted four to eight member heterocycle ring, or a combination thereof; and
R_{g} is hydrogen, —OH, —COH, —COH, —COC, —COOH, or C₁ to C₅ unsubstituted or substituted alkyl.

The compounds of Formula (I) and/or salts thereof may have a structure where R_{d} and Rₑ are taken together as an unsubstituted or substituted four to eight member nitrogen containing heterocycle ring. For instance, R_{d} and Rₑ may be taken together as an unsubstituted or substituted five member nitrogen containing heterocycle ring.

In some embodiments, the compounds of Formula (I) and/or salts thereof have a structure according to Formula (II):

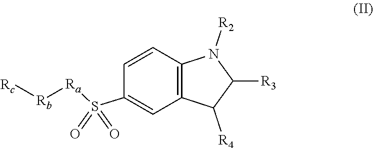

wherein,
   R₂ is a hydrogen, an aldehyde, a ketone, a C₁ to C₅ alkyl unsubstituted or substituted with —OH, —COH, —COC, —COOH, or a combination thereof, and
   R₃ and R₄ are independently hydrogen, C₁ to C₅ substituted or unsubstituted alkyl, or taken together as a double bond.

In at least one instance, R₃ and R₄ of the compounds of Formula (II) are hydrogen or taken together as a double bond. R₂ may be a hydrogen, —COH, —COC, or —COOH. R_{d} is R_{f}, Rₑ is hydrogen, —COH, —COH, —COC, or —COOH, and R_{g} is hydrogen.

Preferably, the compounds of Formula (I) and/or salts thereof have a structure where R_{d} is R_{f} and Rₑ is hydrogen. In some cases, R₁ is a C₁ to C₁₀ alkyl substituted with —OH, —COH, —COH, —COC, —NHOH, —CONHOH, and/or —COOH. In further cases, R₁ is a C₁ to C₈ alkyl substituted with —OH, —COH, —COH, —COC, —NHOH, —CONHOH, and/or —COOH. In additional cases, R₁ is a C₁ to C₆ alkyl substituted —OH, —COH, —COH, —COC, —NHOH, —CONHOH, and/or —COOH. In yet further cases, R₁ is a C₂ to C₅ alkyl substituted with —OH, —COH, —COH, —COC, —NHOH, —CONHOH, and/or —COOH.

The compounds of Formula (I) and/or salts thereof may have a structure where R₁ is selected from the group consisting of:

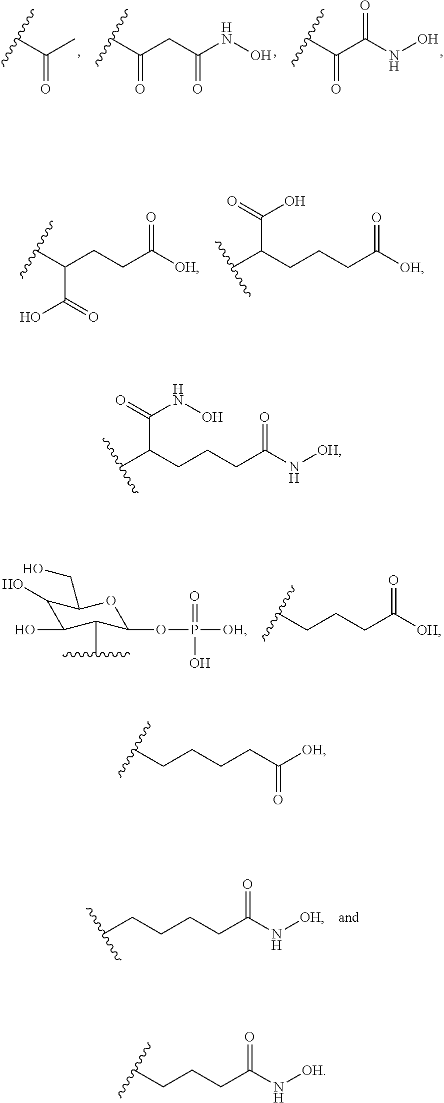

In at least one embodiment, the compound of Formula (I) has a structure in accordance with Formula (III)

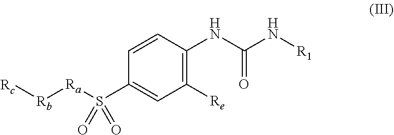

The compound of Formula (III) may have a structure where R₁ is a C₁ to C₁₀ alkyl substituted with —OH, —COH, —COH, —COC, —NHOH, —CONHOH, and/or —COOH. In some instances, R₁ of compound of Formula (III) may be selected from the group consisting of:

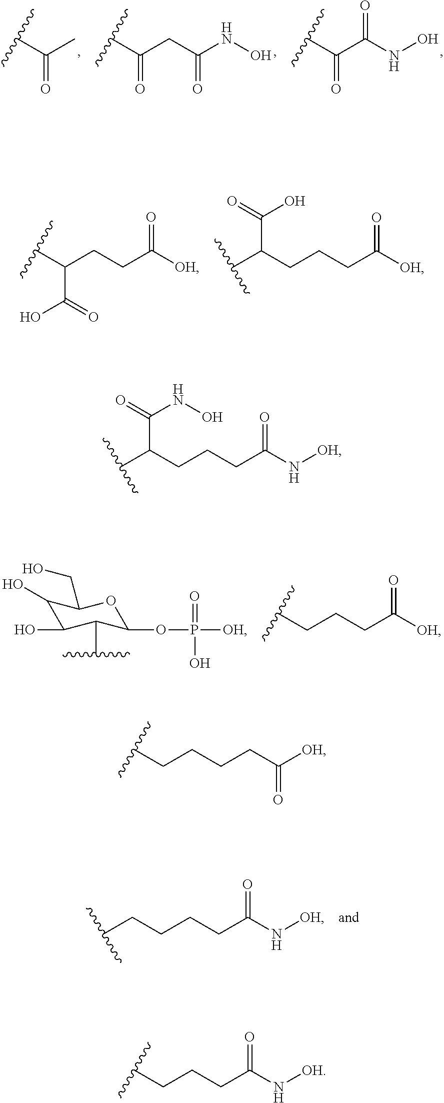

The compounds of Formula (I) and/or salts thereof may have a structure where Rₐ is

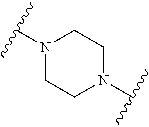 [SMILES: *N1CCN(*)CC1]

Additionally or alternatively, the compounds of Formula (I) and/or salts thereof may have a structure where R_{b} is selected from the group consisting of C₁ to C₁₀ unsubstituted or substituted alkyl; and R_{c} comprises halogen, —CO₂CH₃, —COOH, —CN₂CF₃, —CF₃, —C₂OH, —CCOH, C₄ to C₁₀ unsubstituted or substituted aryl, unsubstituted or substituted four to ten member heterocycle ring, or C₁ to C₁₀ unsubstituted or substituted alkyl. For example, R_{b} is selected from the group consisting of C₁ to C₄ unsubstituted or substituted alkyl; and R_{c} comprises halogen, —CO₂CH₃, —COOH, —CN₂CF₃, —CF₃, —C₂OH, —CCOH, C₄ to C₁₀ unsubstituted or substituted aryl, unsubstituted or substituted four to ten member heterocycle ring, or C₁ to C₁₀ unsubstituted or substituted alkyl. In some instances, R_{c} comprises hydrogen, halogen, —OH, —CO₂CH₃, —COOH, —CN₂CF₃, —CF₃, —C₂OH, —CONHOH, —CCOH, or C₁ to C₁₀ unsubstituted or substituted alkyl. In at least one embodiment, R_{c} comprises two or more of hydrogen, halogen, —OH, —CO₂CH₃, —COOH, —CN₂CF₃, —CF₃, —C₂OH, —CONHOH, —CCOH, or C₁ to C₁₀ unsubstituted or substituted alkyl.

In another aspect of the disclosure, provided is a composition comprising:
(a) an amount of a compound of Formula (I) or a salt thereof:

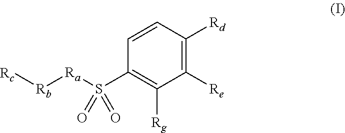

wherein
   Rₐ is selected from the group consisting of:

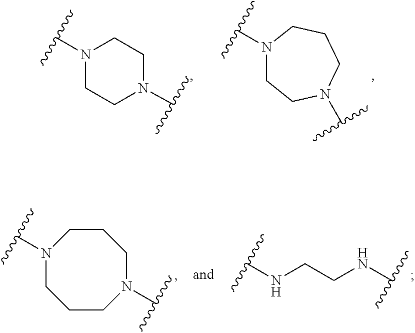 [SMILES: *N1CCN(*)CC1 ; *N1CCCN(*)CC1 ; *N1CCCN(*)CCC1 ; *NCCN*]

R_{b} is selected from the group consisting of single bond, C₄ to C₁₀ unsubstituted or substituted aryl, unsubstituted or substituted four to ten member heterocycle ring, and C₁ to C₁₀ unsubstituted or substituted alkyl;
R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, or C₁ to C₁₀ unsubstituted or substituted alkyl;
R_{d} and Rₑ are independently hydrogen, —OH, —COH, —COH, —COC, —COOH, R_{f}, or are taken together as an unsubstituted or substituted four to eight member nitrogen containing heterocycle ring,
   wherein R_{f} is selected from the group consisting of:

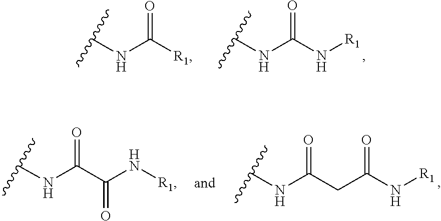

R₁ is hydrogen, methyl, —OH, C₁ to C₁₀ alkyl unsubstituted or substituted with —OH, —COH, —COH, —COC, —NHOH, —CONHOH, —COOH, an unsubstituted or substituted four to eight member heterocycle ring, or a combination thereof; and;
R_{g} is hydrogen, —OH, —COH, —COH, —COC, —COOH, or C₁ to C₅ unsubstituted or substituted alkyl; and
(b) a pharmaceutical expedient.

Preferably, the compositions disclosed herein include a therapeutically effective amount of the compound of Formula (I). For example, the therapeutically effective amount of the compound of Formula (I) may be about 1 μg or more.

According to a further aspect of the disclosure, a method is provided for treating a Gram-negative bacterial infection in a patient by administering compounds of Formula (I) and/or salts thereof or compositions thereof to a patient in need of treatment thereof.

### FIGURES OF THE DISCLOSURE

Implementation of the present technology will now be described, by way of example only, with reference to the attached figures, wherein:

FIG. 1 is a side view of the complex of KpLpxH and N-Hydroxy-5-(3-(4-((4-(3-chloro-5-(trifluoromethyl)phenyl)piperazin-1-yl)sulfonyl)phenyl)ureido)-pentanamide in accordance with aspects of the disclosure;

FIG. 2 is a top view of the complex shown in FIG. 1;

FIG. 3 is an omit (2mFo-DFc) map of N-Hydroxy-5-(3-(4-((4-(3-chloro-5-(trifluoromethyl)phenyl)piperazin-1-yl)sulfonyl)phenyl)ureido)-pentanamide bound to K. pneumoniae LpxH contoured at 0.756 according to aspects of the disclosure;

FIG. 4A is a pharmacophore model for an active LpxH inhibitor in accordance with aspects of the disclosure;

FIG. 4B is a pharmacophore model for an inactive LpxH inhibitor according to aspects of the disclosure;

FIG. 5 is an image of bacterial disk diffusion assays of Compound 1 in accordance with aspects of the disclosure;

FIG. 6A displays E. coli LpxH inhibition potency of an exemplary Compound 1 analog compound according to aspects of the disclosure;

FIG. 6B is graphs of the LC-MS/MS analysis of the E. coli LpxH/Compound 12 photo-crosslinking product in accordance with aspects of the disclosure;

FIG. 6C is an image of a mapping of F141 and L137 onto the modeled E. coli LpxH structure according to aspects of the disclosure;

FIG. 7A is an image showing the crystal structure of K. pneumoniae LpxH in complex with its product, lipid X, in accordance with aspects of the disclosure;

FIG. 7B is an image showing the crystal structure of K. pneumoniae LpxH in complex with Compound 1 according to aspects of the disclosure;

FIG. 7C is an image showing an overlay of Compound 1 with the 2-N-acyl chain of lipid X in the K. pneumoniae LpxH complex structures in accordance with aspects of the disclosure;

FIG. 8A is graph of ¹⁹F NMR measurements of two conformational states of Compound 1 bound to K. pneumoniae LpxH according to aspects of the disclosure;

FIG. 8B is a schematic of the two states of Compound 1 in the K. pneumoniae LpxH complex in accordance with aspects of the disclosure;

FIG. 8C shows a crystal structure of the K. pneumoniae LpxH/Compound 31 complex and an overlay of lipid X in the K. pneumoniae LpxH complex according to aspects of the disclosure; and

FIGS. 9A-9D are bar graphs showing LpxH inhibition and antibiotic activity of Compound 1, Compound 26, and Compound 31 in various bacteria in accordance with aspects of the disclosure.

It should be understood that the various aspects are not limited to the arrangements and instrumentality shown in the drawings.

### DETAILED DESCRIPTION OF THE DISCLOSURE

Aspects of the instant disclosure relate to LpxH targeting compounds and compositions thereof. Additional aspects relate to methods for making and using such LpxH targeting compounds and compositions thereof for treating and/or ameliorating the symptoms of patients with a Gram-negative bacteria.

The inventors recognized that Gram-negative bacteria are characterized by the presence of a unique cell wall component known as lipopolysaccharide (LPS) or lipooligosaccharide (LOS) in the bacterial outer membrane. Lipid A, a glucosamine-based phospholipid, is the hydrophobic anchor of LPS/LOS. Among the nine enzymes involved in lipid A biosynthesis, three functional orthologs (LpxH in β- and γ-proteobacteria, LpxI in α-proteobacteria, and LpxG in Chlamydiae) carry out the cleavage of the pyrophosphate group of UDP-2,3-diacylglucosamine (UDP-DAGn) to form lipid X, but they never co-exist. LpxH and LpxG are unique members of the metal-dependent calcineurin-like phosphoesterase (CLP) family. LpxH functions in the vast majority of WHO priority Gram-negative pathogens, including Pseudomonas aeruginosa, Acinetobacter baumannii, Klebsiella pneumoniae, Escherichia coli, Haemophilius influenzae, and Neisseria gonorrhoeae.

As mentioned above, the inventors discovered that targeting downstream lipid A enzymes, such as LpxH, may offer unique advantages as inhibition of downstream lipid A enzymes not only disrupts the essential pathway of lipid A biosynthesis but also leads to the accumulation of toxic lipid A intermediates in the bacterial inner membrane, resulting in an independent mechanism of bacterial killing. Without being limited to any specific theory, it is believed that certain embodiments of the compounds of Formula (I) and/or salts thereof fit into the L-shaped acyl chain-binding chamber of LpxH, e.g., with an indoline ring situated adjacent to the active site, a sulfonyl group adopting a sharp kink, and a N—CF₃-phenyl substituted piperazine group reaching to the far side of the LpxH acyl chain-binding chamber.

In one aspect of the disclosure, provided are compounds of Formula (I) and/or salts thereof:

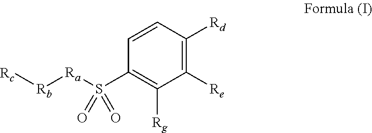

wherein
   Rₐ is selected from the group consisting of:

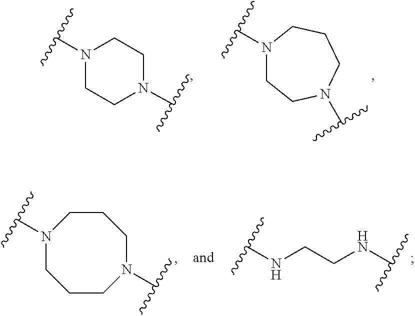 [SMILES: *N1CCN(*)CC1 ; *N1CCCN(*)CC1 ; *N1CCCN(*)CCC1 ; *NCCN*]

R_{b} is selected from the group consisting of single bond, C₄ to C₁₀ unsubstituted or substituted aryl, unsubstituted or substituted four to ten member heterocycle ring, and C₁ to C₁₀ unsubstituted or substituted alkyl;
R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, or C₁ to C₁₀ unsubstituted or substituted alkyl;
R_{d} and Rₑ are independently hydrogen, —OH, —COH, —COH, —COC, —COOH, R_{f}, or are taken together as an unsubstituted or substituted four to eight member nitrogen containing heterocycle ring,
   wherein R_{f} is selected from the group consisting of:

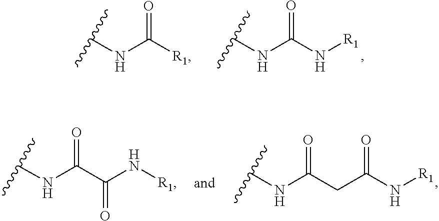

R₁ is hydrogen, methyl, —OH, C₁ to C₁₀ alkyl unsubstituted or substituted with —OH, —COH, —COH, —COC, —NHOH, —CONHOH, —COOH, an unsubstituted or substituted four to eight member heterocycle ring, or a combination thereof; and
R_{g} is hydrogen, —OH, —COH, —COH, —COC, —COOH, or C₁ to C₅ unsubstituted or substituted alkyl.

The compounds of Formula (I) and/or salts thereof preferably have a structure where Rₐ is:

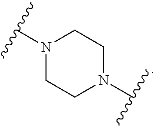 [SMILES: *N1CCN(*)CC1]

R_{b} of the compounds of Formula (I) and/or salts thereof is typically selected from the group consisting of single bond, C₄ to C₁₀ unsubstituted or substituted aryl, unsubstituted or substituted four to ten member heterocycle ring, and C₁ to C₁₀ unsubstituted or substituted alkyl. R_{b} may be chosen from C₄ to C₁₀ unsubstituted or substituted aryls, such as C₄ to C₉ aryls, C₄ to C₈ aryls, C₄ to C₇ aryls, C₄ to C₆ aryls, or C₅ to C₆ aryls. In at least one embodiment, R_{b} is a C₆ unsubstituted or substituted aryl.

The compounds of Formula (I) and/or salts thereof may have a structure where R_{b} is an unsubstituted or substituted four to ten member heterocycle ring. For example, R_{b} may be an unsubstituted or substituted four member heterocycle ring, five member heterocycle ring, six member heterocycle ring, seven member heterocycle ring, eight member heterocycle ring, nine member heterocycle ring, ten member heterocycle ring, or any range formed therefrom. The heterocycles may contain at least one nitrogen silicon, oxygen, phosphorous, and/or sulfur. In some cases, the four to ten member heterocycle ring of R_{b} may contain two or more nitrogen silicon, oxygen, phosphorous, sulfur, or a combination thereof.

R_{b} may be a C₁ to C₁₀ unsubstituted or substituted alkyl, such as those chosen from C₁ to C₉ alkyls, C₁ to C₈ alkyls, C₁ to C₇ alkyls, C₁ to C₆ alkyls, C₁ to C₅ alkyls, C₁ to C₄ alkyls, C₁ to C₃ alkyls, C₁ to C₂ alkyls; C₂ to C₁₀ alkyls, C₂ to C₉ alkyls, C₂ to C₈ alkyls, C₂ to C₇ alkyls, C₂ to C₆ alkyls, C₂ to C₅ alkyls, C₂ to C₄ alkyls, C₂ to C₃ alkyls; C₃ to C₁₀ alkyls, C₃ to C₉ alkyls, C₃ to C₈ alkyls, C₃ to C₇ alkyls, C₃ to C₆ alkyls, C₃ to C₅ alkyls, C₃ to C₄ alkyls; C₄ to C₁₀ alkyls, C₄ to C₉ alkyls, C₄ to C₈ alkyls, C₄ to C₇ alkyls, C₄ to C₆ alkyls, C₄ to C₅ alkyls; C₅ to C₁₀ alkyls, C₅ to C₉ alkyls, C₅ to C₈ alkyls, C₅ to C₇ alkyls, C₅ to C₆ alkyls; C₆ to C₁₀ alkyls, C₆ to C₉ alkyls, C₆ to C₈ alkyls, C₆ to C₇ alkyls; C₇ to C₁₀ alkyls, C₇ to C₉ alkyls, C₇ to C₈ alkyls; C₈ to C₁₀alkyls, and C₈ to C₉ alkyls. For example, R_{b} may be a methyl, ethyl, n-propyl, i-propyl, n-butyl, or t-butyl.

The compounds of Formula (I) and/or salts thereof typically have a structure where R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, or C₁ to C₁₀ unsubstituted or substituted alkyl. In some cases, R_{c} comprises two or more hydrogen, halogen, —OH, —CO₂CH₃, —COOH, —CN₂CF₃, —CF₃, —C₂OH, —CONHOH, —CCOH, or C₁ to C₁₀ unsubstituted or substituted alkyl. For example, two or more of the groups of R_{c} may be coupled to the same or different atoms (e.g., the same or different carbon atoms) of R_{b}. In at least one instance, the two or more groups of R_{c} may be —CF₃, chlorines, bromines, or flourines, which are believed to provide surprising improvement in antibiotic activity against wild-type K. pneumoniae.

R_{c} may be a halogen, such as fluorine (F), chlorine (Cl), bromine (Br), iodine (I), and astatine (At). In some embodiment, R_{c} is —OH, —CO₂CH₃, —COOH, —CN₂CF₃, —CF₃, —C₂OH, —CONHOH, or —CCOH. In further embodiments, R_{c} is a C₄ to C₁₀ unsubstituted or substituted aryl, such as those chosen from C₄ to C₉ aryls, C₄ to C₈ aryls, C₄ to C₇ aryls, C₄ to C₆ aryls, and C₅ to C₆ aryls. In at least one embodiment, R_{c} is a C₆ unsubstituted or substituted aryl. R_{c} may be a unsubstituted or substituted four to ten member heterocycle ring. For example, R_{c} may be an unsubstituted or substituted four member heterocycle ring, five member heterocycle ring, six member heterocycle ring, seven member heterocycle ring, eight member heterocycle ring, nine member heterocycle ring, ten member heterocycle ring, or any range formed therefrom. The heterocycles may contain at least one nitrogen silicon, oxygen, phosphorous, and/or sulfur. In some cases, the four to ten member heterocycle ring of R_{c} may contain two or more nitrogen silicon, oxygen, phosphorous, sulfur, or a combination thereof.

R_{c} may be a C₁ to C₁₀ unsubstituted or substituted alkyl, such as those chosen from C₁ to C₉ alkyls, C₁ to C₈ alkyls, C₁ to C₇ alkyls, C₁ to C₆ alkyls, C₁ to C₅ alkyls, C₁ to C₄ alkyls, C₁ to C₃ alkyls, C₁ to C₂ alkyls; C₂ to C₁₀ alkyls, C₂ to C₉ alkyls, C₂ to C₈ alkyls, C₂ to C₇ alkyls, C₂ to C₆ alkyls, C₂ to C₅ alkyls, C₂ to C₄ alkyls, C₂ to C₃ alkyls; C₃ to C₁₀ alkyls, C₃ to C₉ alkyls, C₃ to C₈ alkyls, C₃ to C₇ alkyls, C₃ to C₆ alkyls, C₃ to C₅ alkyls, C₃ to C₄ alkyls; C₄ to C₁₀ alkyls, C₄ to C₉ alkyls, C₄ to C alkyls, C₄ to C₇ alkyls, C₄ to C₆ alkyls, C₄ to C₅ alkyls; C₅ to C₁₀ alkyls, C₅ to C₉ alkyls, C₅ to C₈ alkyls, C₅ to C₇ alkyls, C₅ to C₆ alkyls; C₆ to C₁₀ alkyls, C₆ to C₉ alkyls, C₆ to CG alkyls, C₆ to C₇ alkyls; C₇ to C₁₀ alkyls, C₇ to C₉ alkyls, C₇ to C₈ alkyls; C₈ to C₁₀alkyls, and C₈ to C₉ alkyls. For example, R_{c} may be a methyl, ethyl, n-propyl, i-propyl, n-butyl, or t-butyl.

In some embodiments, the compounds of Formula (I) and/or salts thereof have a structure where R_{b} is selected from the group consisting of hydrogen, C₁ to C₁₀ unsubstituted or substituted alkyl, while R_{c} comprises halogen, —CO₂CH₃, —COOH, —CN₂CF₃, —CF₃, —C₂OH, —CCOH, C₄ to C₁₀ unsubstituted or substituted aryl, unsubstituted or substituted four to ten member heterocycle ring, or C₁ to C₁₀ unsubstituted or substituted alkyl. For instance, R_{b} may be selected from the group consisting of hydrogen, C₁ to C₄ unsubstituted or substituted alkyl, while R_{c} comprises halogen, —CO₂CH₃, —COOH, —CN₂CF₃, —CF₃, —C₂OH, —CCOH, C₄ to C₁₀ unsubstituted or substituted aryl, unsubstituted or substituted four to ten member heterocycle ring, or C₁ to C₁₀ unsubstituted or substituted alkyl. In some embodiment, R_{b} is a single bond and R_{c} is a C₄ to C₁₀ aryl substituted with halogen, —OH, —CO₂CH₃, —COOH, —CN₂CF₃, —CF₃, —C₂OH, —CCOH. Preferably, the substituent of R_{c} is in the m-position, when applicable based on the structure of R_{c}.

In other embodiments, the compounds of Formula (I) and/or salts thereof have a structure where R_{b} is selected from the group consisting of C₄ to C₁₀ unsubstituted or substituted aryl, and unsubstituted or substituted four to ten member heterocycle ring, while R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, C₁ to C₁₀ unsubstituted alkyl, or C₁ to C₁₀ substituted alkyl. In one embodiment, R_{b} is a C₄ to C₁₀ unsubstituted or substituted aryl, such as a C₆ unsubstituted aryl, and R_{c} comprises one or more of halogen, —CN₂CF₃, or —CF₃. In at least one preferred embodiment, R_{c} comprises a halogen, —CN₂CF₃, or —CF₃ in the m-position of R_{b}, e.g., when R_{b} is a C₆ aryl. For instance, R_{b} may be a C₆ aryl with R_{c} being a fluorine, chlorine, bromine, or —CF₃ in the m-position of R_{b}.

In at least one embodiment, the compounds of Formula (I) and/or salts thereof may have a structure in accordance with Formula (IV):

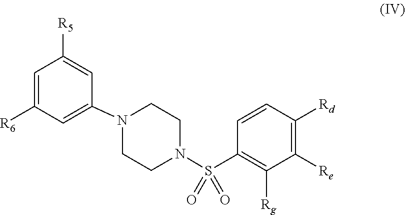

R₅ and R₆ may independently be hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, C₁ to C₁₀ unsubstituted or substituted alkyl, or a combination thereof. For instance, R₅ and R₆ may independently be chosen from a fluorine, chlorine, bromine, —CF₃, and a combination thereof. R₅ and R₆ may independently be chosen from the C₄ to C₁₀ unsubstituted aryl, the C₄ to C₁₀ substituted aryl, the unsubstituted or substituted four to ten member heterocycle ring, the C₁ to C₁₀ unsubstituted alkyl, and the C₁ to C₁₀ substituted alkyl described herein with reference to R_{c}.

The compounds of Formula (I) and/or salts thereof typically have a structure where R_{d} and Rₑ are independently hydrogen, —OH, —COH, —COH, —COC, —COOH, R_{f}, or are taken together as an unsubstituted or substituted four to eight member nitrogen containing heterocycle ring. In one embodiment, Rₑ is a hydrogen, —OH, —COH, —COH, —COC, or —COOH. For instance, R_{d} is R_{f}, while Rₑ may be a hydrogen, —OH, —COH, —COH, —COC, or —COOH. Preferably, R_{d} is R_{f} and Rₑ is a hydrogen. R_{f} is typically selected from the group consisting of:

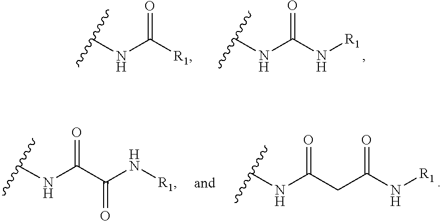

In some embodiments, the compound of Formula (I) has a structure in accordance with Formula (III):

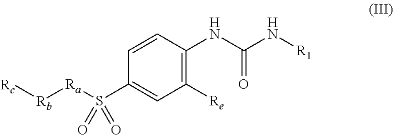

R₁ is typically hydrogen, methyl, —OH, C₁ to C₁₀ unsubstituted alkyl, C₁ to C₁₀ alkyl substituted with —OH, —COH, —COH, —COC, —NHOH, —CONHOH, —COOH, an unsubstituted or substituted four to eight member heterocycle ring, or a combination thereof. For example, R₁ may be a C₁ to C₁₀ unsubstituted or substituted alkyl, such as those chosen from C₁ to C₉ alkyls, C₁ to C₈ alkyls, C₁ to C₇ alkyls, C₁ to C₆ alkyls, C₁ to C₅ alkyls, C₁ to C₄ alkyls, C₁ to C₃ alkyls, C₁ to C₂ alkyls; C₂ to C₁₀ alkyls, C₂ to C₉ alkyls, C₂ to C₈ alkyls, C₂ to C₇ alkyls, C₂ to C₆ alkyls, C₂ to C₅ alkyls, C₂ to C₄ alkyls, C₂ to C₃ alkyls; C₃ to C₁₀ alkyls, C₃ to C₉ alkyls, C₃ to C₈ alkyls, C₃ to C₇ alkyls, C₃ to C₆ alkyls, C₃ to C₅ alkyls, C₃ to C₄ alkyls; C₄ to C₁₀ alkyls, C₄ to C₉ alkyls, C₄ to C₈ alkyls, C₄ to C₇ alkyls, C₄ to C₆ alkyls, C₄ to C₅ alkyls; C₅ to C₁₀ alkyls, C₅ to C₉ alkyls, C₅ to C₈ alkyls, C₅ to C₇ alkyls, C₅ to C₆ alkyls; C₆ to C₁₀ alkyls, C₆ to C₉ alkyls, C₆ to C₈ alkyls, C₆ to C₇ alkyls; C₇ to C₁₀ alkyls, C₇ to C₉ alkyls, C₇ to C₈ alkyls; C₈ to C₁₀ alkyls, or C₈ to C₉ alkyls. For example, R₁ may be a methyl, ethyl, n-propyl, i-propyl, n-butyl, or t-butyl. In some cases, R₁ may be a C₁ to C₁₀ alkyl substituted with —OH, —COH, —COH, —COC, —NHOH, —CONHOH, —COOH, or an unsubstituted or substituted four to eight member heterocycle ring. For example, the C₁ to C₁₀ alkyl may be substituted with a four member containing heterocycle ring, five member containing heterocycle ring, six member containing heterocycle ring, seven member containing heterocycle ring, or eight member containing heterocycle ring. The four to eight member heterocycle ring may contain, e.g., at least one nitrogen silicon, oxygen, phosphorous, and/or sulfur. The four to eight member heterocycle ring substituent may be further substituted with one or more substituents as discussed below.

In some embodiments, R₁ is selected from the group consisting of:

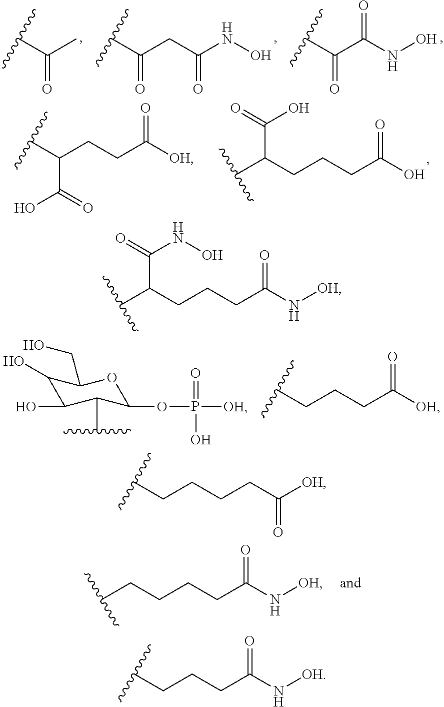

Alternatively, R_{d} and Rₑ may be taken together as an unsubstituted or substituted four to eight member nitrogen containing heterocycle ring. For example, the R_{d} and Rₑ may be taken together as a four member nitrogen containing heterocycle ring, a five member nitrogen containing heterocycle ring, a six member nitrogen containing heterocycle ring, a seven member nitrogen containing heterocycle ring, a eight member nitrogen containing heterocycle ring.

In some embodiments, R_{d} and Rₑ are taken together as an unsubstituted or substituted five member nitrogen containing heterocycle ring. For instance, R_{d} and Rₑ may be taken together as an unsubstituted or substituted five member nitrogen containing heterocycle ring, such that the compound of Formula (I) has a structure according to Formula (II):

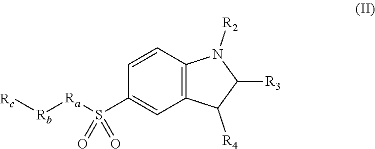

R₂ is typically a hydrogen, an aldehyde, a ketone, an unsubstituted C₁ to C₅ alkyl, a C₁ to C₅ alkyl substituted with an aldehyde group, ketone group, or carboxylic acid group. In some cases, R₂ is a C₁ to C₅ unsubstituted or substituted alkyl, such as those chosen from C₁ to C₅ alkyls, C₁ to C₄ alkyls, C₁ to C₃ alkyls, C₁ to C₂ alkyls, C₂ to C₅ alkyls, C₂ to C₄ alkyls, C₂ to C₃ alkyls, C₃ to C₅ alkyls, C₃ to C₄ alkyls, and C₄ to C₅ alkyls. For example, R₂ may be a methyl, ethyl, n-propyl, i-propyl, n-butyl, or t-butyl. The C₁ to C₅ alkyl may be substituted with alcohol group (—OH), aldehyde group (—COH), ketone group (—COC), carboxylic acid group (—COOH), or a combination thereof. Preferably, R₂ is a hydrogen, —COH, —COC, or —COOH.

R₃ and R₄ may independently be hydrogen, C₁ to C₅ substituted or unsubstituted alkyl, or taken together as a double bond. R₃ and R₄ may independently be a C₁ to C₅ unsubstituted or substituted alkyl, such as those chosen from C₁ to C₅ alkyls, C₁ to C₄ alkyls, C₁ to C₃ alkyls, C₁ to C₂ alkyls, C₂ to C₅ alkyls, C₂ to C₄ alkyls, C₂ to C₃ alkyls, C₃ to C₅ alkyls, C₃ to C₄ alkyls, or C₄ to C₅ alkyls. For example, R₃ and R₄ may independently be a methyl, ethyl, n-propyl, i-propyl, n-butyl, or t-butyl. The C₁ to C₅ alkyl may be substituted with alcohol group (—OH), aldehyde group (—COH), ketone group (—COC), carboxylic acid group (—COOH), or a combination thereof. In some embodiments, R₃ and R₄ may both be hydrogen or taken together as a double bond.

The compounds of Formula (I) and/or salts thereof typically have a structure where R_{g} is hydrogen, —OH, —COH, —COH, —COC, —COOH, C₁ to C₅ unsubstituted alkyl, or C₁ to C₅ substituted alkyl. For example, R_{g} may be a C₁ to C₅ unsubstituted or substituted alkyl, such as those chosen from C₁ to C₅ alkyls, C₁ to C₄ alkyls, C₁ to C₃ alkyls, C₁ to C₂ alkyls, C₂ to C₅ alkyls, C₂ to C₄ alkyls, C₂ to C₃ alkyls, C₃ to C₅ alkyls, C₃ to C₄ alkyls, and C₄ to C₅ alkyls. For example, R_{g} may be a methyl, ethyl, n-propyl, i-propyl, n-butyl, or t-butyl.

The compounds of Formula (I) and/or salts thereof may have a structure in accordance with one of:

| Compound | |
|---|---|
| No. | Structure |
| 1 | 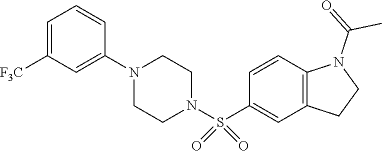 [SMILES: CC(=O)N1CCc2cc(S(=O)(=O)N3CCN(c4cccc(C(F)(F)F)c4)CC3)ccc21] |
| 2 | 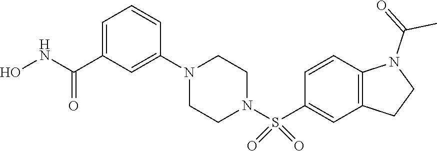 [SMILES: CC(=O)N1CCc2cc(S(=O)(=O)N3CCN(c4cccc(C(=O)NO)c4)CC3)ccc21] |
| 3 | 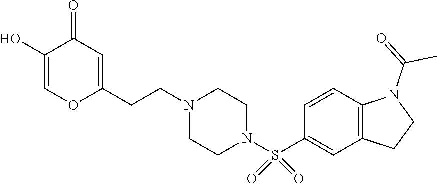 [SMILES: CC(=O)N1CCc2cc(S(=O)(=O)N3CCN(CCc4cc(=O)c(O)co4)CC3)ccc21] |
| 4 | 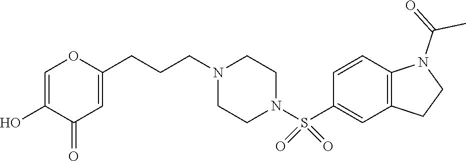 [SMILES: CC(=O)N1CCc2cc(S(=O)(=O)N3CCN(CCCc4cc(=O)c(O)co4)CC3)ccc21] |
| 5 | 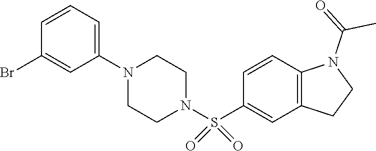 [SMILES: CC(=O)N1CCc2cc(S(=O)(=O)N3CCN(c4cccc(Br)c4)CC3)ccc21] |
| 6 | 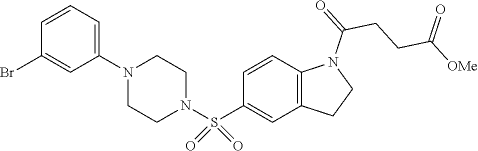 [SMILES: COC(=O)CCC(=O)N1CCc2cc(S(=O)(=O)N3CCN(c4cccc(Br)c4)CC3)ccc21] |
| 7 | 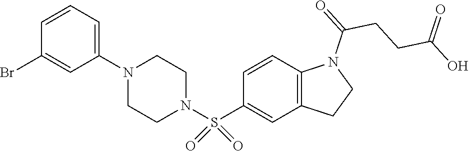 [SMILES: O=C(O)CCC(=O)N1CCc2cc(S(=O)(=O)N3CCN(c4cccc(Br)c4)CC3)ccc21] |
| 8 | 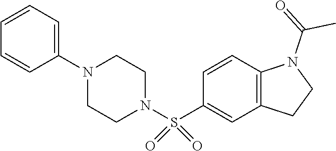 [SMILES: CC(=O)N1CCc2cc(S(=O)(=O)N3CCN(c4ccccc4)CC3)ccc21] |
| 9 | 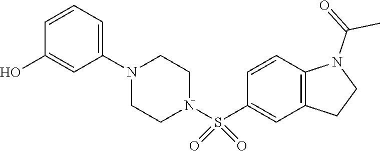 [SMILES: CC(=O)N1CCc2cc(S(=O)(=O)N3CCN(c4cccc(O)c4)CC3)ccc21] |
| 10 | 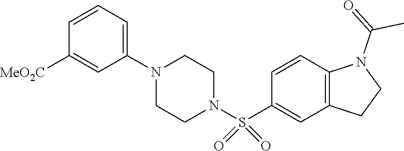 [SMILES: COC(=O)c1cccc(N2CCN(S(=O)(=O)c3ccc4c(c3)CCN4C(C)=O)CC2)c1] |
| 11 | 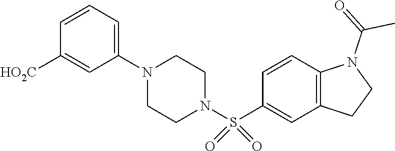 [SMILES: CC(=O)N1CCc2cc(S(=O)(=O)N3CCN(c4cccc(C(=O)O)c4)CC3)ccc21] |
| 12 | 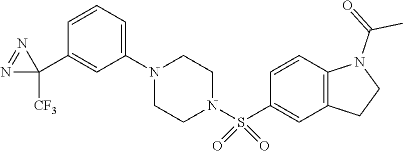 [SMILES: CC(=O)N1CCc2cc(S(=O)(=O)N3CCN(c4cccc(C5(C(F)(F)F)N=N5)c4)CC3)ccc21] |
| 13 | 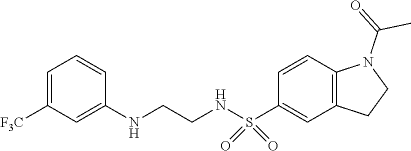 [SMILES: CC(=O)N1CCc2cc(S(=O)(=O)NCCNc3cccc(C(F)(F)F)c3)ccc21] |
| 14 | 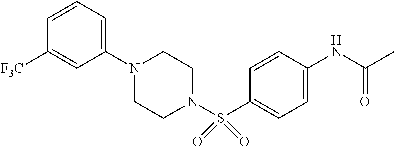 [SMILES: CC(=O)Nc1ccc(S(=O)(=O)N2CCN(c3cccc(C(F)(F)F)c3)CC2)cc1] |
| 15 | 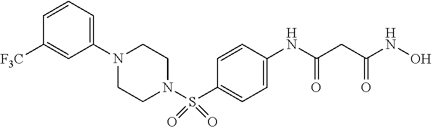 [SMILES: O=C(CC(=O)Nc1ccc(S(=O)(=O)N2CCN(c3cccc(C(F)(F)F)c3)CC2)cc1)NO] |
| 16 | 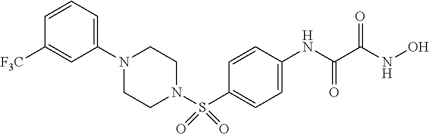 [SMILES: O=C(NO)C(=O)Nc1ccc(S(=O)(=O)N2CCN(c3cccc(C(F)(F)F)c3)CC2)cc1] |
| 17 | 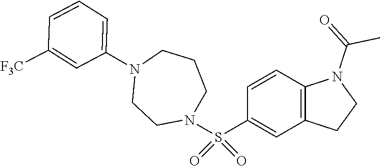 [SMILES: CC(=O)N1CCc2cc(S(=O)(=O)N3CCCN(c4cccc(C(F)(F)F)c4)CC3)ccc21] |
| 18 | 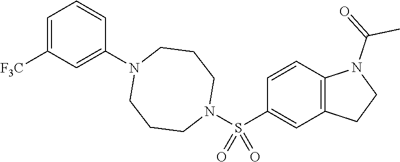 [SMILES: CC(=O)N1CCc2cc(S(=O)(=O)N3CCCN(c4cccc(C(F)(F)F)c4)CCC3)ccc21] |
| 19 | 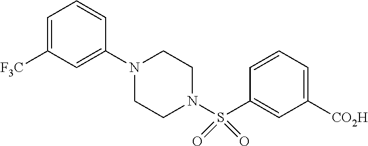 [SMILES: O=C(O)c1cccc(S(=O)(=O)N2CCN(c3cccc(C(F)(F)F)c3)CC2)c1] |
| 20 | 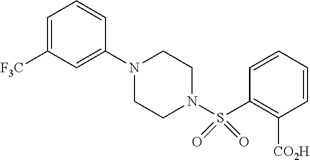 [SMILES: O=C(O)c1ccccc1S(=O)(=O)N1CCN(c2cccc(C(F)(F)F)c2)CC1] |
| 21 | 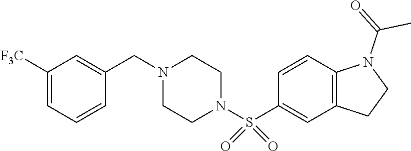 [SMILES: CC(=O)N1CCc2cc(S(=O)(=O)N3CCN(Cc4cccc(C(F)(F)F)c4)CC3)ccc21] |
| 22 | 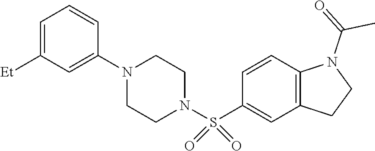 [SMILES: CCc1cccc(N2CCN(S(=O)(=O)c3ccc4c(c3)CCN4C(C)=O)CC2)c1] |
| 23 | 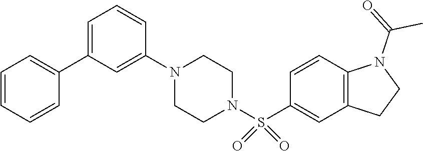 [SMILES: CC(=O)N1CCc2cc(S(=O)(=O)N3CCN(c4cccc(-c5ccccc5)c4)CC3)ccc21] |
| 24 | 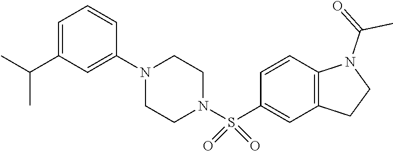 [SMILES: CC(=O)N1CCc2cc(S(=O)(=O)N3CCN(c4cccc(C(C)C)c4)CC3)ccc21] |
| 25 | 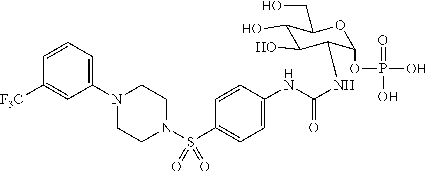 [SMILES: O=C(Nc1ccc(S(=O)(=O)N2CCN(c3cccc(C(F)(F)F)c3)CC2)cc1)NC1C(OP(=O)(O)O)OC(CO)C(O)C1O] |
| 26 | 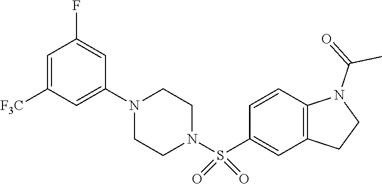 [SMILES: CC(=O)N1CCc2cc(S(=O)(=O)N3CCN(c4cc(F)cc(C(F)(F)F)c4)CC3)ccc21] |
| 27 | 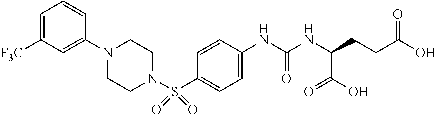 [SMILES: O=C(O)CC[C@H](NC(=O)Nc1ccc(S(=O)(=O)N2CCN(c3cccc(C(F)(F)F)c3)CC2)cc1)C(=O)O] |
| 28 | 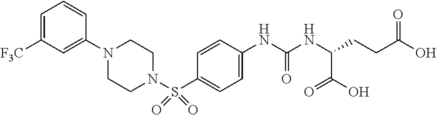 [SMILES: O=C(O)CC[C@@H](NC(=O)Nc1ccc(S(=O)(=O)N2CCN(c3cccc(C(F)(F)F)c3)CC2)cc1)C(=O)O] |
| 29 | 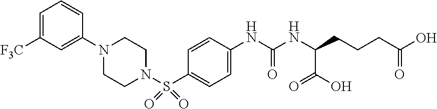 [SMILES: O=C(O)CCC[C@H](NC(=O)Nc1ccc(S(=O)(=O)N2CCN(c3cccc(C(F)(F)F)c3)CC2)cc1)C(=O)O] |
| 30 | 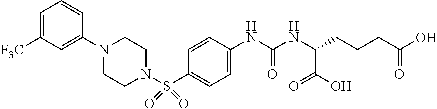 [SMILES: O=C(O)CCC[C@@H](NC(=O)Nc1ccc(S(=O)(=O)N2CCN(c3cccc(C(F)(F)F)c3)CC2)cc1)C(=O)O] |
| 31 | 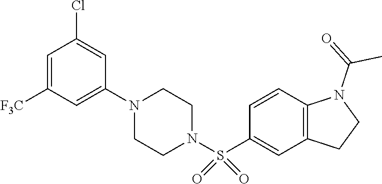 [SMILES: CC(=O)N1CCc2cc(S(=O)(=O)N3CCN(c4cc(Cl)cc(C(F)(F)F)c4)CC3)ccc21] |
| 32 | 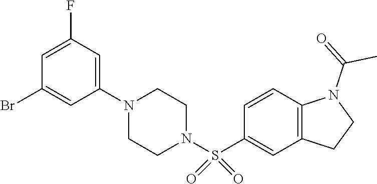 [SMILES: CC(=O)N1CCc2cc(S(=O)(=O)N3CCN(c4cc(F)cc(Br)c4)CC3)ccc21] |
| 33 | 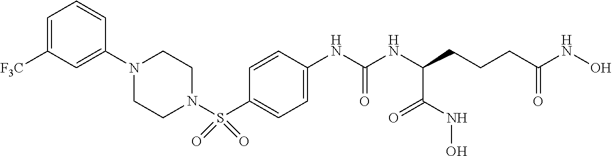 [SMILES: O=C(CCC[C@H](NC(=O)Nc1ccc(S(=O)(=O)N2CCN(c3cccc(C(F)(F)F)c3)CC2)cc1)C(=O)NO)NO] |
| 34 | 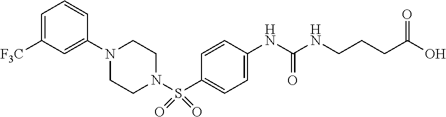 [SMILES: O=C(O)CCCNC(=O)Nc1ccc(S(=O)(=O)N2CCN(c3cccc(C(F)(F)F)c3)CC2)cc1] |
| 35 | 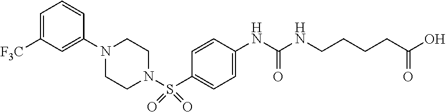 [SMILES: O=C(O)CCCCNC(=O)Nc1ccc(S(=O)(=O)N2CCN(c3cccc(C(F)(F)F)c3)CC2)cc1] |
| 36 | 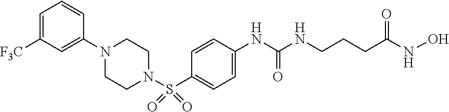 [SMILES: O=C(CCCNC(=O)Nc1ccc(S(=O)(=O)N2CCN(c3cccc(C(F)(F)F)c3)CC2)cc1)NO] |
| 37 | 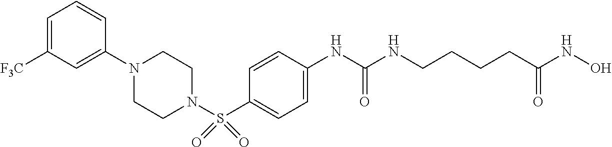 [SMILES: O=C(CCCCNC(=O)Nc1ccc(S(=O)(=O)N2CCN(c3cccc(C(F)(F)F)c3)CC2)cc1)NO] |
| 38 | 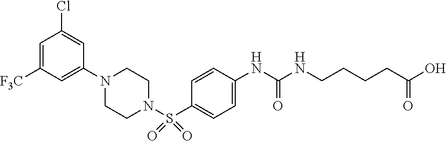 [SMILES: O=C(O)CCCCNC(=O)Nc1ccc(S(=O)(=O)N2CCN(c3cc(Cl)cc(C(F)(F)F)c3)CC2)cc1] |
| 39 | 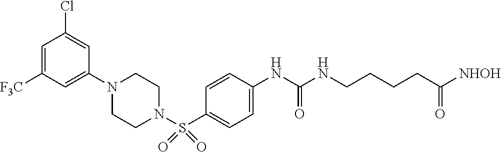 |
| 40 | 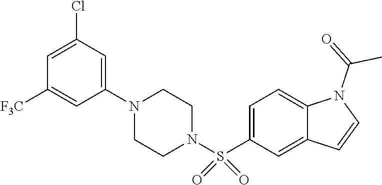 [SMILES: CC(=O)n1ccc2cc(S(=O)(=O)N3CCN(c4cc(Cl)cc(C(F)(F)F)c4)CC3)ccc21] |
| 41 | 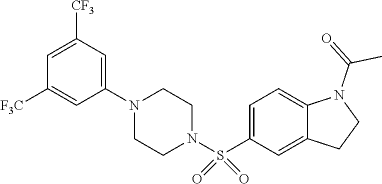 [SMILES: CC(=O)N1CCc2cc(S(=O)(=O)N3CCN(c4cc(C(F)(F)F)cc(C(F)(F)F)c4)CC3)ccc21] |
| 42 | 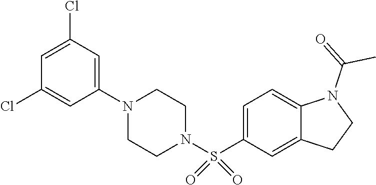 [SMILES: CC(=O)N1CCc2cc(S(=O)(=O)N3CCN(c4cc(Cl)cc(Cl)c4)CC3)ccc21] |

In one embodiment, the compounds of Formula (I) and/or salts thereof may have a structure in accordance with one of:

| Compound | |
|---|---|
| No. | Structure |
| 1 | 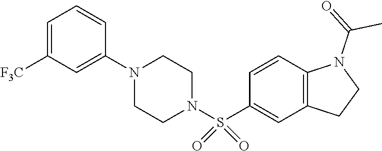 [SMILES: CC(=O)N1CCc2cc(S(=O)(=O)N3CCN(c4cccc(C(F)(F)F)c4)CC3)ccc21] |
| 2 | 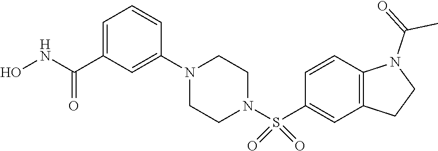 [SMILES: CC(=O)N1CCc2cc(S(=O)(=O)N3CCN(c4cccc(C(=O)NO)c4)CC3)ccc21] |
| 3 | 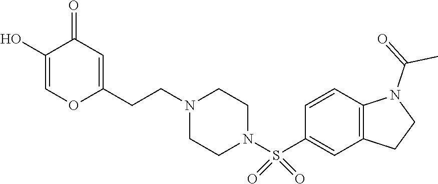 [SMILES: CC(=O)N1CCc2cc(S(=O)(=O)N3CCN(CCc4cc(=O)c(O)co4)CC3)ccc21] |
| 4 | 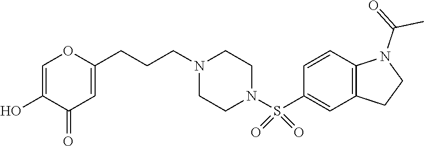 [SMILES: CC(=O)N1CCc2cc(S(=O)(=O)N3CCN(CCCc4cc(=O)c(O)co4)CC3)ccc21] |
| 5 | 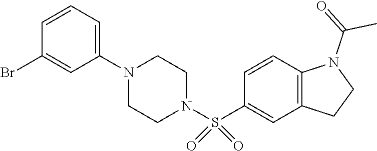 [SMILES: CC(=O)N1CCc2cc(S(=O)(=O)N3CCN(c4cccc(Br)c4)CC3)ccc21] |
| 6 | 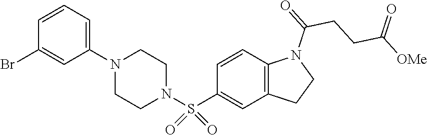 [SMILES: COC(=O)CCC(=O)N1CCc2cc(S(=O)(=O)N3CCN(c4cccc(Br)c4)CC3)ccc21] |
| 7 | 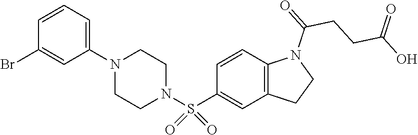 [SMILES: O=C(O)CCC(=O)N1CCc2cc(S(=O)(=O)N3CCN(c4cccc(Br)c4)CC3)ccc21] |
| 8 | 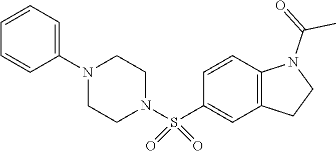 [SMILES: CC(=O)N1CCc2cc(S(=O)(=O)N3CCN(c4ccccc4)CC3)ccc21] |
| 9 | 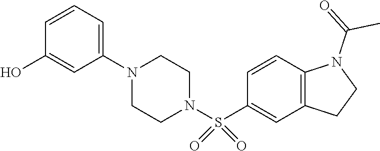 [SMILES: CC(=O)N1CCc2cc(S(=O)(=O)N3CCN(c4cccc(O)c4)CC3)ccc21] |
| 10 | 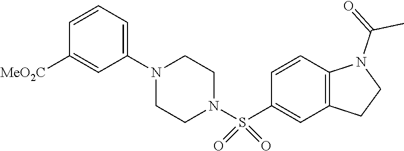 [SMILES: COC(=O)c1cccc(N2CCN(S(=O)(=O)c3ccc4c(c3)CCN4C(C)=O)CC2)c1] |
| 11 | 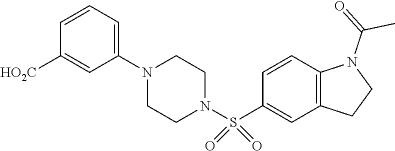 [SMILES: CC(=O)N1CCc2cc(S(=O)(=O)N3CCN(c4cccc(C(=O)O)c4)CC3)ccc21] |
| 12 | 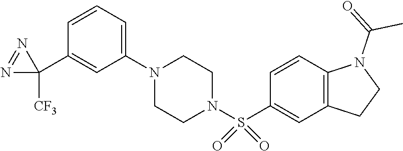 [SMILES: CC(=O)N1CCc2cc(S(=O)(=O)N3CCN(c4cccc(C5(C(F)(F)F)N=N5)c4)CC3)ccc21] |
| 13 | 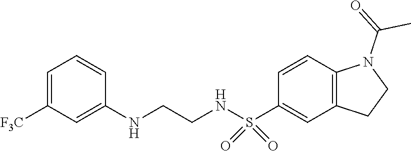 [SMILES: CC(=O)N1CCc2cc(S(=O)(=O)NCCNc3cccc(C(F)(F)F)c3)ccc21] |
| 17 | 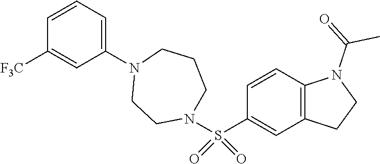 [SMILES: CC(=O)N1CCc2cc(S(=O)(=O)N3CCCN(c4cccc(C(F)(F)F)c4)CC3)ccc21] |
| 18 | 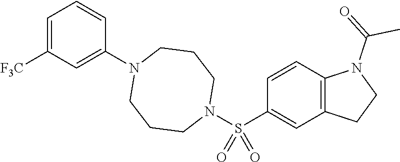 [SMILES: CC(=O)N1CCc2cc(S(=O)(=O)N3CCCN(c4cccc(C(F)(F)F)c4)CCC3)ccc21] |
| 21 | 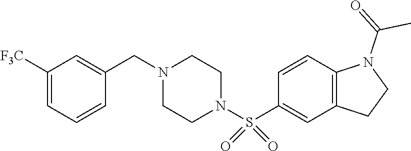 [SMILES: CC(=O)N1CCc2cc(S(=O)(=O)N3CCN(Cc4cccc(C(F)(F)F)c4)CC3)ccc21] |
| 22 | 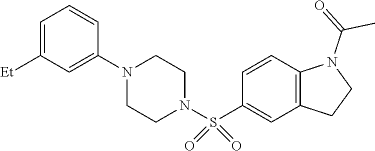 [SMILES: CCc1cccc(N2CCN(S(=O)(=O)c3ccc4c(c3)CCN4C(C)=O)CC2)c1] |
| 23 | 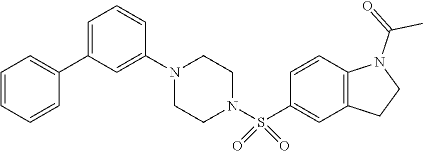 [SMILES: CC(=O)N1CCc2cc(S(=O)(=O)N3CCN(c4cccc(-c5ccccc5)c4)CC3)ccc21] |
| 24 | 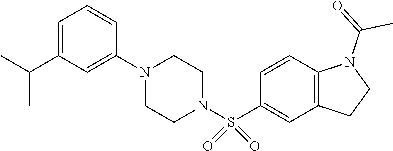 [SMILES: CC(=O)N1CCc2cc(S(=O)(=O)N3CCN(c4cccc(C(C)C)c4)CC3)ccc21] |
| 26 | 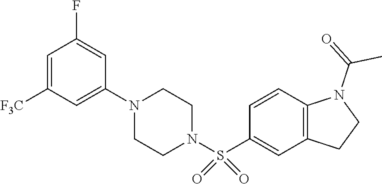 [SMILES: CC(=O)N1CCc2cc(S(=O)(=O)N3CCN(c4cc(F)cc(C(F)(F)F)c4)CC3)ccc21] |
| 31 | 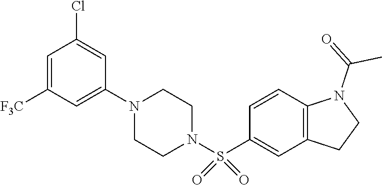 [SMILES: CC(=O)N1CCc2cc(S(=O)(=O)N3CCN(c4cc(Cl)cc(C(F)(F)F)c4)CC3)ccc21] |
| 32 | 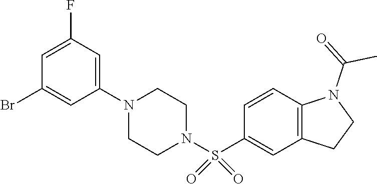 [SMILES: CC(=O)N1CCc2cc(S(=O)(=O)N3CCN(c4cc(F)cc(Br)c4)CC3)ccc21] |
| 40 | 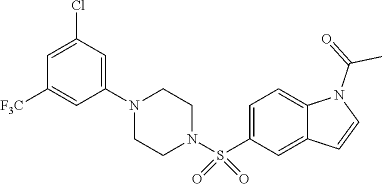 [SMILES: CC(=O)n1ccc2cc(S(=O)(=O)N3CCN(c4cc(Cl)cc(C(F)(F)F)c4)CC3)ccc21] |
| 41 | 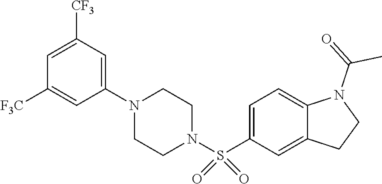 [SMILES: CC(=O)N1CCc2cc(S(=O)(=O)N3CCN(c4cc(C(F)(F)F)cc(C(F)(F)F)c4)CC3)ccc21] |
| 42 | 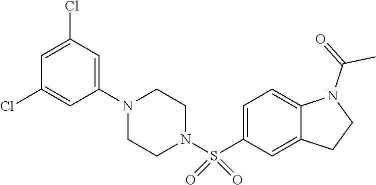 [SMILES: CC(=O)N1CCc2cc(S(=O)(=O)N3CCN(c4cc(Cl)cc(Cl)c4)CC3)ccc21] |

In another embodiment, the compounds of Formula (I) and/or salts thereof may have a structure in accordance with one of:

| Compound | |
|---|---|
| No. | Structure |
| 14 | 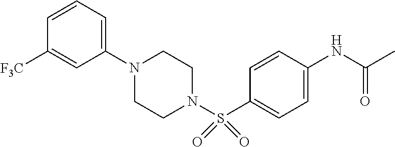 [SMILES: CC(=O)Nc1ccc(S(=O)(=O)N2CCN(c3cccc(C(F)(F)F)c3)CC2)cc1] |
| 15 | 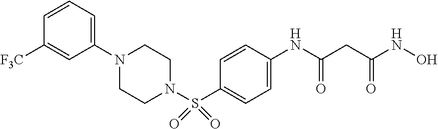 [SMILES: O=C(CC(=O)Nc1ccc(S(=O)(=O)N2CCN(c3cccc(C(F)(F)F)c3)CC2)cc1)NO] |
| 16 | 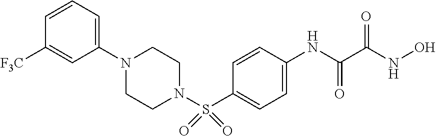 [SMILES: O=C(NO)C(=O)Nc1ccc(S(=O)(=O)N2CCN(c3cccc(C(F)(F)F)c3)CC2)cc1] |
| 25 | 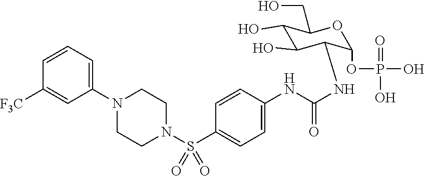 [SMILES: O=C(Nc1ccc(S(=O)(=O)N2CCN(c3cccc(C(F)(F)F)c3)CC2)cc1)NC1C(OP(=O)(O)O)OC(CO)C(O)C1O] |
| 27 | 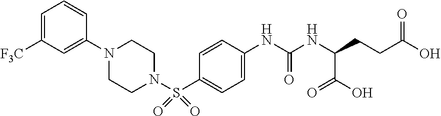 [SMILES: O=C(O)CC[C@H](NC(=O)Nc1ccc(S(=O)(=O)N2CCN(c3cccc(C(F)(F)F)c3)CC2)cc1)C(=O)O] |
| 28 | 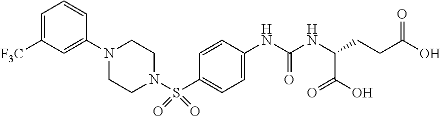 [SMILES: O=C(O)CC[C@@H](NC(=O)Nc1ccc(S(=O)(=O)N2CCN(c3cccc(C(F)(F)F)c3)CC2)cc1)C(=O)O] |
| 29 | 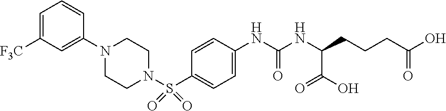 [SMILES: O=C(O)CCC[C@H](NC(=O)Nc1ccc(S(=O)(=O)N2CCN(c3cccc(C(F)(F)F)c3)CC2)cc1)C(=O)O] |
| 30 | 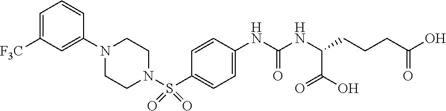 [SMILES: O=C(O)CCC[C@@H](NC(=O)Nc1ccc(S(=O)(=O)N2CCN(c3cccc(C(F)(F)F)c3)CC2)cc1)C(=O)O] |
| 33 | 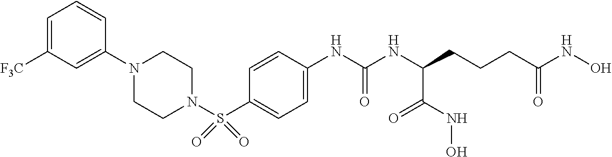 [SMILES: O=C(CCC[C@H](NC(=O)Nc1ccc(S(=O)(=O)N2CCN(c3cccc(C(F)(F)F)c3)CC2)cc1)C(=O)NO)NO] |
| 34 | 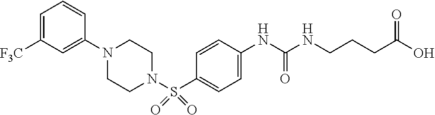 [SMILES: O=C(O)CCCNC(=O)Nc1ccc(S(=O)(=O)N2CCN(c3cccc(C(F)(F)F)c3)CC2)cc1] |
| 35 | 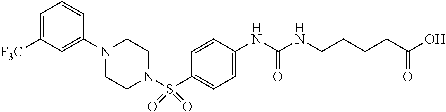 [SMILES: O=C(O)CCCCNC(=O)Nc1ccc(S(=O)(=O)N2CCN(c3cccc(C(F)(F)F)c3)CC2)cc1] |
| 36 | 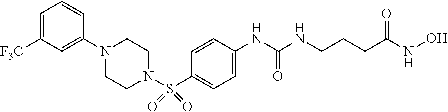 [SMILES: O=C(CCCNC(=O)Nc1ccc(S(=O)(=O)N2CCN(c3cccc(C(F)(F)F)c3)CC2)cc1)NO] |
| 37 | 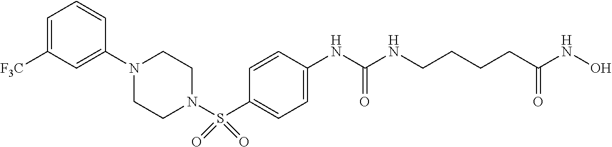 [SMILES: O=C(CCCCNC(=O)Nc1ccc(S(=O)(=O)N2CCN(c3cccc(C(F)(F)F)c3)CC2)cc1)NO] |
| 38 | 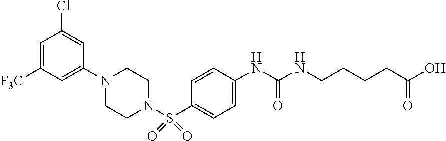 [SMILES: O=C(O)CCCCNC(=O)Nc1ccc(S(=O)(=O)N2CCN(c3cc(Cl)cc(C(F)(F)F)c3)CC2)cc1] |
| 39 | 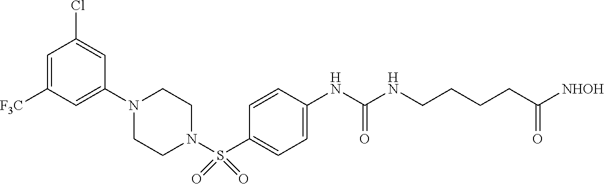 |

One of ordinary skill would understand how to make and utilize the compounds of Formula (I) based on the disclosure herein, including the exemplary synthesis procedures in the examples, and common knowledge within the field of endevour. Nevertheless, an exemplary procedure for synthesizing certain compounds of Formula (I) is provided in the following reaction scheme.

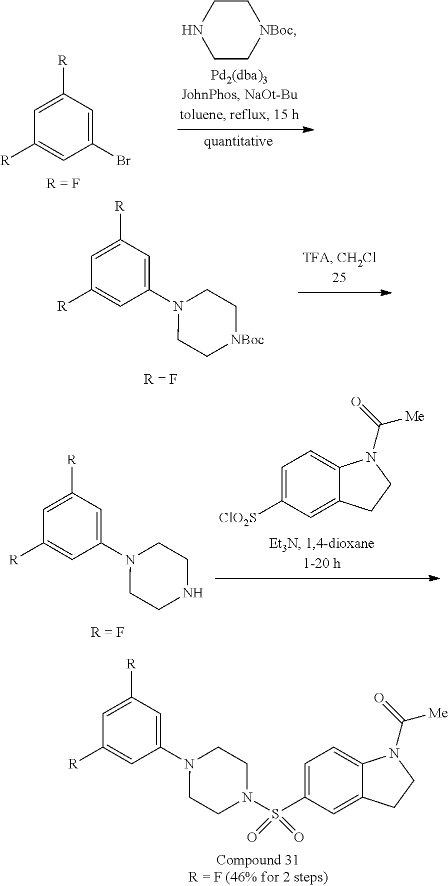

In a further aspect of the disclosure, provided are compounds of Formula (V) and/or salts thereof:

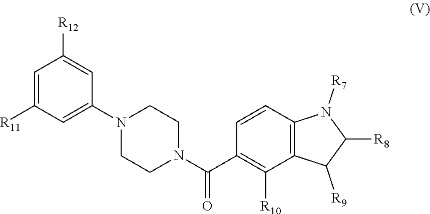

wherein,
   R₇ is hydrogen, —COCH₃, —CO₂CH₃, —COOH, —CCOH, —COH, an unsubstituted C₁ to C₅ alkyl, a C₁ to C₅ alkyl substituted with an aldehyde group, a ketone group, or a carboxylic acid group;
   R₈ and R₉ are each independently hydrogen, C₁ to C₅ substituted or unsubstituted alkyl, or taken together as a double bond; and
   R₁₁ and R₁₂ are each independently hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, C₁ to C₁₀ unsubstituted or substituted alkyl, or a combination thereof.

R₇ is typically hydrogen, —COCH₃, —CO₂CH₃, —COOH, —CCOH, —COH, an unsubstituted C₁ to C₅ alkyl, a C₁ to C₅ alkyl substituted with an aldehyde group, a ketone group, or a carboxylic acid group. In some cases, R₇ is a C₁ to C₅ unsubstituted or substituted alkyl, such as those chosen from C₁ to C₅ alkyls, C₁ to C₄ alkyls, C₁ to C₃ alkyls, C₁ to C₂ alkyls, C₂ to C₅ alkyls, C₂ to C₄ alkyls, C₂ to C₃ alkyls, C₃ to C₅ alkyls, C₃ to C₄ alkyls, and C₄ to C₅ alkyls. For example, R₇ may be a methyl, ethyl, n-propyl, i-propyl, n-butyl, or t-butyl. The C₁ to C₅ alkyl may be substituted with alcohol group (—OH), aldehyde group (—COH), ketone group (—COC), carboxylic acid group (—COOH), or a combination thereof. Preferably, R₇ is a hydrogen, —COH, —COC, or —COOH. R₇ of Formula (V) may be the same as R₂ of Formula (II).

R₈ and R₉ may independently be hydrogen, C₁ to C₅ substituted or unsubstituted alkyl, or taken together as a double bond. R₈ and R₉ may independently be a C₁ to C₅ unsubstituted or substituted alkyl, such as those chosen from C₁ to C₅ alkyls, C₁ to C₄ alkyls, C₁ to C₃ alkyls, C₁ to C₂ alkyls, C₂ to C₅ alkyls, C₂ to C₄ alkyls, C₂ to C₃ alkyls, C₃ to C₅ alkyls, C₃ to C₄ alkyls, or C₄ to C₅ alkyls. For example, R₈ and R₉ may independently be a methyl, ethyl, n-propyl, i-propyl, n-butyl, or t-butyl. The C₁ to C₅ alkyl may be substituted with alcohol group (—OH), aldehyde group (—COH), ketone group (—COC), carboxylic acid group (—COOH), or a combination thereof. In some embodiments, R₈ and R₉ may both be hydrogen or taken together as a double bond. R₈ and R₉ of Formula (V) may be the same as R₃ and R₄ of Formula (II).

R₁₁ and R₁₂ may independently be hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, C₁ to C₁₀ unsubstituted or substituted alkyl, or a combination thereof. For instance, R₁₁ and R₁₂ may independently be chosen from a fluorine, chlorine, bromine, —CF₃, and a combination thereof. R₁₁ and R₁₂ may independently be chosen from the C₄ to C₁₀ unsubstituted aryl, the C₄ to C₁₀ substituted aryl, the unsubstituted or substituted four to ten member heterocycle ring, the C₁ to C₁₀ unsubstituted alkyl, and the C₁ to C₁₀ substituted alkyl described herein with reference to R_{c} of Formula (I).

In at least one embodiment, the compound of Formula (V) has a structure in accordance with the following compound:

| Compound | |
|---|---|
| No. | Structure |
| 43 | 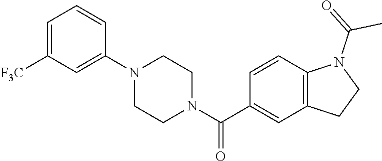 [SMILES: CC(=O)N1CCc2cc(C(=O)N3CCN(c4cccc(C(F)(F)F)c4)CC3)ccc21] |

One of ordinary skill would understand how to make and utilize the compounds of Formula (V) based on the disclosure herein and the following exemplary reaction scheme for synthesizing certain compounds of Formula (5):

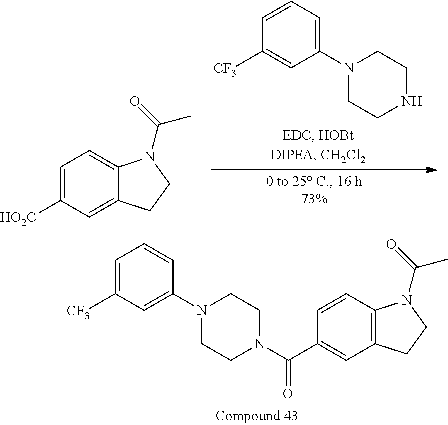

In another aspect of the disclosure, provided are compositions comprising a compound of Formula (I), as disclosed herein, and a pharmaceutical expedient. The compositions comprising a compound of Formula (I) may further include permeability enhancer. As used herein, the term “permeability enhancer” refers to any compound, drug, agent, etc. that is capable of combining with cell membranes and disrupting normal permeability to small molecules. In some embodiments, the permeability enhancer is capabable of inducing pore formation in bacterial walls. Non-limiting examples permeability enhancer include, but are not limited to, polymyxin B, DAPB, Deacylcolistin, polymyxin B nonapeptide (PMßN), Colistin nonapeptide, Polymyxin B octapeptide, Polymyxin B heptapeptide, Linear lysyl PMßN, Linear arginyl DAPB, Tetralysine, Pentalysine, Lysine₂₀, Lysine₅₀, or combinations thereof. In some embodiments, the permeability enhancer comprises PMßN.

Preferably, the composition comprises a therapeutically effective amount of the compound of Formula (I). As used herein “therapeutically effective amount” or “therapeutically effective dosage” refers to an amount that is effective to achieve a desired therapeutic result, such as lessening in the severity of the symptoms associated with a bacterial infection, decreasing the number of bacteria in the affected tissue, and/or preventing bacteria in the affected tissue from increasing in number.

In some embodiments, the desired therapeutic result is a reduction Gram-negative bacteria. For example, the therapeutically effective amount may be an amount that reduces gram-negative bacteria by at least 50% or more, preferably 60% or more, preferably 70% or more, preferably 80% or more, preferably 90% or more, preferably 95% or more, preferably 97% or more, preferably 99% or more, preferably 99.5% or more, or preferably 99.9% or more during in vitro experimental testing with Pseudomonas aeruginosa, Acinetobacter baumannii, Klebsiella pneumoniae, Escherichia coli, Haemophilius influenzae, and/or Neisseria gonorrhoeae.

In some cases, the amount of compound of Formula (I) present in composition is more than about 1 μg. For example, the composition may comprise an amount of compounds of Formula (I) and/or salts thereof of about 2 μg or more, about 5 μg or more, about 10 μg or more, about 100 μg or more, about 500 μg or more, about 1000 μg or more, about 1500 μg or more, about 2000 μg or more, about 2500 μg or more, about 3000 μg or more, about 3500 μg or more, about 4000 μg or more, about 4500 μg or more, about 5000 μg or more, about 5500 μg or more, about 6000 μg or more, about 6500 μg or more, about 7000 μg or more, about 7500 μg or more, about 8000 μg or more, about 8500 μg or more, about 9000 μg or more, about 9500 μg or more, about 10 mg or more, about 20 mg or more, about 30 mg or more, about 40 mg or more, about 50 mg or more, about 60 mg or more, about 70 mg or more, about 80 mg or more, about 90 mg or more, about 100 mg or more, about 150 mg or more, about 200 mg or more, about 250 mg or more, about 300 mg or more, about 350 mg or more, about 400 mg or more, about 450 mg or more, about 500 mg or more, about 550 mg or more, about 600 mg or more, about 650 mg or more, about 700 mg or more, about 800 mg or more, about 900 mg or more, or about 1 g or more.

Additionally or alternatively, the amount of compounds of Formula (I) and/or salts thereof present in the composition may be about 0.1 wt. % to about 95 wt. %, about 1 wt. % to about 95 wt. %, about 5 wt. % to about 95 wt. %, about 10 wt. % to about 95 wt. %, about 15 wt. % to about 95 wt. %, about 20 wt. % to about 95 wt. %, about 30 wt. % to about 95 wt. %, about 40 wt. % to about 95 wt. %, about 50 wt. % to about 95 wt. %, about 60 wt. % to about 95 wt. %, about 70 wt. % to about 95 wt. %, about 80 wt. % to about 95 wt. %; about 0.1 wt. % to about 85 wt. %, about 1 wt. % to about 85 wt. %, about 5 wt. % to about 85 wt. %, about 10 wt. % to about 85 wt. %, about 15 wt. % to about 85 wt. %, about 20 wt. % to about 85 wt. %, about 30 wt. % to about 85 wt. %, about 40 wt. % to about 85 wt. %, about 50 wt. % to about 85 wt. %, about 60 wt. % to about 85 wt. %, about 70 wt. % to about 85 wt. %; about 0.1 wt. % to about 75 wt. %, about 1 wt. % to about 75 wt. %, about 5 wt. % to about 75 wt. %, about 10 wt. % to about 75 wt. %, about 15 wt. % to about 75 wt. %, about 20 wt. % to about 75 wt. %, about 30 wt. % to about 75 wt. %, about 40 wt. % to about 75 wt. %, about 50 wt. % to about 75 wt. %, about 60 wt. % to about 75 wt. %; about 0.1 wt. % to about 65 wt. %, about 1 wt. % to about 65 wt. %, about 5 wt. % to about 65 wt. %, about 10 wt. % to about 65 wt. %, about 15 wt. % to about 65 wt. %, about 20 wt. % to about 65 wt. %, about 30 wt. % to about 65 wt. %, about 40 wt. % to about 65 wt. %, about 50 wt. % to about 65 wt. %; about 0.1 wt. % to about 55 wt. %, about 1 wt. % to about 55 wt. %, about 5 wt. % to about 55 wt. %, about 10 wt. % to about 55 wt. %, about 15 wt. % to about 55 wt. %, about 20 wt. % to about 55 wt. %, about 30 wt. % to about 55 wt. %, about 40 wt. % to about 55 wt. %; about 0.1 wt. % to about 45 wt. %, about 1 wt. % to about 45 wt. %, about 5 wt. % to about 45 wt. %, about 10 wt. % to about 45 wt. %, about 15 wt. % to about 45 wt. %, about 20 wt. % to about 45 wt. %, about 30 wt. % to about 45 wt. %; about 0.1 wt. % to about 35 wt. %, about 1 wt. % to about 35 wt. %, about 5 wt. % to about 35 wt. %, about 10 wt. % to about 35 wt. %, about 15 wt. % to about 35 wt. %, about 20 wt. % to about 35 wt. %; about 0.1 wt. % to about 25 wt. %, about 1 wt. % to about 25 wt. %, about 5 wt. % to about 25 wt. %, about 10 wt. % to about 25 wt. %; about 0.1 wt. % to about 15 wt. %, about 1 wt. % to about 15 wt. %, about 5 wt. % to about 15 wt. %, about 10 wt. % to about 15 wt. %; about 0.1 wt. % to about 10 wt. %, about 1 wt. % to about 10 wt. %, or about 5 wt. % to about 10 wt. %, including ranges and subranges thereof, based on the total weight of the composition.

The compositions of compounds of Formula (I) and/or salts thereof typically include at least one pharmaceutically acceptable excipient. Non-limiting examples of pharmaceutically acceptable excipients include a diluent, a binder, a filler, a buffering agent, a pH modifying agent, a disintegrant, a dispersant, a preservative, a lubricant, taste-masking agent, a flavoring agent, a coloring agent, or a combination thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate, or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

The compositions disclosed herein may be formulated into various dosage forms and administered by a number of different means that will deliver a therapeutically effective amount of the active ingredient. Such compositions may be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term, “parenteral,” as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18th ed., 1995), and Liberman, H. A. and Lachman, L., Eds., Pharmaceutical Dosage Forms, Marcel Dekker Inc., New York, N.Y. (1980). In a specific embodiment, the composition may be a food supplement or a cosmetic.

Solid dosage forms for oral administration may be contained in capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions disclosed herein may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions of compounds of Formula (I) and/or salts thereof adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions of compounds of Formula (I) and/or salts thereof adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising compounds of Formula (I) and/or salts thereof is encapsulated in a suitable vehicle to either aid in the delivery of the compound of Formula (I) to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of a composition comprising compounds of Formula (I) and/or salts thereof or a salt of the compound of Formula (I) in view of their structural and chemical properties. Generally, liposomes are spherical vesicles with a phospholipid bilayer membrane. The lipid bilayer of a liposome may fuse with other bilayers (e.g., the cell membrane), thus delivering the contents of the liposome to cells.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a composition comprising the compound of Formula (I) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211, and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration, and composition of lipid, concentration of multivalent cations, rate of mixing, presence of and concentration of solvent.

The composition may be formulated as part of a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. A composition comprising at least one anti-viral therapeutic derivative may be encapsulated in a microemulsion by any method generally known in the art.

In yet another embodiment, the composition comprising compounds of Formula (I) and/or salts thereof or a salt thereof may be delivered in a dendritic macromolecule, or a dendrimer. Generally, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

The compositions comprising the compound(s) of Formula (I) or salts thereof may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. The compounds of Formula (I) may be formulated in the compositions disclosed herein per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Aspects of the disclosure also relate to methods for treating a Gram-negative bacterial infection in a patient by administering a compound of compounds of Formula (I), salts thereof, and/or compositions thereof to a patient in need of treatment thereof. In at least one embodiment, the method further comprises administrating the subject a therapeutically effective amount of an outer membrane permeability enhancer. In some embodiments, the permeability enhance is administered prior to the administration of the one or more compounds of Formula (I) and/or compositions thereof. In other embodiments, the permeability enhancer is administered concurrently with the one or more compounds of Formula (I), salts thereof, and/or compositions thereof. In yet other embodiments, the permeability enhancer is administered after the administration of the one or more compounds of Formula (I), salts thereof, and/or compositions thereof.

The methods of the disclosure may include administering an amount of a compound of Formula (I) or compositions thereof topically, orally, or parenterally. For topical administration, the dosage of composition may be in the form of a cream, a serum, a lotion, a gel, or the like. In at least one case, the dosage of the compositions disclosed herein contained in the gel of an adhesive gel patch.

For oral administration, the method may include administering an amount of the composition in the form of a solid dosage or a liquid dosage. Solid dosage forms for oral administration include capsules, tablets, caplets, pills, powders, pellets, and granules. In such solid dosage forms, the compounds of Formula (I) and/or salts thereof are, typically, combined with one or more excipients, such as those described above. Liquid dosages of the composition disclosed herein may be in the form of aqueous suspensions, elixirs, or syrups. For these, the composition may be combined with various sweetening or flavoring agents, coloring agents, and, if so desired, emulsifying and/or suspending agents, as well as diluents such as water, ethanol, glycerin, and combinations thereof.

For parenteral administration, the dosage of composition may be an aqueous solution, an oil-based solution, or in the form of a solid dosage. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. In some instances, parental administration may be subcutaneous, intravenous, intramuscular, or intrasternal injection, or infusion.

The administration of the compounds of Formula (I) and/or salts thereof and/or salts thereof may occur on a regular period or an irregular period. For example, the method may include administration of the compositions disclosed herein once every hour, once every two hours, once every three hours, once every four hours, once every five hours, once every six hours, once every seven hours, once every eight hours, etc. The period of time during which the therapeutically effective dosage formulation comprising a therapeutically effective amount of composition is administered can and will vary depending on a number of factors, including the severity of disorder being treated, the type of formulation, route and frequency of administration, the age of the subject, etc. Generally, the period of administration is at least several times a day and/or a week.

The method may include dosage amounts in the range of from about 0.0001 mg/kg/day, 0.001 mg/kg/day or 0.01 mg/kg/day to about 100 mg/kg/day, or 1 g/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) of Formula (I) and/or active metabolite compound(s) of Formula (I), which are sufficient to maintain therapeutic or prophylactic effect.

As used herein, “compounds of Formula (I)”, “Formula (I),” “formula (I)” and “compounds of the disclosure” are being used interchangeably throughout the application and should be treated as synonyms. The references to compounds of Formula (I) herein may be in salt form without deviating from the scope or spirit of the invention. In some cases, however, the compounds of Formula (I) may not be suitable in a salt form and, thus, may be excluded.

“Substituted” refers to a group having one or more hydrogens replaced with substituents. The substituents may be one or more of acyl alkyl, alkoxy, allyl, alkenyl, alkynyl, aromatic, aryl, carbocyclic, halogen, heteroaromatic, hydrocarbon, acylamino, acyloxy, amino, aminocarbonyl, aminothiocarbonyl, aminocarbonylamino, aminothiocarbonylamino, aminocarbonyloxy, amidino, carboxyl, carboxyl ester, (carboxyl ester)amino, (carboxyl ester)oxy, cyano, guanidino, halo, hydroxy, nitro, SO₃H, sulfonyl, sulfonyloxy, thioacyl, thiol, and alkylthio, wherein said substituents are as defined herein. In certain substituted cyclic groups, “substituted” also refers to a group having two hydrogens replaced with a single double bonded oxygen atom (an oxo group) or a single double bonded sulfur atom (thioxo). In some implementations, the substituted group has 1 to 3 of the aforementioned substituents. In other implementations, the substituted group has 1 to 2 of the aforementioned substituents. In further implementations, the substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The term “acyl,” alone or as part of another group, denotes the moiety formed by removal of the hydroxy group from the group COOH of an organic carboxylic acid, e.g., RC(O)—, wherein R is R^{X}, R^{X}O—, R^{X}R^{Y}N—, or R^{X}S—, R^{X} is hydrocarbyl, heterosubstituted hydrocarbyl, or heterocyclo, and R^{Y} is hydrogen, hydrocarbyl, or substituted hydrocarbyl. Additionally, acyl may refer to branched or straight chained alkyl group.

The term “acyloxy,” as used herein, alone or as part of another group, denotes an acyl group as described above bonded through an oxygen linkage (O), e.g., RC(O)O— wherein R is as defined in connection with the term “acyl.” Additionally, acyloxy may refer to branched or straight chained acyloxy group.

The term “allyl,” as used herein, not only refers to compound containing the simple allyl group (CH₂═CH—CH₂—), but also to compounds that contain substituted allyl groups or allyl groups forming part of a ring system. Additionally, allyl may refer to branched or straight chained allyl group.

The term “alkyl,” as used herein, describes groups that are preferably lower alkyl containing from one to eight carbon atoms in the principal chain and up to 20 carbon atoms. Additionally, alkyl may refer to straight, branched chain, or cyclic chained alkyl group, and may include methyl, ethyl, propyl, isopropyl, butyl, hexyl and the like.

The term “alkenyl,” as used herein, describes groups that are preferably lower alkenyl containing from two to eight carbon atoms in the principal chain and up to 20 carbon atoms. They may be straight, branched chain, cyclic and include ethenyl, propenyl, isopropenyl, butenyl, isobutenyl, hexenyl, and the like.

The term “alkynyl,” as used herein, describes groups that are preferably lower alkynyl containing from two to eight carbon atoms in the principal chain and up to 20 carbon atoms. They may be straight or branched chain and include ethynyl, propynyl, butynyl, isobutynyl, hexynyl, and the like.

The term “aromatic,” as used herein, alone or as part of another group denotes optionally substituted homo- or heterocyclic conjugated planar ring or ring system comprising delocalized electrons. These aromatic groups are preferably monocyclic (e.g., furan or benzene), bicyclic, or tricyclic groups containing from 5 to 14 atoms in the ring portion. The term “aromatic” encompasses “aryl” groups defined below.

The terms “aryl” or “Ar,” as used herein, alone or as part of another group denote optionally substituted homocyclic aromatic groups, preferably monocyclic or bicyclic groups containing from 6 to 10 carbons in the ring portion, such as phenyl, biphenyl, naphthyl, substituted phenyl, substituted biphenyl, or substituted naphthyl.

The terms “carbocyclo” or “carbocyclic,” as used herein, alone or as part of another group denote optionally substituted, aromatic or non-aromatic, homocyclic ring or ring system in which all of the atoms in the ring are carbon, with preferably 5 or 6 carbon atoms in each ring. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “halogen” or “halo,” as used herein, alone or as part of another group refer to chlorine, bromine, fluorine, and iodine.

The term “heteroatom,” as used herein, refers to atoms other than carbon and hydrogen.

The term “heteroaromatic,” as used herein, alone or as part of another group denotes optionally substituted aromatic groups having at least one heteroatom in at least one ring, and preferably 5 or 6 atoms in each ring. The heteroaromatic group may have 1 or 2 oxygen atoms and/or 1 to 4 nitrogen atoms in the ring, and is bonded to the remainder of the molecule through a carbon. For example, it may refer to a 5- or 6-membered ring containing 1, 2, 3, or 4 nitrogen atoms; 1 oxygen atom; 1 sulfur atom; 1 nitrogen and 1 sulfur atom; 1 nitrogen and 1 oxygen atom; 2 nitrogen atoms and 1 oxygen atom; or 2 nitrogen atoms and 1 sulfur atom. The 5-membered ring has 2 double bonds and the 6-membered ring has 3 double bonds. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “heterocyclo” or “heterocyclic,” as used herein, alone or as part of another group denote optionally substituted, fully saturated or unsaturated, monocyclic or bicyclic, aromatic or non-aromatic groups having at least one heteroatom in at least one ring, and preferably 5 or 6 atoms in each ring. The heterocyclo group may have 1 or 2 oxygen atoms and/or 1 to 4 nitrogen atoms in the ring, and is bonded to the remainder of the molecule through a carbon or heteroatom. Exemplary heterocyclo groups include heteroaromatics as described above. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxy, keto, ketal, phospho, nitro, and thio.

The terms “hydrocarbon” and “hydrocarbyl,” as used herein, refer to organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “patient” refers to warm blooded animals such as, for example, guinea pigs, mice, rats, gerbils, cats, rabbits, dogs, monkeys, chimpanzees, and humans.

The term “treat” may refer to the ability of the compounds to relieve, alleviate or slow the progression of the patient's bacterial infection (or condition) or any tissue damage associated with the disease.

Compounds of the disclosure if containing an asymmetrically substituted atom may be isolated in optically active or racemic form. All chiral, diastereomeric, racemic forms and all geometric isomeric forms of a structure are intended, unless the specific stereochemistry or isomeric form is specifically indicated. Geometric isomeric forms refers to compounds that may exist in cis, trans, anti, entgegen (E), and zusammen (Z) forms as well as mixtures thereof.

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

All components, elements, features and substituents positively set forth in this disclosure can be negatively excluded or omitted from the claims. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As various changes could be made in the above-described methods without departing from the scope of the invention, it is intended that all matter contained in the above description and in the examples given below, shall be interpreted as illustrative and not in a limiting sense.

### EXAMPLES

The following non-limiting examples of aspects of the invention are provided primary for the purpose of elucidating aspects of the invention and the benefits derived therefrom.

### Example 1: Synthesis of sulfonyl piperazine LpxH inhibitors

General chemistry procedures. All of the reactions to synthesis sulfonyl piperazine LpxH inhibitors were conducted in oven-dried glassware under nitrogen or argon. The reagents were purchased from commercial suppliers and used without further purification unless otherwise stated. The solvents were American Chemical Society (ACS) grade or better and were used without further purification, except for tetrahydrofuran (THF), which was freshly distilled from sodium/benzophenone each time before use. Analytical thin layer chromatography (TLC) was performed with glass backed silica gel (60 Å) plates with fluorescent indication (Whatman). Visualization was accomplished by ultra violet (UV) irradiation at 254 nm and/or by staining with p-anisaldehyde solution. Flash column chromatography was performed by using silica gel (particle size 230-400 mesh, 60 Å). All ¹H spectra were recorded with a Varian 400 spectrometer. All ¹H NMR δ values are given in parts per million (ppm) and are referenced to the residual solvent signals (CDCl₃: δ=7.26 ppm, CD₃OD: δ=3.31 ppm, CD₃COCD₃: δ=2.05 ppm). Coupling constants (J) are given in Hertz (Hz) and multiplicities are indicated using the conventional abbreviation (s=singlet, d=doublet, t=triplet, q=quartet, m=multiplet or overlap of non-equivalent resonances, br=broad). Electrospray ionization (ESI) mass spectrometry (MS) was recorded with an Agilent 1100 series (LC/MSD trap) spectrometer in order to obtain the molecular masses of compounds. Optical rotation values were measured with a Rudolph Research Analytical (A21102 API/1W) polarimeter. The purity of final compounds used in bioassays was determined by NMR and was found to be >95%.

### Example 1A

tert-Butyl 4-(3-bromo-5-(trifluoromethyl)phenyl)piperazine-1-carboxylate, the structure of which is shown below, was synthesized according to the following procedure.

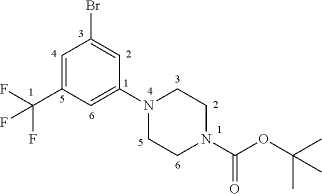 [SMILES: CC(C)(C)OC(=O)N1CCN(c2cc(Br)cc(C(F)(F)F)c2)CC1]

Anhydrous toluene (4.8 mL) was added to a mixture of 1,3-dibromo-5-(trifluoromethyl)benzene (500 mg, 1.6 mmol), 1-Boc-piperazine (596 mg, 3.2 mmol), NaOt-Bu (307 mg, 3.2 mmol), JohnPhos (71.6 mg, 0.24 mmol), and Pd₂(dba)₃ (68.6 mg, 0.08 mmol). Argon (Ar) was bubbled through the reaction mixture for 15 min before the reaction mixture was heated to reflux for 14 hours (h). The reaction mixture was concentrated in vacuo, dissolved in CH₂Cl₂/MeOH (1/1), and filtered through a pad of Celite. The filtrate was concentrated in vacuo and purified by column chromatography (silica gel, hexanes/EtOAc, 5/1) to afford N-aryl piperazine (100 mg, 14%) as a yellow solid: ¹H NMR (400 MHz, CDCl₃) δ 7.20 (s, 1H), 7.15 (s, 1H), 7.01 (s, 1H), 3.61-3.53 (m, 4H), 3.23-3.15 (m, 4H), 1.48 (s, 9H); HRMS (ESI) m/z 431.0545 [(M+Na)⁺ calcd for C₁₆H₂₀BrF₃N₂O₂ 431.0553].

### Example 1B

1-(3-Bromo-5-(trifluoromethyl)phenyl)piperazine, the structure of which is shown below, was synthesized according to the following procedure.

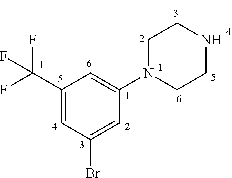 [SMILES: FC(F)(F)c1cc(Br)cc(N2CCNCC2)c1]

To a cooled (0° C.) solution of Tert-Butyl 4-(3-bromo-5-(trifluoromethyl)phenyl)piperazine-1-carboxylate (100 mg, 0.22 mmol) in anhydrous CH₂Cl₂ (1 mL) was added dropwise TFA (0.5 mL). It appears that After stirring at 25° C. for 2.5 h, the solvents were removed under reduced pressure to give 1-(3-Bromo-5-(trifluoromethyl)phenyl)piperazine (100 mg) as an orange solid. 1-(3-Bromo-5-(trifluoromethyl)phenyl)piperazine compounds were used in the following step without further purification: ¹H NMR (400 MHz, CDCl₃) δ 9.35 (br s, 1H), 7.34 (s, 1H), 7.21 (s, 1H), 7.05 (s, 1H), 3.50 (t, J=5.1 Hz, 4H), 3.45-3.36 (m, 4H); HRMS (ESI) m/z 309.0216 [(M+H)⁺ calcd for C₁₁H₁₂BrF₃N₂ 309.0209].

### Example 1C

1-(5-((4-(3-Bromo-5-(trifluoromethyl)phenyl)piperazin-1-yl)sulfonyl)indolin-1-yl)ethan-1-one, the structure of which is shown below, was synthesized according to the following procedure.

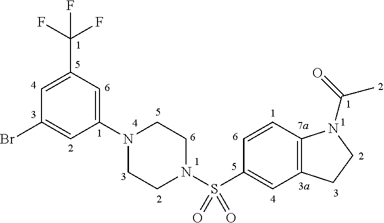 [SMILES: CC(=O)N1CCc2cc(S(=O)(=O)N3CCN(c4cc(Br)cc(C(F)(F)F)c4)CC3)ccc21]

A solution of 1-(3-Bromo-5-(trifluoromethyl)phenyl)piperazine (100 mg, 0.32 mmol) and Et₃N (50 μL, 0.38 mmol) in anhydrous 1,4-dioxane (0.22 M, 1.5 mL) was heated to 60° C. 1-Acetylindoline-5-sulfonyl chloride (41.5 mg, 0.16 mmol) in anhydrous 1,4-dioxane (1 mL) was added to the reaction mixture. After stirring at 60° C. for 3 h, the reaction mixture was cooled to 25° C. The reaction was quenched by an addition of H₂O and the resulting mixture was extracted with EtOAc. The combined organic layers were washed with brine, dried over anhydrous Na₂SO₄, and concentrated in vacuo. The residue was purified by column chromatography (silica gel, hexanes/EtOAc, 1/1) to afford 1-(5-((4-(3-Bromo-5-(trifluoromethyl)phenyl)piperazin-1-yl)sulfonyl)indolin-1-yl)ethan-1-one (37 mg, 43% for 2 steps) as a white solid: ¹H NMR (400 MHz, CDCl₃) δ 8.34 (d, J=8.5 Hz, 1H), 7.63 (d, J=8.4 Hz, 1H), 7.57 (s, 1H), 7.21 (s, 1H), 7.09 (s, 1H), 6.95 (s, 1H), 4.16 (t, J=8.6 Hz, 2H), 3.34-3.24 (m, 6H), 3.17-3.11 (m, 4H), 2.27 (s, 3H); HRMS (ESI) m/z 532.0517 [(M+H)⁺ calcd for C₂₁H₂₁BrF₃N₃O₃S 532.0512].

### Example 1D

tert-Butyl 4-(3-methyl-5-(trifluoromethyl)phenyl)piperazine-1-carboxylate, the structure of which is shown below, was synthesized according to the following procedure.

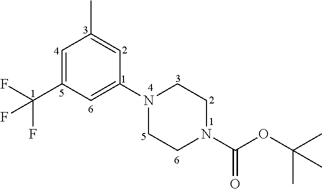 [SMILES: Cc1cc(N2CCN(C(=O)OC(C)(C)C)CC2)cc(C(F)(F)F)c1]

To a solution of 1-bromo-3-methyl-5-(trifluoromethyl)benzene (50 mg, 0.21 mmol), 1-Boc-piperazine (51 mg, 0.27 mmol), and NaOt-Bu (30 mg, 0.31 mmol) in toluene (0.63 mL) was added JohnPhos (6.3 mg, 0.02 mmol, 10 mol %), and Pd₂(dba)₃ (9.2 mg, 0.01 mmol, 5 mol %). Argon (Ar) was bubbled through the reaction mixture for 15 minutes (min) and then the reaction was heated to reflux for 15 h. The reaction mixture was concentrated in vacuo, dissolved in CH₂Cl₂/MeOH (1/1), and filtered through a pad of Celite. The residue was purified by column chromatography (silica gel, hexanes/EtOAc, 20/1) to afford tert-Butyl 4-(3-methyl-5-(trifluoromethyl)phenyl)piperazine-1-carboxylate (58 mg, 81%): ¹H NMR (400 MHz, CDCl₃) δ 6.92 (s, 2H), 6.87 (s, 1H), 3.62-3.51 (m, 4H), 3.21-3.08 (m, 4H), 2.35 (s, 3H), 1.47 (s, 9H).

### Example 1E

1-(3-Methyl-5-(trifluoromethyl)phenyl)piperazine, the structure of which is shown below, was synthesized according to the following procedure.

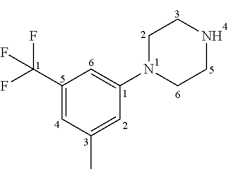 [SMILES: Cc1cc(N2CCNCC2)cc(C(F)(F)F)c1]

TFA (0.33 mL) was added to a solution of tert-Butyl 4-(3-methyl-5-(trifluoromethyl)phenyl)piperazine-1-carboxylate (58 mg, 0.17 mmol) in CH₂Cl₂ (0.85 mL). The reaction mixture was stirred at 25° C. for 2 h and then was concentrated in vacuo to afford 1-(3-Methyl-5-(trifluoromethyl)phenyl)piperazine (41 mg). 1-(3-Methyl-5-(trifluoromethyl)phenyl)piperazine compounds were used in the following step without further purification: ¹H NMR (400 MHz, CDCl₃) δ 7.03 (s, 1H), 6.93 (s, 1H), 6.88 (s, 1H), 3.60-3.46 (m, 4H), 2.58-2.4 (m, 4H), 2.37 (s, 3H).

### Example 1F

1-(5-((4-(3-Methyl-5-(trifluoromethyl)phenyl)piperazin-1-yl)sulfonyl)indolin-1-yl)ethan-1-one, the structure of which is shown below, was synthesized according to the following procedure.

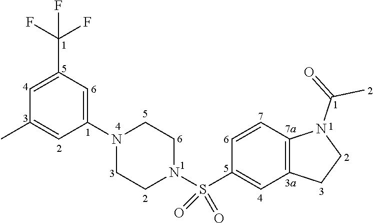 [SMILES: CC(=O)N1CCc2cc(S(=O)(=O)N3CCN(c4cc(C)cc(C(F)(F)F)c4)CC3)ccc21]

A solution of 1-(3-Methyl-5-(trifluoromethyl)phenyl)piperazine (27 mg, 0.11 mmol) and Et₃N (17 μL, 0.13 mmol) in 1,4-dioxane (1.63 mL) was heated to 60° C. To this solution, 1-acetylindoline-5-sulfonyl chloride (29 mg, 0.11 mmol) was added and the resulting mixture was stirred at 60° C. for 3 h followed by at 25° C. for 14 h. The reaction was quenched by an addition of H₂O and the aqueous layer was extracted with EtOAc. The combined organic layers were dried over anhydrous Na₂SO₄ and concentrated in vacuo. The crude product was purified by column chromatography (silica gel, hexanes/EtOAc, 2/1) to afford 1-(5-((4-(3-Methyl-5-(trifluoromethyl)phenyl)piperazin-1-yl)sulfonyl)indolin-1-yl)ethan-1-one (26.3 mg, 51% for 2 steps): ¹H NMR (400 MHz, CDCl₃) δ 8.33 (d, J=7.9 Hz, 1H), 7.62 (d, J=7.9 Hz, 1H), 7.56 (s, 1H), 6.93 (s, 1H), 6.85 (s, 1H), 6.82 (s, 1H), 4.14 (t, J=8.6 Hz, 2H), 3.31-3.22 (m, 6H), 3.18-3.10 (m, 4H), 2.32 (s, 3H), 2.25 (s, 3H); HRMS (ESI) m/z 468.1554 [(M+H)⁺ calcd for C₂₂H₂₄F₃N₃O₃S 468.1563].

### Example 1G

tert-Butyl 4-(3,5-bis(trifluoromethyl)phenyl)piperazine-1-carboxylate, the structure of which is shown below, was synthesized according to the following procedure.

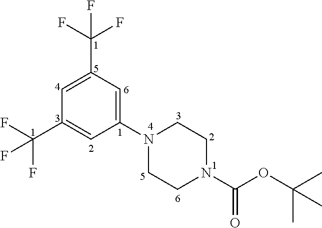 [SMILES: CC(C)(C)OC(=O)N1CCN(c2cc(C(F)(F)F)cc(C(F)(F)F)c2)CC1]

Toluene (1.6 mL) was added to a mixture of 1,3-bis(trifluoromethyl)-5-bromobenzene (150 mg, 0.51 mmol), 1-Boc-piperazine (123 mg, 0.66 mmol), NaOt-Bu (74 mg, 0.77 mmol), JohnPhos (15 mg, 10 mol %), and Pd₂(dba)₃ (28 mg, 5 mol %). Argon (Ar) was bubbled through the reaction mixture for 30 min, and the reaction mixture was refluxed for 18 h. The reaction mixture was concentrated in vacuo and the residue was dissolved in CH₂Cl₂/MeOH (1/1), filtered through a pad of Celite, and concentrated in vacuo. The residue was purified by column chromatography (silica gel, hexanes/EtOAc, 20/1) to afford tert-butyl 4-(3,5-bis(trifluoromethyl)phenyl)piperazine-1-carboxylate (203 mg, quantitative): ¹H NMR (400 MHz, CDCl₃) δ 7.30 (s, 1H), 7.25 (s, 2H), 3.65-3.59 (m, 4H), 3.27-3.24 (m, 4H), 1.48 (s, 9H).

### Example 1H

1-(3,5-Bis(trifluoromethyl)phenyl)piperazine, the structure of which is shown below, was synthesized according to the following procedure.

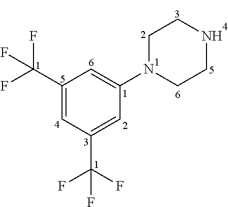 [SMILES: FC(F)(F)c1cc(N2CCNCC2)cc(C(F)(F)F)c1]

To a solution of tert-butyl 4-(3,5-bis(trifluoromethyl)phenyl)piperazine-1-carboxylate (200 mg, 0.5 mmol) in CH₂Cl₂ (3 mL), TFA (1.3 mL) was added at 0° C. The reaction mixture was stirred under N₂ at 0° C. for 10 min and then warmed to 25° C. The reaction mixture was stirred under N₂ at 25° C. for 3.5 h. The reaction mixture was concentrated in vacuo to give 1-(3,5-bis(trifluoromethyl)phenyl)piperazine as an orange solid. 1-(3,5-bis(trifluoromethyl)phenyl)piperazine compounds was used in the following step without further purification: ¹H NMR (400 MHz, CDCl₃) δ 7.45 (s, 1H), 7.30 (s, 2H), 3.95 (br s, 1H), 3.59 (m, 4H), 3.46 (m, 4H).

### Example 1I

1-(5-((4-(3,5-Bis(trifluoromethyl)phenyl)piperazin-1-yl)sulfonyl)indolin-1-yl)ethan-1-one (Compound 41), the structure of which is shown below, was synthesized according to the following procedure.

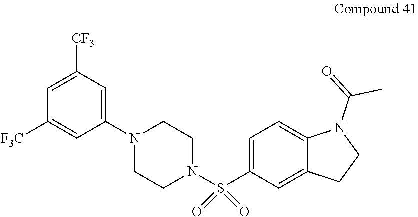 [SMILES: CC(=O)N1CCc2cc(S(=O)(=O)N3CCN(c4cc(C(F)(F)F)cc(C(F)(F)F)c4)CC3)ccc21]

Et₃N (32 μL, 0.24 mmol) was added to a solution of 1-(3,5-bis(trifluoromethyl)phenyl)piperazine (50 mg, 0.17 mmol) in 1,4-dioxane (3 mL) at 25° C. 1-acetyl-5-indoline sulfonyl chloride (44 mg, 0.17 mmol) was added to the reaction mixture. The resulting mixture was stirred under N₂ at 60° C. for 3 h. The reaction mixture was cooled to 25° C. and kept at the same temperature for 10 h. The reaction mixture was diluted with EtOAc. The layers were separated, and the aqueous layer was extracted with EtOAc. The combined organic layers were washed with brine, dried over anhydrous Na₂SO₄, and concentrated under reduced pressure. The residue was purified by column chromatography (silica gel, hexanes/EtOAc, 4/1) to afford Compound 41 as a white solid (35 mg, 40% for 2 steps): ¹H NMR (400 MHz, CDCl₃) δ 8.34 (d, J=8.6 Hz, 1H), 7.62 (dd, J=8.5, 1.9 Hz, 1H), 7.57 (s, 1H), 7.31 (s, 1H), 7.19 (s, 2H), 4.17 (t, J=8.5 Hz, 2H), 3.36-3.34 (m, 4H), 3.28 (t, J=8.8 Hz, 3H), 3.16-3.15 (m, 4H), 2.27 (s, 3H); HRMS (ESI): m/z 522.1281 [(M+H)⁺ calcd for C₂₂H₂₁F₆N₃O₃S 522.1289].

### Example 1J

tert-Butyl 4-(3,5-difluorophenyl)piperazine-1-carboxylate, the structure of which is shown below, was synthesized according to the following procedure.

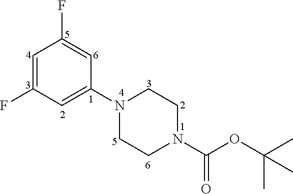 [SMILES: CC(C)(C)OC(=O)N1CCN(c2cc(F)cc(F)c2)CC1]

Toluene (4 mL) was added to a mixture of 1-bromo-3,5-difluorobenzene (250 mg, 1.3 mmol), 1-Boc-piperazine (315 mg, 1.69 mmol), NaOt-Bu (187 mg, 1.95 mmol), JohnPhos (39 mg, 10 mol %), and Pd₂(dba)₃ (60 mg, 5 mol %). Argon (Ar) was bubbled through the reaction mixture for 30 min, and the reaction mixture was refluxed for 15 h. The reaction mixture was concentrated in vacuo and the residue was dissolved in CH₂Cl₂/MeOH (1/1), filtered through a pad of Celite, and concentrated in vacuo. The residue was purified by column chromatography (silica gel, hexanes/EtOAc, 20/1) to afford tert-butyl 4-(3,5-difluorophenyl)piperazine-1-carboxylate (436 mg, quantitative) as a solid: ¹H NMR (400 MHz, CDCl₃) δ 6.36 (d, J=8.8 Hz, 2H), 6.27 (t, J=8.8 Hz, 1H), 3.60-3.52 (m, 4H), 3.20-3.11 (m, 4H), 1.48 (s, 9H).

### Example 1K

1-(3,5-Difluorophenyl)piperazine, the structure of which is shown below, was synthesized according to the following procedure.

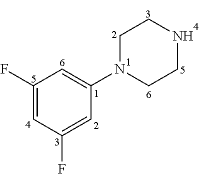 [SMILES: Fc1cc(F)cc(N2CCNCC2)c1]

TFA (1.4 mL) was added to a solution of tert-butyl 4-(3,5-difluorophenyl)piperazine-1-carboxylate (207 mg, 0.69 mmol) in CH₂Cl₂ (3.5 mL), and the resulting mixture was stirred at 25° C. for 1 h. The reaction mixture was concentrated in vacuo to afford 1-(3,5-difluorophenyl)piperazine. 1-(3,5-Difluorophenyl)piperazine compounds were used in the following step without further purification: ¹H NMR (400 MHz, CD₃OD) δ 6.61 (d, J=10.0 Hz, 2H), 6.42 (t, J=9.0 Hz, 1H), 3.48-3.45 (m, 4H), 3.36-3.33 (m, 4H); HRMS (ESI) m/z 199.1041 [(M+H)⁺ calcd for C₁₀H₁₂F₂N₂ 199.1041].

### Example 1L

1-(5-((4-(3,5-Difluorophenyl)piperazin-1-yl)sulfonyl)indolin-1-yl)ethan-1-one, the structure of which is shown below, was synthesized according to the following procedure.

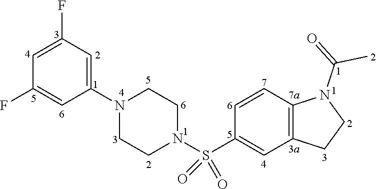 [SMILES: CC(=O)N1CCc2cc(S(=O)(=O)N3CCN(c4cc(F)cc(F)c4)CC3)ccc21]

A solution of 1-(3,5-difluorophenyl)piperazine (50 mg, 0.25 mmol) and Et₃N (40 μL, 0.3 mmol) in 1,4-dioxane (3.7 mL) was heated to 60° C. and 1-acetyl-5-indolinesulfonyl chloride (65 mg, 0.25 mmol) was added. After stirring at 60° C. for 3 h, the reaction mixture was cooled to 25° C. and stirred for additional 14 h. The reaction was quenched by an addition of H₂O. The reaction mixture was extracted with EtOAc. The combined organic layers were dried over anhydrous Na₂SO₄ and concentrated in vacuo. The residue was purified by column chromatography (silica gel, hexanes/EtOAc, 1/1) to afford 1-(5-((4-(3,5-difluorophenyl)piperazin-1-yl)sulfonyl)indolin-1-yl)ethan-1-one (48 mg, 46% for 2 steps) as a white solid: 1H NMR (400 MHz, CDCl₃) δ 8.34 (d, J=8.2 Hz, 1H), 7.62 (d, J=8.2 Hz, 1H), 7.56 (s, 1H), 6.31-6.25 (m, 3H), 4.16 (t, J=8.5 Hz, 2H), 3.30-3.26 (m, 6H), 3.12-3.11 (m, 4H), 2.26 (s, 3H); HRMS (ESI) m/z 422.1349 [(M+H)⁺ calcd for C₂₀H₂₁F₂N₃O₃S 422.1345].

### Example 1M

1-(5-((4-(3,5-Dichlorophenyl)piperazin-1-yl)sulfonyl)indolin-1-yl)ethan-1-one (Compound 42), the structure of which is shown below, was synthesized according to the following procedure.

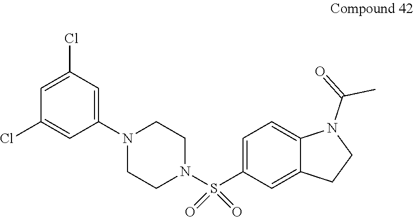 [SMILES: CC(=O)N1CCc2cc(S(=O)(=O)N3CCN(c4cc(Cl)cc(Cl)c4)CC3)ccc21]

To a solution of commercially available 1-(3-5-dichlorophenyl)piperazine (50 mg, 0.22 mmol) in 1,4-dioxane (3.0 mL), Et₃N (34 μL, 0.26 mmol) was added at 25° C. The reaction mixture was treated with 1-acetyl-5-indoline sulfonyl chloride (57 mg, 0.22 mmol) and stirred under N₂ at 60° C. for 3 h. The reaction mixture was cooled to 25° C. and then stirred for an additional 20 h. The reaction mixture was diluted with EtOAc. The layers were separated, and the aqueous layer was extracted with EtOAc. The combined organic layers were washed with brine, dried over anhydrous Na₂SO₄, and concentrated in vacuo. The residue was purified by column chromatography (silica gel, hexanes/EtOAc, 4/1) to afford Compound 42 as a white solid (46 mg, 47%): ¹H NMR (400 MHz, CDCl₃) δ 8.34 (d, J=8.5 Hz, 1H), 7.60 (dd, J=8.6, 1.9 Hz, 1H), 7.55 (s, 1H), 6.83 (t, J=1.7 Hz, 1H), 6.69 (d, J=1.8 Hz, 2H), 4.15 (t, J=8.6 Hz, 2H), 3.27-3.24 (m, 6H), 3.13-3.10 (m, 4H), 2.26 (s, 3H); HRMS (ESI): m/z 454.0753 [(M+H)⁺ calcd for C₂₀H₂₁Cl₂N₃O₃S 454.0759].

### Example 1N

5-((4-(3-Chloro-5-(trifluoromethyl)phenyl)piperazin-1-yl)sulfonyl)-1H-indole, the structure of which is shown below, was synthesized according to the following procedure.

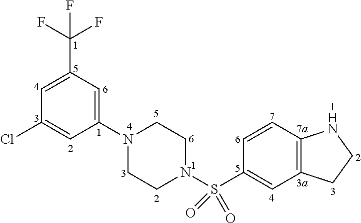 [SMILES: O=S(=O)(c1ccc2c(c1)CCN2)N1CCN(c2cc(Cl)cc(C(F)(F)F)c2)CC1]

1-(3-Chloro-5-(trifluoromethyl)phenyl)piperazine (121 mg, 0.46 mmol) in anhydrous 1,4-dioxane (0.5 mL) was added to a solution (at a temperature of 60° C.) of 1H-indole-5-sulfonyl chloride (50 mg, 0.23 mmol) and Et₃N (72 μL, 0.55 mmol) in anhydrous 1,4-dioxane (0.22 M, 1 mL). After stirring at 60° C. for 2 h, the reaction mixture was cooled to 25° C. and stirred for an additional 18 h. The reaction was quenched by an addition of H₂O and the resulting mixture was diluted with EtOAc. The combined organic layers were washed with brine, dried over anhydrous Na₂SO₄, and concentrated in vacuo. The residue was purified by column chromatography (silica gel, hexanes/EtOAc, 3/1) to afford 5-((4-(3-chloro-5-(trifluoromethyl)phenyl)piperazin-1-yl)sulfonyl)-1H-indole (32 mg, 32%) as a white solid: ¹H NMR (400 MHz, CDCl₃) δ 8.58 (br s, 1H), 8.15 (s, 1H), 7.60 (d, J=8.5 Hz, 1H), 7.52 (d, J=8.3 Hz, 1H), 7.39-7.35 (m, 1H), 7.04 (s, 1H), 6.91 (s, 1H), 6.89 (s, 1H), 6.70 (s, 1H), 3.34-3.26 (m, 4H), 3.20-3.13 (m, 4H); HRMS (ESI) m/z 444.0760 [(M+H)⁺ calcd for C₁₉H₁₇ClF₃N₃O₂S 444.0755].

### Example 1L

1-(5-((4-(3-Chloro-5-(trifluoromethyl)phenyl)piperazin-1-yl)sulfonyl)-1H-indol-1-yl)ethan-1-one (Compound 40), the structure of which is shown below, was synthesized according to the following procedure.

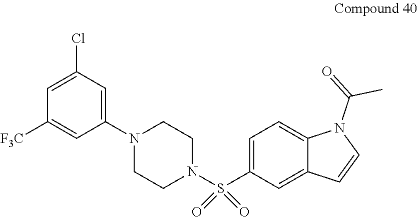 [SMILES: CC(=O)n1ccc2cc(S(=O)(=O)N3CCN(c4cc(Cl)cc(C(F)(F)F)c4)CC3)ccc21]

To a solution of 5-((4-(3-chloro-5-(trifluoromethyl)phenyl)piperazin-1-yl)sulfonyl)-1H-indole (15 mg, 0.03 mmol), Et₃N (12 μL, 0.09 mmol), and N,N-dimethyl-4-aminopyridine (1.4 mg, 0.012 mmol) in anhydrous 1,2-dichloroethane (1 mL) was added Ac₂O (11.2 μL, 0.12 mmol). The resulting mixture was stirred at 80° C. for 24 h, the reaction was quenched by an addition of H₂O. The mixture was extracted with EtOAc. The combined organic layers were dried over anhydrous Na₂SO₄ and concentrated in vacuo. The residue was purified by column chromatography (silica gel, hexanes/EtOAc, 3/1) to afford Compound 40 (14 mg, 96%) as a white solid: ¹H NMR (400 MHz, CDCl₃) δ 8.63 (d, J=8.8 Hz, 1H), 8.05 (s, 1H), 7.75 (d, J=9.1 Hz, 1H), 7.58 (d, J=3.7 Hz, 1H), 7.04 (s, 1H), 6.92 (s, 1H), 6.89 (s, 1H), 6.77 (d, J=3.7 Hz, 1H), 3.37-3.25 (m, 4H), 3.24-3.12 (m, 4H), 2.69 (s, 3H); HRMS (ESI) m/z 486.0867 [(M+H)⁺ calcd for C₂₁H₁₉ClF₃N₃O₃S 486.0872].

### Example 1M

5-((4-(3-Chloro-5-(trifluoromethyl)phenyl)piperazin-1-yl)sulfonyl)indoline, the structure of which is shown below, was synthesized according to the following procedure.

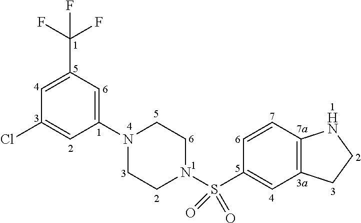 [SMILES: O=S(=O)(c1ccc2c(c1)CCN2)N1CCN(c2cc(Cl)cc(C(F)(F)F)c2)CC1]

c-HCl (0.2 mL) was added to a cooled (0° C.) solution of Compound 31 (the structure of which is shown above) (89 mg, 0.18 mmol) in EtOH (0.41 mL). The resulting mixture was refluxed for 2 h and then ice water was added followed by 35% NH₄OH. The reaction was extracted with EtOAc. The combined organic layers were dried over anhydrous Na₂SO₄ and concentrated in vacuo to afford 5-((4-(3-Chloro-5-(trifluoromethyl)phenyl)piperazin-1-yl)sulfonyl)indoline (80 mg). 5-((4-(3-Chloro-5-(trifluoromethyl)phenyl)piperazin-1-yl)sulfonyl)indoline compounds were used in the following step without further purification: ¹H NMR (400 MHz, CDCl₃) δ 7.42 (d, J=6.2 Hz, 1H), 7.41 (s, 1H), 7.03 (s, 1H), 6.93 (s, 1H), 6.90 (s, 1H), 6.57 (d, J=8.8 Hz, 1H), 3.68 (t, J=8.4 Hz, 2H), 3.32-3.25 (m, 4H), 3.15-3.09 (m, 4H), 3.08 (t, J=8.5 Hz, 2H).

### Example 1N

5-((4-(3-Chloro-5-(trifluoromethyl)phenyl)piperazin-1-yl)sulfonyl)-1-(methylsulfonyl)indoline, the structure of which is shown below, was synthesized according to the following procedure.

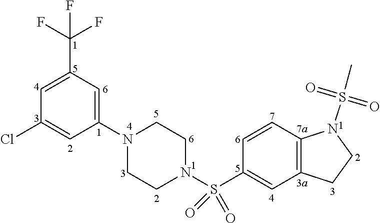 [SMILES: CS(=O)(=O)N1CCc2cc(S(=O)(=O)N3CCN(c4cc(Cl)cc(C(F)(F)F)c4)CC3)ccc21]

Methanesulfonyl chloride (0.03 mL, 0.39 mmol) was added to a solution of 5-((4-(3-Chloro-5-(trifluoromethyl)phenyl)piperazin-1-yl)sulfonyl)indoline (36 mg, 0.08 mmol) in pyridine (3.5 mL). The reaction mixture was stirred at 25° C. for 18 h. The reaction was quenched by an addition of saturated NH₄Cl solution and the organic layer was extracted with CH₂Cl₂. The combined organic layers were dried over anhydrous Na₂SO₄ and concentrated in vacuo. The residue was purified by column chromatography (silica gel, hexanes/EtOAc, 2/1) to afford 5-((4-(3-Chloro-5-(trifluoromethyl)phenyl)piperazin-1-yl)sulfonyl)-1-(methylsulfonyl)indoline as a white solid (29 mg, 67% for 2 steps): ¹H NMR (400 MHz, CDCl₃) δ 7.62 (d, J=8.5 Hz, 1H), 7.59 (s, 1H), 7.49 (d, J=7.5 Hz, 1H), 7.06 (s, 1H), 6.94 (s, 1H), 6.91 (s, 1H), 4.08 (t, J=8.6 Hz, 2H), 3.33-3.27 (m, 4H), 3.23 (t, J=8.6 Hz, 2H), 3.17-3.12 (m, 4H), 2.95 (s, 3H); HRMS (ESI) m/z 524.0689 [(M+H)⁺ calcd for C₂₀H₂₁ClF₃N₃O₄S₂ 524.0687].

### Example 1O

Methyl 4-(3-(4-((4-(3-(trifluoromethyl)phenyl)piperazin-1-yl)sulfonyl)phenyl)ureido)butanoate, the structure of which is shown below, was synthesized according to the following procedure.

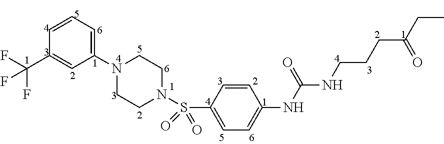 [SMILES: CCC(=O)CCCNC(=O)Nc1ccc(S(=O)(=O)N2CCN(c3cccc(C(F)(F)F)c3)CC2)cc1]

DIPEA (0.23 mL, 1.33 mmol) was added to a solution of 4-((4-(3-(trifluoromethyl)phenyl)piperazin-1-yl)sulfonyl)aniline (103 mg, 0.26 mmol) and CDI (216 mg, 1.33 mmol) in anhydrous THF (1.3 mL). After stirring at 25° Q for 2 h, methyl 4-aminobutanoate (204 mg, 1.33 mmol) was added to the reaction mixture. After stirring at 25° C. for 1.5 h, the reaction mixture was concentrated under reduced pressure. The residue was purified by column chromatography (silica gel, hexanes/EtOAc, 1/1) to afford methyl 4-(3-(4-((4-(3-(trifluoromethyl)phenyl)piperazin-1-yl)sulfonyl)phenyl)ureido)butanoate (100 mg, 71%) as a colorless oil: ¹H NMR (400 MHz, CDCl₃) δ 7.64 (d, J=8.5 Hz, 2H), 7.56 (d, J=8.7 Hz, 2H), 7.37-7.29 (m, 1H), 7.11 (d, J=7.6 Hz, 1H), 7.06 (s, 1H), 7.01 (d, J=8.6 Hz, 1H), 5.55 (br s, 1H), 3.68 (s, 2H), 3.34-3.23 (m, 4H), 3.16-3.08 (m, 4H), 2.41 (t, J=6.8 Hz, 2H), 1.91-1.83 (m, 2H); HRMS (ESI) m/z 529.1726 [(M+H)⁺ calcd for C₂₃H₂₇F₃N₄O₅S 529.1727].

### Example 1P

4-(3-(4-((4-(3-(Trifluoromethyl)phenyl)piperazin-1-yl)sulfonyl)phenyl)ureido)butanoic acid (Compound 34), the structure of which is shown below, was synthesized according to the following procedure.

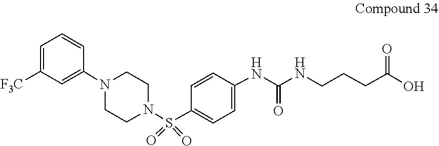 [SMILES: O=C(O)CCCNC(=O)Nc1ccc(S(=O)(=O)N2CCN(c3cccc(C(F)(F)F)c3)CC2)cc1]

1 N LiOH (0.37 mL) was added to a solution of methyl 4-(3-(4-((4-(3-(trifluoromethyl)phenyl)piperazin-1-yl)sulfonyl)phenyl)ureido)butanoate (100 mg, 0.18 mmol) in THF/H₂O (2/1, 1.8 mL) at a temperature of 25° C. After stirring for 18 h, the reaction was quenched by an addition of 1 N HCl, and the resulting mixture was diluted with CH₂Cl₂. The layers were separated, and the aqueous layer was extracted with CH₂Cl₂. The combined organic layers were dried over anhydrous Na₂SO₄ and concentrated in vacuo. The residue was purified by column chromatography (silica gel, CH₂Cl₂/MeOH (10/1) to 100% MeCN) to afford Compound 34 (90 mg, 97%) as a colorless oil: ¹H NMR (400 MHz, CD₃OD) δ 7.68 (d, J=8.8 Hz, 2H), 7.62 (d, J=8.7 Hz, 2H), 7.37 (dd, J=7.8, 7.9 Hz, 1H), 7.15 (d, J=8.6 Hz, 1H), 7.14 (s, 1H), 7.08 (d, J=7.6 Hz, 1H), 3.28-3.22 (m, 6H), 3.14-3.09 (m, 4H), 2.35 (t, J=7.3 Hz, 2H), 1.84-1.82 (m, 2H); HRMS (ESI) m/z 515.1570 [(M+H)⁺ calcd for C₂₂H₂₅F₃N₄O₅S 515.1571].

### Example 1Q

N-((tert-Butyldimethylsilyl)oxy)-4-(3-(4-((4-(3-(trifluoromethyl)phenyl)piperazin-1-yl)sulfonyl)phenyl)ureido)butanamide, the structure of which is shown below, was synthesized according to the following procedure.

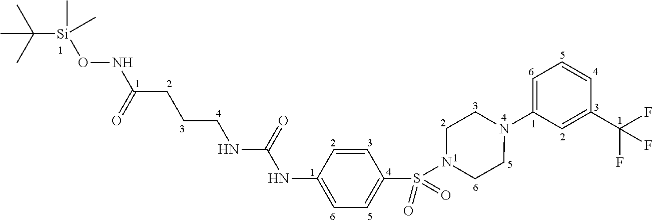 [SMILES: CC(C)(C)[Si](C)(C)ONC(=O)CCCNC(=O)Nc1ccc(S(=O)(=O)N2CCN(c3cccc(C(F)(F)F)c3)CC2)cc1]

To a solution of 4-(3-(4-((4-(3-(Trifluoromethyl)phenyl)piperazin-1-yl)sulfonyl)phenyl)ureido)butanoic acid (Compound 34) (22 mg, 0.04 mmol) in anhydrous THF (2.86 mL), ethyl chloroformate (8 μL, 0.08 mmol) and Et₃N (11 μL, 0.08 mmol) were added. After stirring at 25° C. for 1 h, NH₂OTBS (12.5 mg, 0.08 mmol) in anhydrous MeOH (0.66 mL) was added to the reaction mixture. After stirring at 25° C. for 1 h, the reaction mixture was concentrated in vacuo. The residue was purified by column chromatography (silica gel, CH₂Cl₂/MeOH, 50/1 to 10/1) to afford N-((tert-Butyldimethylsilyl)oxy)-4-(3-(4-((4-(3-(trifluoromethyl)phenyl)piperazin-1-yl)sulfonyl)phenyl)ureido)butanamid (23 mg, 85%) as a white solid: ¹H NMR (400 MHz, CD₃OD) δ 7.68 (d, J=9.3 Hz, 2H), 7.62 (d, J=9.1 Hz, 2H), 7.37 (dd, J=7.9, 8.0 Hz, 1H), 7.14 (d, J=7.6 Hz, 1H), 7.13 (s, 1H), 7.08 (d, J=7.7 Hz, 1H), 6.35-6.30 (m, 1H), 3.32-3.21 (m, 6H), 3.13-3.09 (m, 4H), 2.17 (t, J=7.4 Hz, 2H), 1.87-1.78 (m, 2H), 0.95 (s, 9H), 0.16 (s, 6H); HRMS (ESI) m/z 644.2542 [(M+H)⁺ calcd for C₂₈H₄₀F₃N₅O₅SSi 644.2544].

### Example 1R

N-Hydroxy-4-(3-(4-((4-(3-(trifluoromethyl)phenyl)piperazin-1-yl)sulfonyl)phenyl)ureido)butanamide (Compound 36), the structure of which is shown below, was synthesized according to the following procedure.

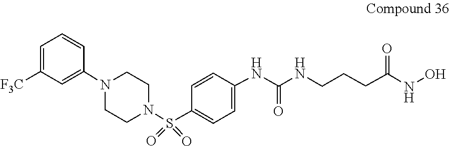 [SMILES: O=C(CCCNC(=O)Nc1ccc(S(=O)(=O)N2CCN(c3cccc(C(F)(F)F)c3)CC2)cc1)NO]

TFA (0.5 mL) was added dropwise to a cooled (0° C.) solution of N-((tert-Butyldimethylsilyl)oxy)-4-(3-(4-((4-(3-(trifluoromethyl)phenyl)piperazin-1-yl)sulfonyl)phenyl)ureido)butanamide (7.7 mg, 0.01 mmol) in anhydrous CH₂Cl₂ (1.7 mL). After stirring at 25° C. for 25 min, the reaction mixture was concentrated in vacuo. The residue was purified by column chromatography (silica gel, CH₂Cl₂/MeOH, 10/1) to afford Compound 36 as a white solid (7 mg, quantitative): ¹H NMR (400 MHz, CD₃OD) δ 7.68 (d, J=8.7 Hz, 2H), 7.62 (d, J=8.4 Hz, 2H), 7.38 (dd, J=7.8, 7.8 Hz, 1H), 7.16 (d, J=8.1 Hz, 1H), 7.14 (s, 1H), 7.09 (d, J=7.8 Hz, 1H), 3.29-3.19 (m, 6H), 3.15-3.07 (m, 4H), 2.15 (t, J=7.5 Hz, 2H), 1.87-1.78 (m, 2H); HRMS (ESI) m/z 530.1680 [(M+H)⁺ calcd for C₂₂H₂₆F₃N₅O₅S 530.1680].

### Example 1S

Methyl 5-(3-(4-((4-(3-(trifluoromethyl)phenyl)piperazin-1-yl)sulfonyl)phenyl)ureido)pentanoate, the structure of which is shown below, was synthesized according to the following procedure.

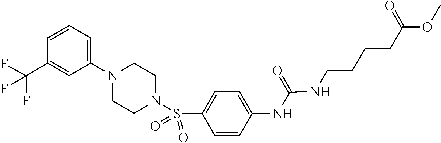 [SMILES: COC(=O)CCCCNC(=O)Nc1ccc(S(=O)(=O)N2CCN(c3cccc(C(F)(F)F)c3)CC2)cc1]

DIPEA was added (0.2 mL, 1.14 mmol) to a solution of 4-((4-(3-(trifluoromethyl)phenyl)piperazin-1-yl)sulfonyl)aniline (88 mg, 0.23 mmol) and CDI (185 mg, 1.14 mmol) in THF (1.14 mL). After stirring at 25° C. for 2 h, the reaction mixture was treated with methyl 5-aminopentanoate (150 mg, 1.14 mmol) and allowed to stir for 1 h. The reaction mixture was concentrated in vacuo and the residue was purified by column chromatography (silica gel, hexanes/EtOAc, 1/1) to afford methyl 5-(3-(4-((4-(3-(trifluoromethyl)phenyl)piperazin-1-yl)sulfonyl)phenyl)ureido)pentanoate (75 mg, 60%) as a colorless oil: ¹H NMR (400 MHz, CDCl₃) δ 7.73 (s, 1H), 7.62 (d, J=8.9 Hz, 2H), 7.53 (d, J=8.7 Hz, 2H), 7.33 (dd, J=8.2, 8.2 Hz, 1H), 7.10 (d, J=7.7 Hz, 1H), 7.05 (s, 1H), 7.00 (d, J=8.2 Hz, 1H), 5.60 (t, J=5.5 Hz, 1H), 3.64 (s, 3H), 3.27-3.22 (m, 6H), 3.13-3.11 (m, 4H), 2.33 (t, J=7.1 Hz, 2H), 1.70-1.62 (m, 2H), 1.58-1.51 (m, 2H).

### Example 1T

5-(3-(4-((4-(3-(Trifluoromethyl)phenyl)piperazin-1-yl)sulfonyl)phenyl)ureido)pentanoic acid (Compound 35), the structure of which is shown below, was synthesized according to the following procedure.

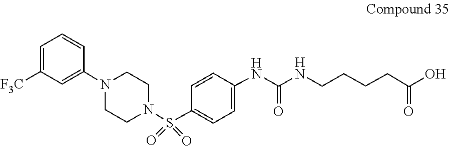 [SMILES: O=C(O)CCCCNC(=O)Nc1ccc(S(=O)(=O)N2CCN(c3cccc(C(F)(F)F)c3)CC2)cc1]

1 N LiOH (0.25 mL) was added to methyl 5-(3-(4-((4-(3-(trifluoromethyl)phenyl)piperazin-1-yl)sulfonyl)phenyl)ureido)pentanoate (34 mg, 0.063 mmol) in THF/H₂O (2/1, 0.63 mL) at 25° C. After stirring at 25° C. for 17 h, the reaction mixture was cooled to 0° C. and acidified with 1 N HCl. After an addition of H₂O, the resulting mixture was extracted with CH₂Cl₂. The organic layers were combined, dried over anhydrous Na₂SO₄, and concentrated in vacuo. The residue was purified by column chromatography (silica gel, CH₂Cl₂/MeOH, 10/1) to afford Compound 35 (18 mg, 54%) as a white solid: ¹H NMR (400 MHz, CD₃OD) δ 7.67 (d, J=9.0 Hz, 2H), 7.62 (d, J=9.0 Hz, 2H), 7.37 (dd, J=8.3, 8.3 Hz, 1H), 7.15 (d, J=7.4 Hz, 1H), 7.14 (s, 1H), 7.08 (d, J=7.6 Hz, 1H), 3.29-3.26 (m, 4H), 3.22 (t, J=6.7 Hz, 2H), 3.11-3.10 (m, 4H), 2.34 (t, J=8.0 Hz, 2H), 1.70-1.63 (m, 2H), 1.60-1.53 (m, 2H); HRMS (ESI) m/z 529.1733 [(M+H)⁺ calcd for C₂₃H₂₇F₃N₄O₅S 529.1727].

### Example 1U

N-Hydroxy-5-(3-(4-((4-(3-(trifluoromethyl)phenyl)piperazin-1-yl)sulfonyl)phenyl)ureido)pentanamide (Compound 37), the structure of which is shown below, was synthesized according to the following procedure.

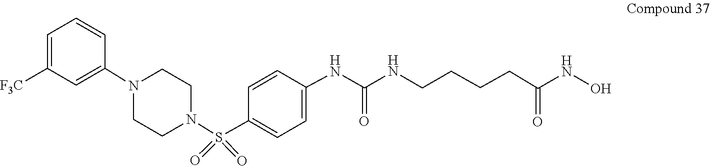 [SMILES: O=C(CCCCNC(=O)Nc1ccc(S(=O)(=O)N2CCN(c3cccc(C(F)(F)F)c3)CC2)cc1)NO]

Ethyl chloroformate (6.2 mg, 0.057 mmol) and Et₃N (8 μL) were added to a solution of 5-(3-(4-((4-(3-(trifluoromethyl)phenyl)piperazin-1-yl)sulfonyl)phenyl)ureido)pentanoic acid (Compound 35) (15 mg, 0.028 mmol) in THF (2 mL). After stirring for 1 h, the reaction mixture was treated with NH₂OTBS (8.4 mg, 0.057 mmol) in MeOH (0.44 mL). After an additional 2 h, NH₂OTBS (8.4 mg, 0.057 mmol) in MeOH (0.44 mL) was added and the resulting mixture was left to stir for additional 2 h. The reaction mixture was concentrated in vacuo, dissolved in CH₂Cl₂ (2 mL), and treated with TFA (0.17 mL). The resulting mixture was stirred at 0° C. for 1 h and then concentrated in vacuo to afford Compound 37 (8.1 mg, 53% for 2 steps) as a solid: ¹H NMR (400 MHz, CD₃OD) δ 7.69 (d, J=8.9 Hz, 2H), 7.62 (d, J=8.9 Hz, 2H), 7.39 (dd, J=7.9, 7.9 Hz, 1H), 7.19 (d, J=8.3 Hz, 1H), 7.18 (s, 1H), 7.11 (d, J=7.5 Hz, 1H), 3.32-3.30 (m, 4H), 3.22 (t, J=6.7 Hz, 2H), 3.14-3.11 (m, 4H), 2.14 (t, J=7.3 Hz, 2H), 1.72-1.63 (m, 2H), 1.61-1.50 (m, 2H); HRMS (ESI) m/z 544.1840 [(M+H)⁺ calcd for C₂₃H₂₈F₃N₅O₅S 544.1836].

### Example 1V

Methyl 5-(3-(4-((4-(3-chloro-5-(trifluoromethyl)phenyl)piperazin-1-yl)sulfonyl)phenyl)ureido)pentanoate, the structure of which is shown below, was synthesized according to the following procedure.

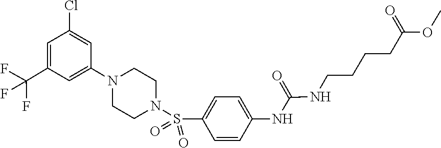 [SMILES: COC(=O)CCCCNC(=O)Nc1ccc(S(=O)(=O)N2CCN(c3cc(Cl)cc(C(F)(F)F)c3)CC2)cc1]

DIPEA (0.6 mL, 3.46 mmol) was added to a solution of 4-((4-(3-(trifluoromethyl)phenyl)piperazin-1-yl)sulfonyl)aniline (290 mg, 0.69 mmol) and CDI (561 mg, 3.46 mmol) in anhydrous THF (3.45 mL). After stirring at 25° C. for 2 h, the reaction mixture was transferred to methyl 5-aminopentanoate (114 mg, 0.87 mmol). After stirring at 25° C. for 1.5 h, the reaction mixture was concentrated under reduced pressure and the residue was purified by column chromatography (silica gel, hexanes/EtOAc, 1/1) to afford methyl 5-(3-(4-((4-(3-chloro-5-(trifluoromethyl)phenyl)piperazin-1-yl)sulfonyl)phenyl)ureido)pentanoate (350 mg, 87%) as a white sticky solid: ¹H NMR (400 MHz, CDCl₃) δ 7.63 (s, 1H), 7.59 (d, J=8.5 Hz, 2H), 7.49 (d, J=8.2 Hz, 2H), 7.05 (s, 1H), 6.95 (s, 1H), 6.93 (s, 1H), 5.56 (br s, 1H), 3.65 (s, 3H), 3.34-3.20 (m, 6H), 3.15-3.06 (m, 4H), 2.33 (t, J=7.2 Hz, 2H), 1.69-1.59 (m, 2H), 1.58-1.50 (m, 2H).

### Example 1W

5-(3-(4-((4-(3-Chloro-5-(trifluoromethyl)phenyl)piperazin-1-yl)sulfonyl)phenyl)ureido)pentanoic acid (Compound 38), the structure of which is shown below, was synthesized according to the following procedure.

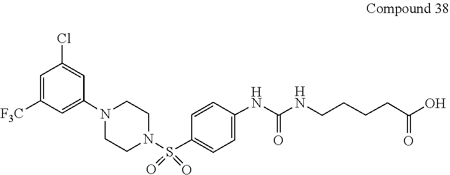 [SMILES: O=C(O)CCCCNC(=O)Nc1ccc(S(=O)(=O)N2CCN(c3cc(Cl)cc(C(F)(F)F)c3)CC2)cc1]

To a solution of methyl 5-(3-(4-((4-(3-chloro-5-(trifluoromethyl)phenyl)piperazin-1-yl)sulfonyl)phenyl)ureido)pentanoate (34 mg, 0.05 mmol) in THF/H₂O (2/1, 0.5 mL), 1 N LIOH (0.13 mL) was added at a temperature of 25° C. After stirring for 18 h, the reaction was quenched by an addition of 1 N HCl, and the resulting mixture was diluted with CH₂Cl₂. The layers were separated, and the aqueous layer was extracted with CH₂Cl₂. The combined organic layers were dried over anhydrous Na₂SO₄ and concentrated in vacuo. The residue was purified by column chromatography (silica gel, CH₂Cl₂/MeOH (30/1) to 100% MeCN) to afford Compound 38 (15 mg, 45%) as a white solid: ¹H NMR (400 MHz, CD₃COCD₃) δ 8.60 (s, 1H), 7.75 (d, J=8.6 Hz, 2H), 7.65 (d, J=8.6 Hz, 2H), 7.20 (s, 1H), 7.17 (s, 1H), 7.06 (s, 1H), 6.18 (br s, 1H), 3.50-3.41 (m, 4H), 3.27-3.24 (m, 2H), 3.15-3.07 (m, 4H), 2.33 (t, J=7.2 Hz, 2H), 1.67-1.64 (m, 2H), 1.59-1.56 (m, 2H); HRMS (ESI) m/z 563.1340 [(M+H)⁺ calcd for C₂₃H₂₆ClF₃N₄O₅S 563.1337].

### Example 1X

N-Hydroxy-5-(3-(4-((4-(3-chloro-5-(trifluoromethyl)phenyl)piperazin-1-yl)sulfonyl)phenyl)ureido)-pentanamide (Compound 39), the structure of which is shown below, was synthesized via a coupling reaction and TBS deprotection using the following procedure.

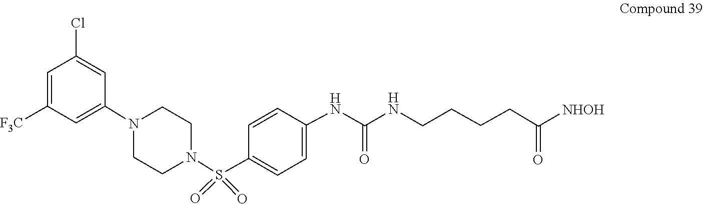

Ethyl chloroformate (4.3 μL, 0.04 mmol) and Et₃N (6 μL, 0.04 mmol) were added to a solution of 5-(3-(4-((4-(3-chloro-5-(trifluoromethyl)phenyl)piperazin-1-yl)sulfonyl)phenyl)ureido)pentanoic acid (Compound 38) (13 mg, 0.02 mmol) in anhydrous THF (1.69 mL). After stirring at 25° C. for 1 h, NH₂OTBS (6 mg, 0.04 mmol) in anhydrous MeOH (0.39 mL) was added to the reaction mixture. After stirring at 25° C. for 1 h, the reaction mixture was concentrated in vacuo and purified by column chromatography (silica gel, CH₂Cl₂/MeOH, 30/1) to afford N-((tert-butyldimethylsilyl)oxy)-5-(3-(4-((4-(3-chloro-5-(trifluoromethyl)phenyl)piperazin-1-yl)sulfonyl)phenyl)ureido)pentanamide (13 mg) as a white solid: ¹H NMR (400 MHz, CD₃COCD₃) δ 9.78 (br s, 1H), 8.46 (s, 1H), 7.74 (d, J=9.2 Hz, 2H), 7.66 (d, J=8.9 Hz, 2H), 7.20 (s, 1H), 7.17 (s, 1H), 7.06 (s, 1H), 6.04 (t, J=5.5 Hz, 1H), 3.50-3.42 (m, 4H), 3.26-3.19 (m, 2H), 3.15-3.07 (m, 4H), 2.15-2.13 (m, 2H), 1.69-1.60 (m, 2H), 1.56-1.53 (m, 2H), 0.94 (s, 9H), 0.15 (s, 6H); HRMS (ESI) m/z 692.2313 [(M+H)⁺ calcd for C₂₉H₄₁ClF₃N₅O₅SSi 692.2311]. To achieve TBS deprotection, TFA (240 μL) was added dropwise to a cooled (0° C.) solution of the TBS protected hydroxamic acid (13 mg, 0.01 mmol) in anhydrous CH₂Cl₂ (2.8 mL). After stirring for 25 min at 25° C., the reaction mixture was concentrated in vacuo and purified by column chromatography (silica gel, CH₂Cl₂/MeOH, 10/1) to afford (Compound 39) (7 mg, 55% for 2 steps) as a white solid: ¹H NMR (400 MHz, CD₃COCD₃) δ 10.04 (br s, 1H), 8.09 (br s, 1H), 7.74 (br s, 2H), 7.66 (br s, 2H), 7.21 (s, 1H), 7.18 (s, 1H), 7.06 (s, 1H), 3.49-3.37 (m, 4H), 3.26-3.18 (m, 2H), 3.12-3.10 (m, 4H), 2.19-2.11 (m, 2H), 1.70-1.59 (m, 2H), 1.56-1.48 (m, 2H); HRMS (ESI) m/z 578.1441 [(M+H)⁺ calcd for C₂₃H₂₇ClF₃N₅O₅S 578.1446].

### Example 2: Cloning and Purification of K. pneumoniae LpxH

K. pneumoniae LpxH was cloned and purified for crystallography analysis. Briefly, K. pneumoniae LpxH was cloned into a modified pET21 b (Novagen/Millipore Sigma) vector, yielding the LpxH fusion protein with a C-terminal TEV protease site (ENLYFQGS) and His₁₀ tag. Vector-transformed BL21 STAR (DE3) E. coli cells (Thermo Fisher Scientific) were grown in M9 minimal medium to an OD₆₀₀ of 0.5 at 37° C., prior to being induced with 1 mM IPTG at 30° C. After 5 h, the cells were then harvested by centrifugation. Protein purification was carried out at 4° C. Cell pellets were lysed in a buffer containing 50 mM phosphate-citrate, 20 mM MES (pH 6.0), 600 mM NaCl, 10% sucrose, 5 mM 2-mercaptoethanol, 10 mM imidazole, and 0.1% Triton X-100 using a French press. After removing cell debris by centrifugation, a HisPur Ni-NTA column (Thermo Fisher Scientific) was pre-equilibrated with the lysis buffer, and the supernatant was loaded. Following extensive washes using a purification buffer containing 20 mM phosphate-citrate, 20 mM MES (pH 6.0), 300 mM NaCl, 5% glycerol, 5 mM 2-mercaptoethanol, and 40 mM imidazole, LpxH was eluted from the column by increasing the imidazole concentration stepwise from 40 to 400 mM. The protein sample was concentrated and further purified with size-exclusion chromatography (Superdex 200 chromatography available from GE Healthcare Life Sciences) in the FPLC buffer containing 20 mM MES (pH 6.0), 800 mM NaCl, 1 mM DTT, and 5% glycerol.

### Example 3: Co-Crystallization of K. pneumoniae LpxH with N-Hydroxy-5-(3-(4-((4-(3-chloro-5-(trifluoromethyl)phenyl)piperazin-1-yl)sulfonyl)phenyl)ureido)-pentanamide (Compound 39)

Peak fractions containing K. pneumoniae LpxH were buffer-exchanged into a buffer containing 20 mM MES (pH 6.0), 200 mM NaCl, 1 mM DTT, and 5% glycerol. During buffer exchange, concentrated N-Hydroxy-5-(3-(4-((4-(3-chloro-5-(trifluoromethyl)phenyl)piperazin-1-yl)sulfonyl)phenyl)ureido)-pentanamide (Compound 39) solution in DMSO was added to the protein solution in a 1:1 molar ratio. The solution was then concentrated to 8 mg/mL for crystallization and additional concentrated Compound 39 solution in DMSO was added to the protein solution in a 1:1 molar ratio (final ratio=2:1 drug: protein, final Compound 39 concentration=0.54 mM, DMSO=2%).

Protein crystals were then grown using the sitting-drop vapor diffusion method at 20° C. Each drop was prepared by mixing 1 μL of the protein solution with 1 μL of the reservoir solution (200 mM KCl, 100 mM sodium citrate, 37% pentaerythritol propoxylate (5/4 PO/OH), pH 5.5). The final drop solution contained 4 mg/mL of LpxH with 0.27 mM Compound 39, 10 mM MES (pH 6.0), 100 mM NaCl, 100 mM KCl, 50 mM sodium citrate (pH 5.5), 18.5% pentaerythritol propoxylate (5/4 PO/OH), 0.5 mM DTT, 1% DMSO, and 2.5% glycerol. Diffraction quality protein crystals were harvested after 2 weeks and soaked with the reservoir solution additionally containing 20% glycerol, 100 μM MnCl₂, 0.27 mM Compound 39, and 2% DMSO for cryoprotection.

### Example 4: Structural Analysis of K. pneumoniae LpxH Complexes with N-Hydroxy-5-(3-(4-((4-(3-chloro-5-(trifluoromethyl)phenyl)piperazin-1-yl)sulfonyl)phenyl)ureido)-pentanamide (Compound 39)

The X-ray diffraction data of the K. pneumoniae LpxH complex with N-Hydroxy-5-(3-(4-((4-(3-chloro-5-(trifluoromethyl)phenyl)piperazin-1-yl)sulfonyl)phenyl)ureido)-pentanamide (Compound 39) were collected at the Northeastern Collaborative Access Team (NECAT) 24-ID-C beamline at the Advanced Photon Source at Argonne National Laboratory. The X-ray diffraction data was processed using X-ray Detector Software, namely XDS Pagram Package Version of Jan. 31, 2020. The phase information of the crystal structures of the K. pneumoniae LpxH complex was obtained by molecular replacement with the PHASER module in the Python-based Hierarchical ENvironment for Integrated Xtallography (PHENIX) suite using the Protein Data Bank (PDB) entry 6PJ3 as the search model. Restraints of the inhibitors were generated by using Ligand Builder and Optimization Workbench (eLBOW), and edited manually. Discussion of eLBOW may be found in Moriarty, N. W., Grosse-Kunstleve, R. W., Adams, P. D. (2009) electronic Ligand Builder and Optimization Workbench (eLBOW): a tool for ligand coordinate and restraint generation. Acta Crystallogr. D Biol. Crystallogr. 65 (10), 1074-1080. https://doi.org/10.1107/S0907444909029436, which is incorporated herein in its entirety for all purposes. Iterative model building and refinement was carried out using COOT, which is an open-source model-building program written by Paul Emsley, and PHENIX. The 2mFo-DFc omit maps were generated using PHENIX. Discussion of COOT may be found in Emsley, P., Cowtan, K. (2004) Coot: model-building tools for molecular graphics. Acta Crystallogr. D Biol. Crystallogr. 60 (12), 2126-2132. https://doi.org/10.1107/S0907444904019158, which is incorporated herein in its entirety for all purposes.

### Example 5: Enzymatic Assay for LpxH Inhibition

A LpxE-coupled LpxH activity assay was conducted. Briefly, two reaction mixtures were prepared that contain 20 mM Tris-HCl (pH 8.0), 0.5 mg/mL BSA, 0.02% Triton X-100, 1 mM MnCl₂, 1 mM DTT, and 10% DMSO, with one additionally containing 200 μM substrate (UDP-DAGn) and the other containing both LpxH (20 ng/mL) and 0.2 μM inhibitor. The reaction mixtures were pre-incubated at 37° C. for 10 minutes before an equal volume of the LpxH mixture was added to the substrate mixture to initiate the reaction at 37° C. The final reaction solution contains 100 μM substrate, 10 ng/mL enzyme, and 0.1 μM inhibitor. At the desired reaction time points, an aliquot of 20 μL reaction mixture was removed and added to a well in 96-well half-area plate containing 5 mM EDTA (final concentration) to quench the LpxH reaction. Purified Aquifex aeolicus LpxE was then added to a final concentration of 5 μg/mL. The plate was incubated at 37° C. for 30 minutes followed by addition of formic acid to a final concentration of 3.75 M to quench the LpxE reaction. The malachite green reagent (available from SIGMA ALDRICH, catalog MAK307) was added with a 5-fold dilution, and the solution was incubated for 30 minutes at room temperature before the absorbance at 620 nm was measured. All measurements were done in triplicates, and standard error was calculated. Percentage LpxH activities for Compound 1, Compound 26, and Compound 31, the structures of which are disclosed herein, at 0.1 μM were calculated from previously reported IC₅₀ values,¹³ which were extracted from fitting of the dose-response curve of vᵢ/vₒ=1/(1+[I]/IC₅₀) assayed under identical conditions.

Further discussion regarding LpxH activity assays is discussed in Cho, J., Lee, M., Cochrane, C. S., Webster, C. G., Fenton, B. A., Zhao, J., Hong, J., Zhou, P. (2020) Structural basis of the UDP-diacylglucosamine pyrophosphohydrolase LpxH inhibition by sulfonyl piperazine antibiotics. Proc. Natl. Acad. Sci. U.S.A. 117 (8) 4109-4116, https://doi.org/10.1073/pnas.1912876117 and Lee, M., Zhao, J., Kwak, S. H., Cho, J., Lee, M., Gillespie, R. A., Kwon, D. Y., Lee, H., Park, H. J., Wu, Q., Zhou, P., Hong, J. (2019) Structure-activity relationship of sulfonyl piperazine LpxH inhibitors analyzed by an LpxE-coupled malachite green assay, ACS Infect. Dis. 5 (4), 641-651, https://doi.org/10.1021/acsinfecdis.8b00364, which are incorporated herein in its entirety for all purposes.

### Example 6: Synthesis Analogs with Aryl Group Modifications

Aryl group analogs were prepared by replacing the m-chloro group of Compound 31 with various functional groups, including Br, CH₃, and CF₃, e.g. according to Reaction Scheme 1, provided below, to evaluate the importance and to evaluate the effect of the size at m-position of the phenyl ring of 3. The following analog compounds were prepared: m-bromo analog 1-(5-((4-(3-bromo-5-(trifluoromethyl)phenyl)piperazin-1-yl)sulfonyl)indolin-1-yl)ethan-1-one, m-methyl analog 1-(5-((4-(3-methyl-5-(trifluoromethyl)phenyl)piperazin-1-yl)sulfonyl)indolin-1-yl)ethan-1-one, and m-trifluoromethyl analog 1-(5-((4-(3,5-bis(trifluoromethyl)phenyl)piperazin-1-yl)sulfonyl)indolin-1-yl)ethan-1-one (see e.g., Reaction Scheme 1A).

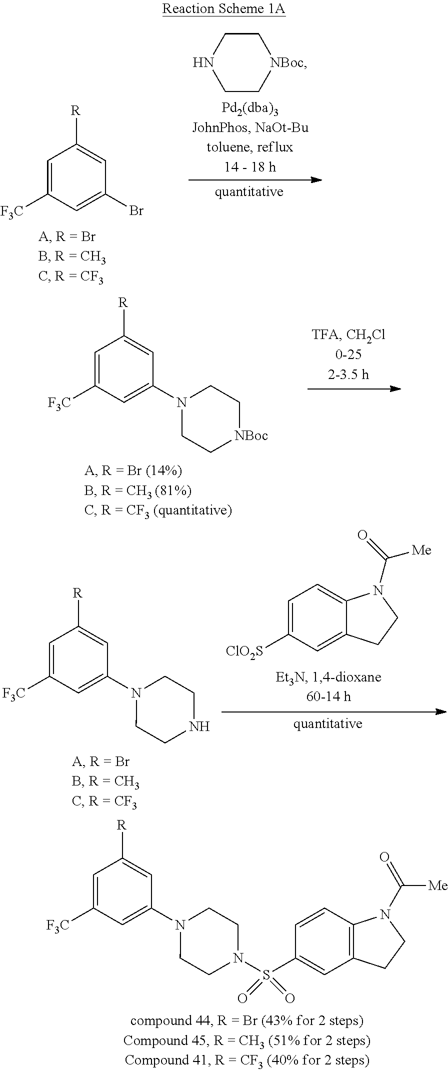

Starting from commercially available 1,3-dibromo-5-(trifluoromethyl)benzene, Boc-protected N-aryl piperazine tert-butyl 4-(3-bromo-5-(trifluoromethyl)phenyl)piperazine-1-carboxylate was produced via Pd-mediated coupling of 1,3-dibromo-5-(trifluoromethyl)benzene with 1-Boc-piperazine. Boc deprotection of tert-Butyl 4-(3-bromo-5-(trifluoromethyl)phenyl)piperazine-1-carboxylate by treatment with TFA followed by coupling of the resulting piperazine 1-(3-Bromo-5-(trifluoromethyl)phenyl)piperazine with commercially available 1-acetyl-5-indolinesulfonyl chloride in the presence of Et₃N proceeded smoothly to afford the desired m-bromo analog 1-(5-((4-(3-Bromo-5-(trifluoromethyl)phenyl)piperazin-1-yl)sulfonyl)indolin-1-yl)ethan-1-one in 43% for 2 steps. The m-methyl analog 1-(5-((4-(3-methyl-5-(trifluoromethyl)phenyl)piperazin-1-yl)sulfonyl)indolin-1-yl)ethan-1-one and the m-trifluoromethyl analog 1-(5-((4-(3,5-Bis(trifluoromethyl)phenyl)piperazin-1-yl)sulfonyl)indolin-1-yl)ethan-1-one (Compound 41) were also prepared in a similar manner starting from the commercially available 1-bromo-3-methyl-5-(trifluoromethyl)benzene and 1,3-bis(trifluoromethyl)-5-bromobenzene, respectively.

To evaluate the effect of symmetrical substituents, the m-difluoro analog 1-(5-((4-(3,5-Difluorophenyl)piperazin-1-yl)sulfonyl)indolin-1-yl)ethan-1-one and the m-dichloro analog 1-(5-((4-(3,5-Dichlorophenyl)piperazin-1-yl)sulfonyl)indolin-1-yl)ethan-1-one (Compound 42) were prepared according to Reaction Scheme 1B. Commercially available 1-bromo-3,5-difluorobenzene was coupled to 1-Boc-piperazine to afford the N-aryl piperazine tert-Butyl 4-(3,5-difluorophenyl)piperazine-1-carboxylate. Boc deprotection of tert-Butyl 4-(3,5-difluorophenyl)piperazine-1-carboxylate followed by coupling of the resulting 1-(3,5-Difluorophenyl)piperazine to 1-acetyl-5-indolinesulfonyl chloride completed the synthesis of the m-difluoro analog 1-(5-((4-(3,5-Difluorophenyl)piperazin-1-yl)sulfonyl)indolin-1-yl)ethan-1-one. Similarly, the m-dichloro analog Compound 42 was prepared by coupling commercially available 1-(3-5-dichlorophenyl)piperazine with -acetylindoline-5-sulfonyl chloride.

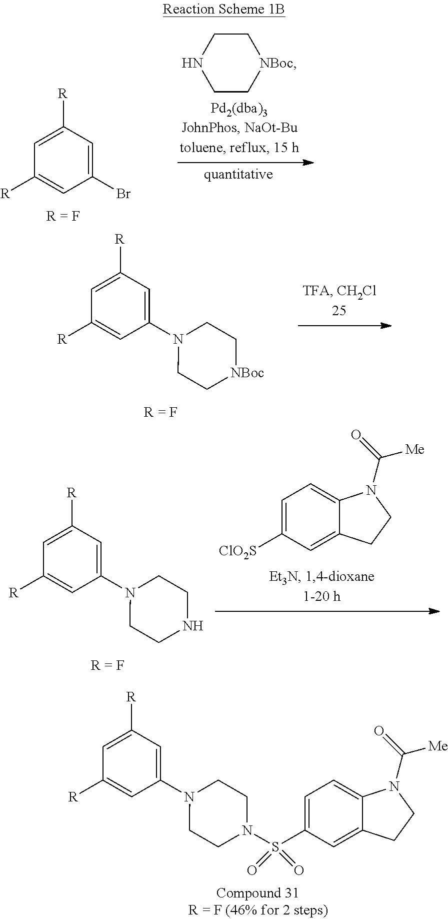

### Example 7: Synthesis Analogs with Aryl Group Modifications

Indole analog, such as 1-(5-((4-(3-Chloro-5-(trifluoromethyl)phenyl)piperazin-1-yl)sulfonyl)-1H-indol-1-yl)ethan-1-one (Compound 40), and an N-methanesulfonyl group analogs, such as 5-((4-(3-Chloro-5-(trifluoromethyl)phenyl)piperazin-1-yl)sulfonyl)-1-(methylsulfonyl)indoline, were synthesized by replacing the indoline and N-acetyl group of Compound 31, the structure of which is shown below, with an indole group and a methanesulfonyl group, respectively.

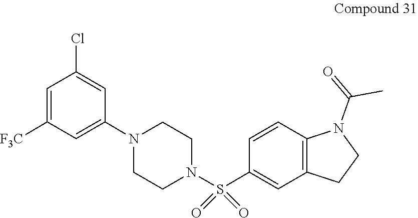 [SMILES: CC(=O)N1CCc2cc(S(=O)(=O)N3CCN(c4cc(Cl)cc(C(F)(F)F)c4)CC3)ccc21]

1-(3-Chloro-5-(trifluoromethyl)phenyl)piperazine was coupled to 1H-indole-5-sulfonyl chloride to give 5-((4-(3-Chloro-5-(trifluoromethyl)phenyl)piperazin-1-yl)sulfonyl)-1H-indole (see Reaction Scheme 2A). N-Acetylation of the indole ring of 5-((4-(3-Chloro-5-(trifluoromethyl)phenyl)piperazin-1-yl)sulfonyl)-1H-indole by treatment with Ac₂O completed the synthesis of the indole analog Compound 40. Compound Compound 31 was converted to the N-methanesulfonyl group analog 5-((4-(3-Chloro-5-(trifluoromethyl)phenyl)piperazin-1-yl)sulfonyl)-1-(methylsulfonyl)indoline by Ac deprotection under acidic conditions followed by N-methanesulfonylation of the resulting indoline.

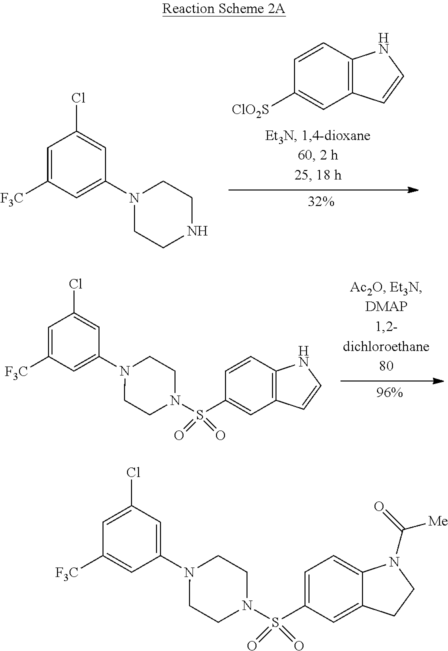 [SMILES: FC(F)(F)c1cc(Cl)cc(N2CCNCC2)c1 ; O=S(=O)(Cl)c1ccc2[nH]ccc2c1 ; O=S(=O)(c1ccc2[nH]ccc2c1)N1CCN(c2cc(Cl)cc(C(F)(F)F)c2)CC1 ; CC(=O)N1CCc2cc(S(=O)(=O)N3CCN(c4cc(Cl)cc(C(F)(F)F)c4)CC3)ccc21]

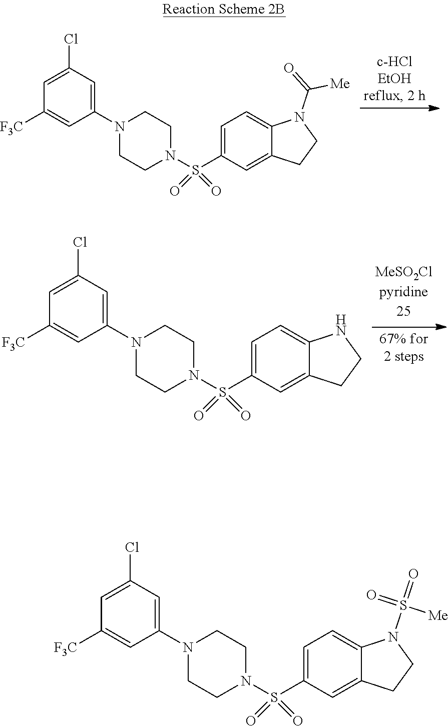 [SMILES: CC(=O)N1CCc2cc(S(=O)(=O)N3CCN(c4cc(Cl)cc(C(F)(F)F)c4)CC3)ccc21 ; O=S(=O)(c1ccc2c(c1)CCN2)N1CCN(c2cc(Cl)cc(C(F)(F)F)c2)CC1 ; CS(=O)(=O)N1CCc2cc(S(=O)(=O)N3CCN(c4cc(Cl)cc(C(F)(F)F)c4)CC3)ccc21]

### Example 8: Synthesis Analogs with Extended N-Acyl Chains

Several sulfonyl piperazine compounds with extended N-acyl chains were synthesized to explore the role of the N-acetyl group of Compound 31 in LpxH inhibition. Since LpxH contains two manganese metals in the active site, it is believed that a sulfonyl piperazine LpxH inhibitor that exploits the chelation to the di-manganese metal cluster is more potent than Compound 1 or Compound 31. Therefore, the N-acyl group was capped with a hydroxamic acid since hydroxamic acid is a well-characterized manganese-chelating group. It was believed the hydroxamic acid group would tightly bind to the manganese metals in the LpxH active site and improve the binding affinity of LpxH inhibitors. Since the N-acetylsulfanilyl analog of Compound 1 was active in our previous study, the extended N-acyl chain analogs based on a sulfanilamide scaffold. Starting from the 4-((4-(3-(trifluoromethyl)phenyl)piperazin-1-yl)sulfonyl)aniline a urea linkage with methyl 4-aminobutanoate was introduced in the presence of CDI (see, e.g., Reaction Scheme 3A). To install a hydroxamic acid group, the methyl ester methyl 4-(3-(4-((4-(3-(trifluoromethyl)phenyl)piperazin-1-yl)sulfonyl)phenyl)ureido)butanoate to the corresponding carboxylic acid 4-(3-(4-((4-(3-(Trifluoromethyl)phenyl)piperazin-1-yl)sulfonyl)phenyl)ureido)butanoic acid (Compound 34) was hydrolyzed and then coupled to Compound 34 with NH₂OTBS. The final TBS deprotection of N-((tert-Butyldimethylsilyl)oxy)-4-(3-(4-((4-(3-(trifluoromethyl)phenyl)piperazin-1-yl)sulfonyl)phenyl)ureido)butanamide by TFA successfully afforded the extended N-acyl chain hydroxamic acid analog N-Hydroxy-4-(3-(4-((4-(3-(trifluoromethyl)phenyl)piperazin-1-yl)sulfonyl)phenyl)ureido)butanamide (Compound 36). To evaluate the effect of chain length on LpxH inhibition, the acyl chain was further elongated by replacing methyl 4-aminobutanoate with methyl 5-aminopentanoate and prepared the analogs N-Hydroxy-5-(3-(4-((4-(3-(trifluoromethyl)phenyl)piperazin-1-yl)sulfonyl)phenyl)ureido)pentanamide (Compound 37) and N-Hydroxy-5-(3-(4-((4-(3-chloro-5-(trifluoromethyl)phenyl)piperazin-1-yl)sulfonyl)phenyl)ureido)-pentanamide (Compound 39) (see e.g., Reaction Scheme 3B).

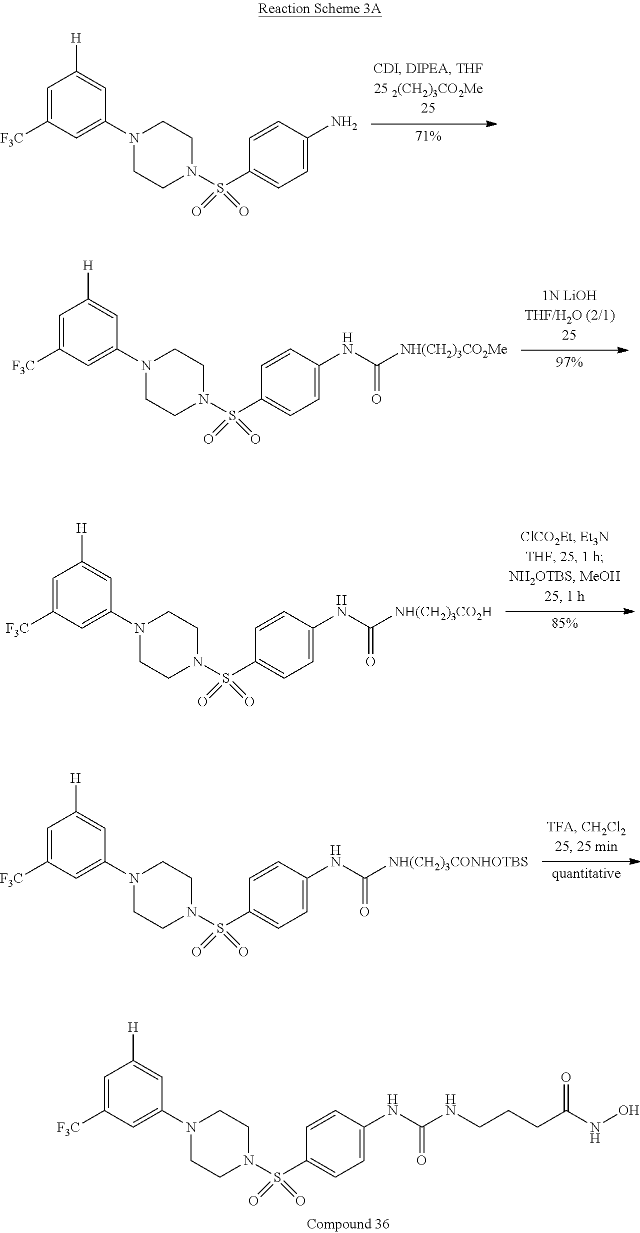

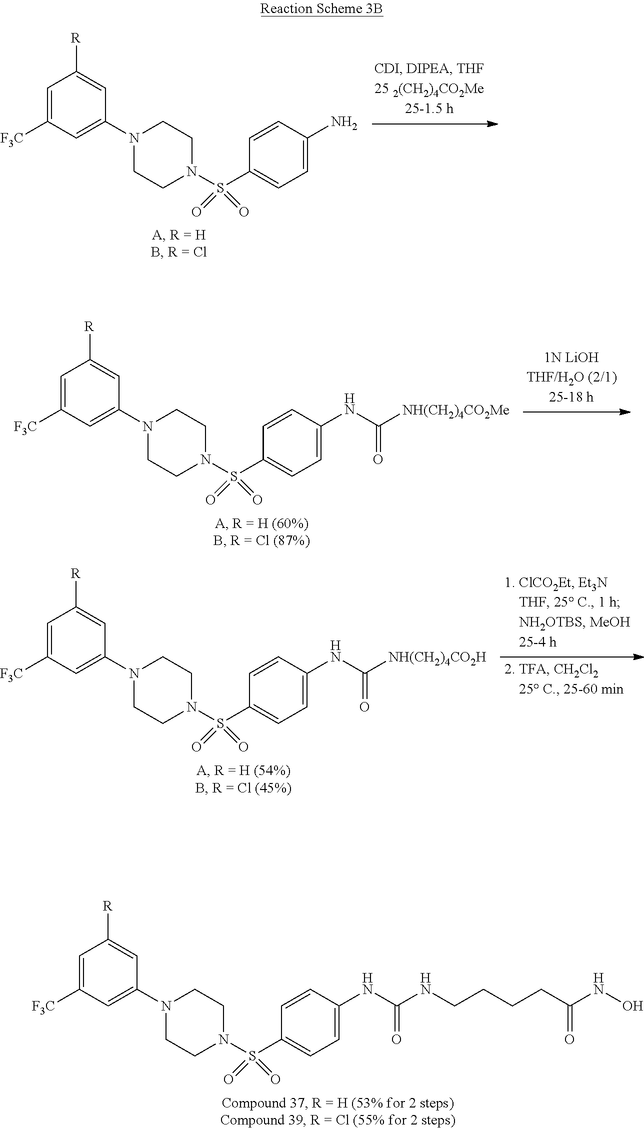

### Example 9: Biochemical Characterization of the LpxH Targeting Analog Compounds

The analog compounds synthesized above were biochemically characterized for K. pneumoniae LpxH inhibition at 0.1 μM using the nonradioactive, colorimetric malachite green assay. The specific activity of K. pneumoniae LpxH in the presence of the LpxH targeting analog compounds are shown in Table 1, below.

TABLE 1

| | 0.1 μM compound | | |
|---|---|---|---|
| | Activity | Per- | Per- |
| | (μmol/min/ | centage | centage |
| Structure | mg)^{a} | Activity | Inhibition |
| DMSO | 298.7 ± 9.0 | 100 ± 3 | 0 |
| 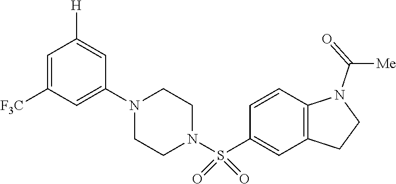 [SMILES: CC(=O)N1CCc2cc(S(=O)(=O)N3CCN(c4cccc(C(F)(F)F)c4)CC3)ccc21] | | 78^{b} | 22 |
| 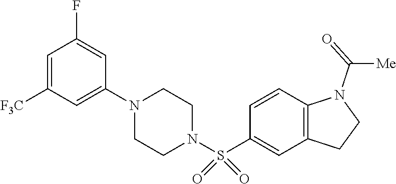 [SMILES: CC(=O)N1CCc2cc(S(=O)(=O)N3CCN(c4cc(F)cc(C(F)(F)F)c4)CC3)ccc21] | | 52^{b} | 48 |
| 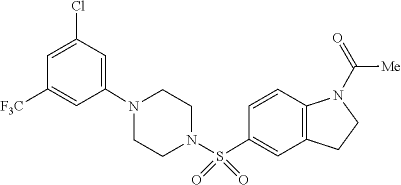 [SMILES: CC(=O)N1CCc2cc(S(=O)(=O)N3CCN(c4cc(Cl)cc(C(F)(F)F)c4)CC3)ccc21] | | 21^{b} | 79 |
| 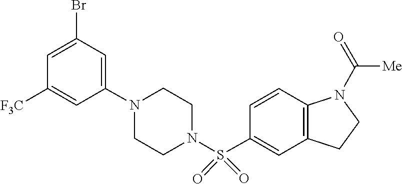 [SMILES: CC(=O)N1CCc2cc(S(=O)(=O)N3CCN(c4cc(Br)cc(C(F)(F)F)c4)CC3)ccc21] | 128.3 ± 12 | 43 ± 4 | 57 |
| 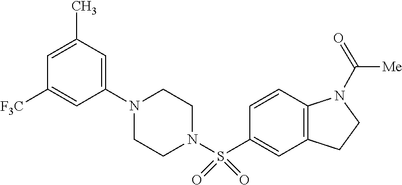 [SMILES: CC(=O)N1CCc2cc(S(=O)(=O)N3CCN(c4cc(C)cc(C(F)(F)F)c4)CC3)ccc21] | 148.8 ± 9.3 | 50 ± 3 | 50 |
| 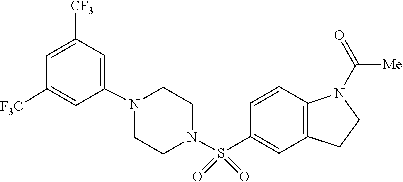 [SMILES: CC(=O)N1CCc2cc(S(=O)(=O)N3CCN(c4cc(C(F)(F)F)cc(C(F)(F)F)c4)CC3)ccc21] | 252.1 ± 38.9 | 84 ± 13 | 16 |
| 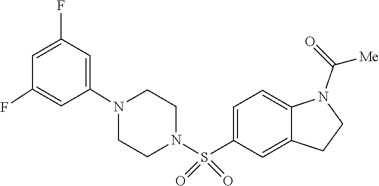 [SMILES: CC(=O)N1CCc2cc(S(=O)(=O)N3CCN(c4cc(F)cc(F)c4)CC3)ccc21] | 182.4 ± 16.7 | 61 ± 6 | 39 |
| 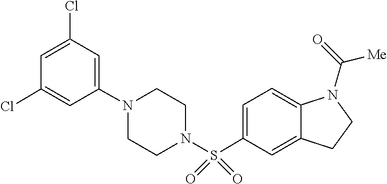 [SMILES: CC(=O)N1CCc2cc(S(=O)(=O)N3CCN(c4cc(Cl)cc(Cl)c4)CC3)ccc21] | 121.7 ± 25.1 | 41 ± 8 | 59 |
| 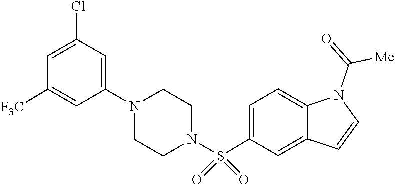 [SMILES: CC(=O)n1ccc2cc(S(=O)(=O)N3CCN(c4cc(Cl)cc(C(F)(F)F)c4)CC3)ccc21] | 156.1 ± 15.2 | 52 ± 5 | 48 |
| 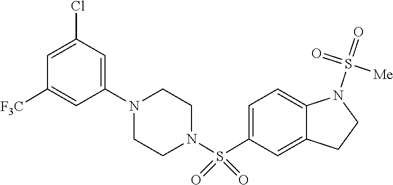 [SMILES: CS(=O)(=O)N1CCc2cc(S(=O)(=O)N3CCN(c4cc(Cl)cc(C(F)(F)F)c4)CC3)ccc21] | 262.7 ± 34.7 | 88 ± 12 | 12 |
| 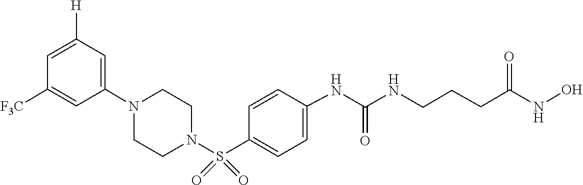 [SMILES: O=C(CCCNC(=O)Nc1ccc(S(=O)(=O)N2CCN(c3cccc(C(F)(F)F)c3)CC2)cc1)NO] | 253.3 ± 20.0 | 85 ± 7 | 15 |
| 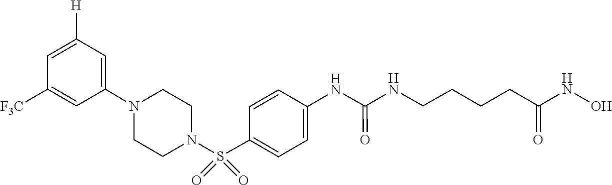 [SMILES: O=C(CCCCNC(=O)Nc1ccc(S(=O)(=O)N2CCN(c3cccc(C(F)(F)F)c3)CC2)cc1)NO] | 210.0 ± 26.3 | 70 ± 9 | 30 |
| 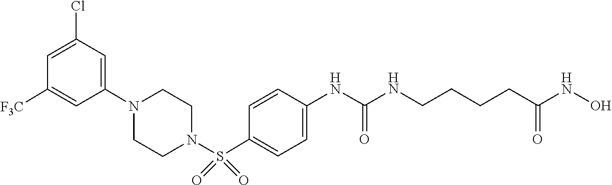 [SMILES: O=C(CCCCNC(=O)Nc1ccc(S(=O)(=O)N2CCN(c3cc(Cl)cc(C(F)(F)F)c3)CC2)cc1)NO] | 107.6 ± 15.6 | 36 ± 5 | 64 |

Among the phenyl group analogs of Compound 31 with a second m-substituent of the m-trifluoromethyl substituted phenyl ring (e.g., m-hydrogen substituted analog Compound 1, m-fluoro substituted analog Compound 26, m-methyl substituted analog 1-(5-((4-(3-methyl-5-(trifluoromethyl)phenyl)piperazin-1-yl)sulfonyl)indolin-1-yl)ethan-1-one, and m-chloro substituted analog Compound 31), there is a general trend of increasing potency following the increase in the volume of substituents (H<F<CH₃<Cl), except for m-bromo substituted analog -(5-((4-(3-Bromo-5-(trifluoromethyl)phenyl)piperazin-1-yl)sulfonyl)indolin-1-yl)ethan-1-one and m-trifluoromethyl substituted analog Compound 41.

A similar trend was also observed when both m-positions are substituted with fluoro (1-(5-((4-(3,5-Difluorophenyl)piperazin-1-yl)sulfonyl)indolin-1-yl)ethan-1-one), chloro (Compound 42), and trifluoromethyl (1-(5-((4-(3,5-Bis(trifluoromethyl)phenyl)piperazin-1-yl)sulfonyl)indolin-1-yl)ethan-1-on) groups, with the dichloro substituted analog Compound 42 displaying better activity (59% inhibition) than the difluoro substituted analog 1-(5-((4-(3,5-Difluorophenyl)piperazin-1-yl)sulfonyl)indolin-1-yl)ethan-1-one (39% inhibition) and the difluoromethyl substituted analog Compound 41 (16% inhibition).

Replacement of the indoline of Compound 31 with indole (Compound 40) slightly decreased the potency. When the N-acetyl group of the indoline ring of Compound 31 was replaced with a methanesulfonyl group (5-((4-(3-Chloro-5-(trifluoromethyl)phenyl)piperazin-1-yl)sulfonyl)-1-(methylsulfonyl)indoline), the activity dropped to 12% inhibition, indicating that the N-acetyl group of Compound 31 is critical to the LpxH inhibition.

Three compounds-namely, N-Hydroxy-4-(3-(4-((4-(3-(trifluoromethyl)phenyl)piperazin-1-yl)sulfonyl)phenyl)ureido)butanamide (Compound 36), N-Hydroxy-5-(3-(4-((4-(3-(trifluoromethyl)phenyl)piperazin-1-yl)sulfonyl)phenyl)ureido)pentanamide (Compound 37), N-Hydroxy-5-(3-(4-((4-(3-chloro-5-(trifluoromethyl)phenyl)piperazin-1-yl)sulfonyl)phenyl)ureido)-pentanamide (Compound 39)—were synthesized containing different lengths of acyl chains with a terminal hydroxamate group designed to chelate the di-manganese cluster in the active site. The shorter N-acyl chain analog (Compound 36) showed slightly lower activity than Compound 1, whereas extending the acyl chain of Compound 36 by one methylene group (Compound 37) improved the potency over Compound 1.

Combining the long acyl chain with the phenyl ring doubly substituted with trifluoromethyl and chloro groups yielded the most active compound of Table 1. Despite the excellent in vitro activity of Compound 39, its long acyl chain with many rotatable bonds may have negatively impacted the antibiotic activity of such compound, yielding a MIC of 32 μg/mL against K. pneumoniae. The MIC value Compound 39 was still an improvement over Compound 1 (MIC>64 μg/mL), but is significantly worse than Compound 31 (1.6 μg/mL). Such a result was attributed to the poor membrane permeability of Compound 39 due to the highly flexible and hydrophobic nature of the extended acyl chain of Compound 39.

### Example 10: Structure of K. pneumoniae LpxH in Complex with N-Hydroxy-5-(3-(4-((4-(3-chloro-5-(trifluoromethyl)phenyl)piperazin-1-yl)sulfonyl)phenyl)ureido)-pentanamide (Compound 39)

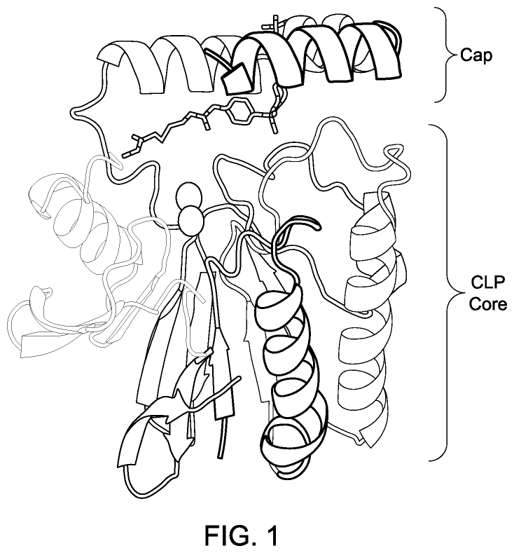

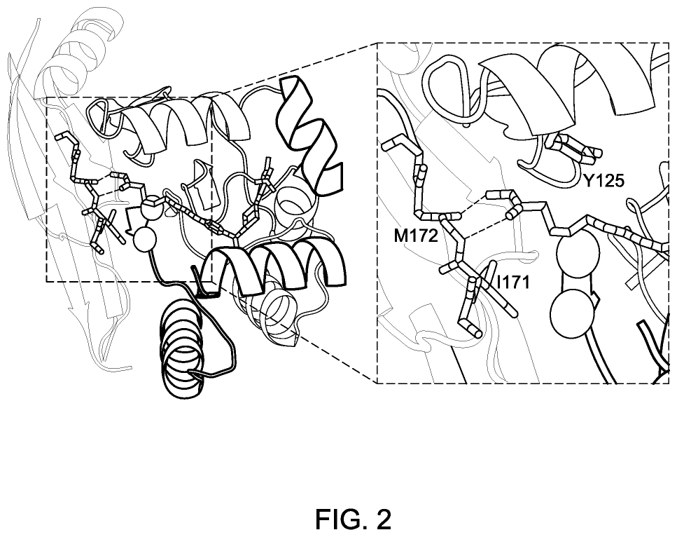

The co-crystal structure of the K. pneumoniae LpxH/N-Hydroxy-5-(3-(4-((4-(3-chloro-5-(trifluoromethyl)phenyl)piperazin-1-yl)sulfonyl)phenyl)ureido)-pentanamide (Compound 39) complex was determined at 1.85 Å to assess the interaction of such compound with K. pneumoniae Lpx. FIGS. 1 and 2 are images of the KpLpxH and Compound 39 complex, with LpxH is shown in the cartoon model and the catalytic di-manganese cluster shown in the sphere model. The hydrogen bonds between the hydroxamate group of Compound 39 and the backbone of M172 of LpxH are indicated by dashed lines. The results of the Data collection and statistics of the K. pneumoniae LpxH/Compound 39 Complex are provided in Table 2, below.

TABLE 2

| | K. pneumoniae LpxH/27b | |
|---|---|---|
| | Complex | |
| PDB | 6WII | |
| Data collection | | |
| Wavelength (Å) | 0.9791 | |
| Space group | P 32 2 1 | |
| Cell dimensions | | |
| a, b, c (Å) | 105.71, 105.71, 53.65 | |
| α, β, γ (°) | 90, 90, 120 | |
| Resolution (Å) | 46.29-1.85 | |
| | (1.90-1.85) | |
| Rₘₑₐₛ | 0.092 | (0.863) |
| Mean I/σI | 16.88 | (3.75) |
| Completeness (%) | 99.9 | (100.0) |
| Redundancy | 10.97 | (11.42) |
| Total reflections | 326145 | (24632) |
| Unique reflections | 29725 | (2156) |

| | |
|---|---|
| Refinement | |
| R_{work}/R_{free} | 0.1603/0.1881 |
| No. atoms | 2171 |
| Protein | 1953 |
| Ligand/ion | 64 |
| Water | 154 |
| Average B-factors | 33.60 |
| Protein | 32.24 |
| Ligand/ion | 47.47 |
| Water | 44.99 |
| R.m.s. deviations | |
| Bond lengths (Å) | 0.007 |
| Bond angles (°) | 0.87 |
| Ramachandran | |
| a. Favored (%) | 97.86 |
| b. Allowed (%) | 2.14 |
| c. Outliers (%) | 0 |

It was determined that Compound 39 occupies the hydrophobic substrate-binding chamber between the calcineurin-like phosphatase (CLP) domain and the insertion cap domain (see FIG. 1). However, its N-acyl chain snakes into the active site as designed: the acyl chain picks up additional hydrophobic interactions with Y125 of the insertion cap and I171 on the loop connecting the cap back to the CLP core domain (see FIG. 2).

Most unexpectedly, the hydroxamate group did not chelate the di-manganese cluster as we designed; instead, its carbonyl group and N-hydroxyl group form two hydrogen bonds with the backbone amide and carbonyl group of M172 of the same loop as I171 (see FIG. 2). Although these hydroxamate-containing acyl chains did not reach the metal cluster as one of ordinary skill would predict, they demonstrate the feasibility to expand molecular interactions into the active site of LpxH.

Overall, it was surprisingly discovered that there was a correlation for certain LpxH targeting analog compounds between the compound activity and the volume of the functional groups at the meta-position of the trifluoromethyl substituted distal phenyl ring, with the compound potency increasing from H, F, CH₃ and achieving maximal activity at Cl substitution. A similar trend was observed with the symmetrical substitutions at the meta-positions of the distal phenyl ring of certain LpxH targeting analog compounds. In particular, Compound 39 occupies a previously untapped pocket near the di-manganese cluster in the LpxH active site. Despite having a proximal phenyl group instead of an indoline group, the acyl chain extension of Compound 39 into the active site unexpectedly restores much of the in vitro activity to almost equal to that of Compound 31 with an indoline group.

### Example 11: Cloning and Purification of E. coli LpxH

The Escherichia coli LpxH gene was cloned into a modified pET30 vector (EMD Millipore) containing an N-terminal His₁₀-SUMO-fusion protein. The sequence verified plasmid was used to transform BL21(DE3)STAR competent E. coli cells (ThermoFisher). Cells were grown in the Luria Broth media at 37° C. until OD₆₀₀ reached 0.5, induced with 1 mM isopropyl-β-D-thiogalactopyranoside (IPTG) for 3 hours, and then harvested by centrifugation.

All of the purification procedures were carried out at 4° C. Cells from 8 L of induced culture were resuspended and lysed in 120 mL of the lysis buffer containing 20 mM HEPES (pH 8.0) and 200 mM NaCl using French Press. Cell debris were removed by centrifugation at 10,000×g for 40 minutes. To the supernatant, n-dodecyl-β-D-maltopyranoside (DDM) was added to reach a final concentration of 1.5% (w/v; 29 mM). After 2 hours of incubation, membranes were removed by centrifugation at 100,000×g for 1 hour. Supernatant from the centrifugation was diluted to a final volume of 240 mL with the lysis buffer and added to a column containing 20 mL of HisPur™ Ni-NTA resin (ThermoFisher) pre-equilibrated with 100 mL of the purification buffer containing 20 mM HEPES (pH 8.0), 200 mM NaCl, and 0.0174% (w/v; 0.34 mM) DDM. The column was washed with 250 mL of the purification buffer containing 50 mM imidazole, and the His₁₀-SUMO-LpxH was eluted with 150 mL of the purification buffer containing 300 mM imidazole. The eluted protein sample was concentrated and further purified with size-exclusion chromatography (Superdex 200; GE Healthcare Life Sciences) in the purification buffer.

### Example 12: ³²P-Autoradiographic TLC Assay for AaLpxE

The ³²P-radiolabeled and unlabeled lipid X and the wild-type and H149Q AaLpxE were prepared according to methods known by one of ordinary skill and the teachings disclosed herein. To investigate the AaLpxE activity toward lipid X as the substrate, a 10 μL reaction mixture containing 50 mM Tris-HCl (pH 7.5), 0.05% Triton X-100, 100 μM lipid X and 500 cpm/μL ³²P-lipid X was pre-incubated at 30° C., and the reaction was initiated by addition of 1 μg/mL purified AaLpxE. The reactions were quenched by spotting 2 μL reaction mixture on the TLC plate at the specified time points. The plate was dried and developed in a solvent system consisting of chloroform, methanol, acetic acid, and water (25:15:4:4, v/v), followed by analysis using the Typhoon FLA 7000 PhosphorImager scanner equipped with ImageQuant software (GE Healthcare).

To examine the compatibility of the malachite green assay kit (SIGMA ALDRICH catalog number MAK307) with our assay condition, the phosphate standard provided with the kit was diluted into our assay reaction buffer containing 20 mM Tris-HCl pH 8.0, 0.5 mg/mL BSA, 0.02% Triton X-100, and 1 mM MnCl₂. The linear colorimetric response to a range of phosphate concentrations up to 200 μM was confirmed.

The autoradiographic assay protocol for LpxH were adapted based on the teachings herein and knowledge of one of ordinary skill, including the modification of using the current assay buffer conditions as described above and the reaction incubation temperature at 37° C. instead of 30° C. Further discussion regarding LpxH activity assays is discussed in Cho, J., Lee, M., Cochrane, C. S., Webster, C. G., Fenton, B. A., Zhao, J., Hong, J., Zhou, P. (2020) Structural basis of the UDP-diacylglucosamine pyrophosphohydrolase LpxH inhibition by sulfonyl piperazine antibiotics. Proc. Natl. Acad. Sci. U.S.A. 117 (8) 4109-4116, https://doi.org/10.1073/pnas.1912876117.

A typical assay for LpxH using the coupled malachite green assay protocol contained the assay reaction buffer (20 mM Tris-HCl pH 8.0, 0.5 mg/mL BSA, 0.02% Triton X-100, and 1 mM MnCl₂) with 100 μM UDP-DAGn and 5% DMSO or inhibitors. The reaction mixtures were pre-incubated at 37° C. for 10 minutes before LpxH was added with a 5-fold dilution to start the reaction at 37° C. At the desired reaction time points, an aliquot of 20 μL reaction was removed and added to a well in 96-well half-area plate containing 5 mM EDTA (final concentration) to quench the LpxH reaction. Purified AaLpxE was then added to a final concentration of 5 μg/mL. The plate was incubated at 37° C. for 30 minutes followed by addition of formic acid to a final concentration of 3.75 M to quench the reaction. The malachite green reagent was added with a 5-fold dilution and the absorbance at 620 nm was measured after 30-minute incubation at room temperature. The amount of free inorganic phosphate from hydrolyzed lipid X was determined from the phosphate standard curve and used to calculate the specific activity of the SUMO-LpxH fusion protein.

For determination of IC₅₀, up to 80 μM of Compound 1 compound was added to the assay reaction containing 5% DMSO. Our preliminary analysis showed that despite the strong inhibition of LpxH activity by Compound 1 at 1 μM, there existed significant levels of enzymatic activity at elevated compound concentrations beyond 10 μM. Therefore, the protocol for IC₅₀ determination was adjusted to include 10% DMSO instead of 5% DMSO for the dose response analysis of Compound 1 to mitigate the concern of limited compound solubility. The increase of DMSO concentration had minimal impact on the specific activity of LpxH. The IC₅₀ value was extracted from fitting of the dose response curve of vᵢ/v₀=1/(1+[I]/IC₅₀).

### Example 13: Pharmacophore Modeling

The pharmacophore model of LpxH targeting compounds was generated based on total number of 22 analogs that were tested for their inhibition effects against LpxH (see Table 3, below). The molecular structures were sketched and built with Maestro. 11.2 (Schrödinger, NY). The pharmacophore model was generated with the “Develop Pharmacophore Model” module of Phase. Low energy conformations of LpxH inhibitors were generated by LigPrep of Schrodinger. The pharmacophore model was developed with the most active training set compounds, which are defined as “active ligands” for pharmacophore generation. Features of hydrogen bond acceptor and donor, hydrophobic, negative, positive, and aromatic rings were located in the pharmacophore model. Pharmacophores with tree features that match to all active ligands were generated by using a tree-based partitioning technique (specifically, Phase, version 11.2; Schrödinger) with maximum tree depth of five. The generated pharmacophore hypotheses were scored with default parameters. The specific activity of E. coli LpxH in the presence of certain LpxH targeting analog compounds.

TABLE 3

| | | 10 μM compound | | 1 μM compound | |
|---|---|---|---|---|---|
| Com- | | Activity | Per- | Activity | Per- |
| pound | | (μmol/ | centage | (μmol/ | centage |
| Nos. | Structure | min/mg)^{a} | Activity | min/mg) | Activity |
| DMSO | | 96.0 ± 4.7 | N/A | 96.0 ± 4.7 | N/A |
| 1 | 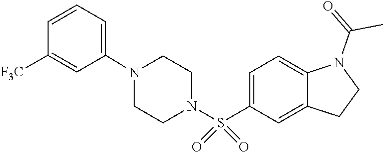 [SMILES: CC(=O)N1CCc2cc(S(=O)(=O)N3CCN(c4cccc(C(F)(F)F)c4)CC3)ccc21] | 5.7 ± 2.5 | 6 ± 3 | 16.0 ± 0.7 | 17 ± 1 |
| 4 | 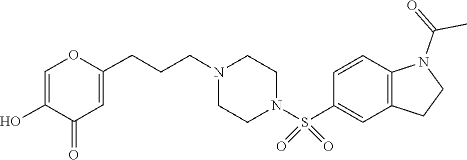 [SMILES: CC(=O)N1CCc2cc(S(=O)(=O)N3CCN(CCCc4cc(=O)c(O)co4)CC3)ccc21] | 109.5 ± 5.0 | 114 ± 8 | ND | ND |
| 5 | 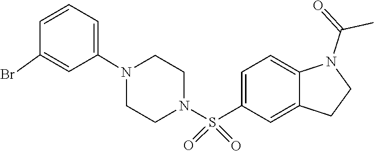 [SMILES: CC(=O)N1CCc2cc(S(=O)(=O)N3CCN(c4cccc(Br)c4)CC3)ccc21] | 22.6 ± 5.8 | 24 ± 6 | 25.3 ± 8.0 | 26 ± 8 |
| 6 | 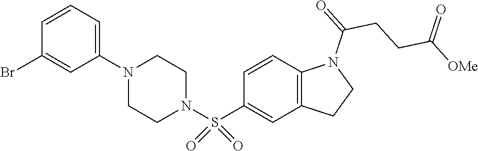 [SMILES: COC(=O)CCC(=O)N1CCc2cc(S(=O)(=O)N3CCN(c4cccc(Br)c4)CC3)ccc21] | 40.0 ± 7.9 | 42 ± 8 | 50.6 ± 10.5 | 53 ± 11 |
| 8 | 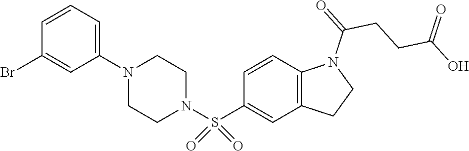 [SMILES: O=C(O)CCC(=O)N1CCc2cc(S(=O)(=O)N3CCN(c4cccc(Br)c4)CC3)ccc21] | 23.5 ± 7.0 | 24 ± 7 | 50.0 ± 10.7 | 52 ± 11 |
| 9 | 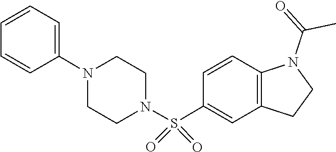 [SMILES: CC(=O)N1CCc2cc(S(=O)(=O)N3CCN(c4ccccc4)CC3)ccc21] | 31.1 ± 8.2 | 32 ± 9 | 105.7 ± 9.1 | 110 ± 11 |
| 9 | 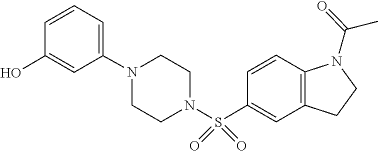 [SMILES: CC(=O)N1CCc2cc(S(=O)(=O)N3CCN(c4cccc(O)c4)CC3)ccc21] | 87.1 ± 14.1 | 91 ± 15 | ND | ND |
| 10 | 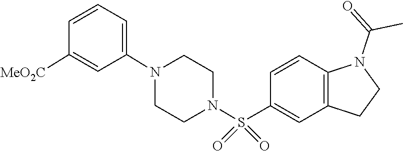 [SMILES: COC(=O)c1cccc(N2CCN(S(=O)(=O)c3ccc4c(c3)CCN4C(C)=O)CC2)c1] | 34.3 ± 6.7 | 36 ± 7 | 98.5 ± 6.3 | 103 ± 8 |
| 11 | 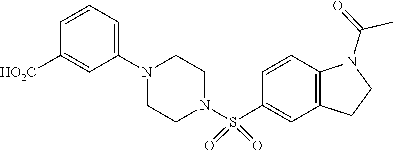 [SMILES: CC(=O)N1CCc2cc(S(=O)(=O)N3CCN(c4cccc(C(=O)O)c4)CC3)ccc21] | 125.5 ± 6.7 | 131 ± 9 | ND | ND |
| 13 | 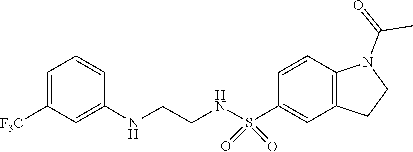 [SMILES: CC(=O)N1CCc2cc(S(=O)(=O)NCCNc3cccc(C(F)(F)F)c3)ccc21] | 142.6 ± 13.8 | 149 ± 16 | ND | ND |
| 14 | 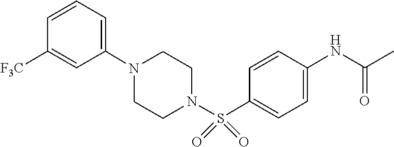 [SMILES: CC(=O)Nc1ccc(S(=O)(=O)N2CCN(c3cccc(C(F)(F)F)c3)CC2)cc1] | 28.6 ± 10.8 | 30 ± 11 | 67.3 ± 7.1 | 70 ± 8 |
| 15 | 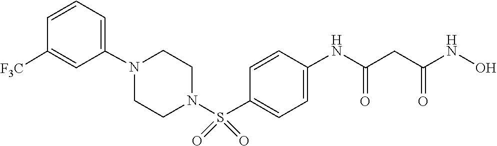 [SMILES: O=C(CC(=O)Nc1ccc(S(=O)(=O)N2CCN(c3cccc(C(F)(F)F)c3)CC2)cc1)NO] | 52.8 ± 25.5 | 55 ± 27 | ND | ND |
| 18 | 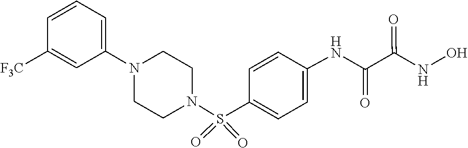 [SMILES: O=C(NO)C(=O)Nc1ccc(S(=O)(=O)N2CCN(c3cccc(C(F)(F)F)c3)CC2)cc1] | 38.9 ± 8.8 | 41 ± 9 | 116.6 ± 1.4 | 121 ± 6 |
| 17 | 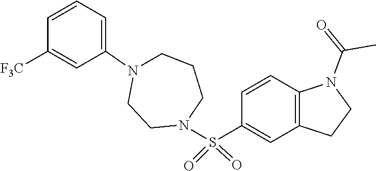 [SMILES: CC(=O)N1CCc2cc(S(=O)(=O)N3CCCN(c4cccc(C(F)(F)F)c4)CC3)ccc21] | 89.9 ± 19.7 | 94 ± 21 | ND | ND |
| 18 | 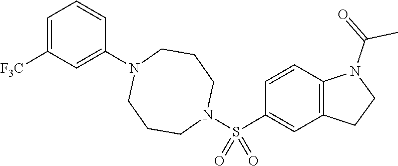 [SMILES: CC(=O)N1CCc2cc(S(=O)(=O)N3CCCN(c4cccc(C(F)(F)F)c4)CCC3)ccc21] | 143.3 ± 12.3 | 149 ± 15 | ND | ND |
| 19 | 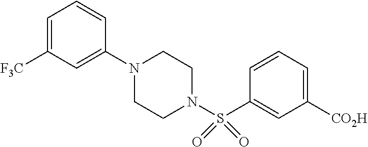 [SMILES: O=C(O)c1cccc(S(=O)(=O)N2CCN(c3cccc(C(F)(F)F)c3)CC2)c1] | 93.9 ± 5.3 | 98 ± 7 | ND | ND |
| 20 | 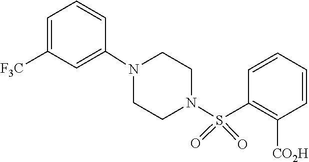 [SMILES: O=C(O)c1ccccc1S(=O)(=O)N1CCN(c2cccc(C(F)(F)F)c2)CC1] | 103.9 ± 11.7 | 108 ± 13 | ND | ND |
| 43 | 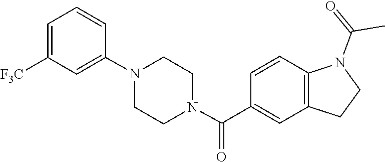 [SMILES: CC(=O)N1CCc2cc(C(=O)N3CCN(c4cccc(C(F)(F)F)c4)CC3)ccc21] | 98.0 ± 10.9 | 102 ± 12 | ND | ND |
| 21 | 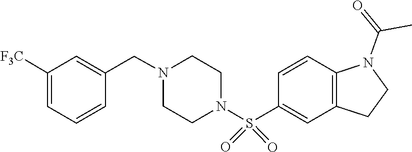 [SMILES: CC(=O)N1CCc2cc(S(=O)(=O)N3CCN(Cc4cccc(C(F)(F)F)c4)CC3)ccc21] | 83.3 ± 2.5 | 87 ± 5 | ND | ND |
| 22 | 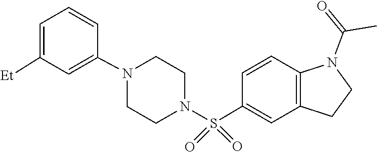 [SMILES: CCc1cccc(N2CCN(S(=O)(=O)c3ccc4c(c3)CCN4C(C)=O)CC2)c1] | 20.5 ± 1.6 | 21 ± 2 | 64.9 ± 7.8 | 68 ± 9 |
| 23 | 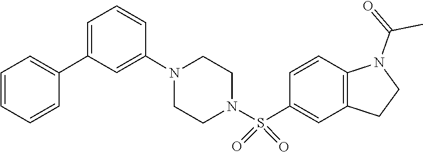 [SMILES: CC(=O)N1CCc2cc(S(=O)(=O)N3CCN(c4cccc(-c5ccccc5)c4)CC3)ccc21] | 23.0 ± 2.9 | 24 ± 3 | 42.9 ± 9.8 | 45 ± 10 |
| 24 | 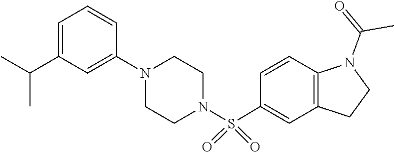 [SMILES: CC(=O)N1CCc2cc(S(=O)(=O)N3CCN(c4cccc(C(C)C)c4)CC3)ccc21] | 28.2 ± 2.5 | 29 ± 3 | 66.8 ± 2.1 | 70 ± 4 |

### Example 13: Synthesis of LpxH Inhibitors Compound 1 Analogs Toward E. coli LpxH

General chemistry procedures. All reactions were conducted in oven-dried glassware under nitrogen. Unless otherwise stated all reagents were purchased from Sigma-Aldrich, Acros, or Fisher and were used without further purification. All solvents were ACS grade or better and used without further purification except tetrahydrofuran (THF) which was freshly distilled from sodium/benzophenone each time before use. Analytical thin layer chromatography (TLC) was performed with glass backed silica gel (60 Å) plates with fluorescent indication (Whatman). Visualization was accomplished by UV irradiation at 254 nm and/or by staining with p-anisaldehyde solution. Flash column chromatography was performed by using silica gel (particle size 230-400 mesh, 60 Å). All ¹H NMR spectra were recorded with a Varian 400 (400 MHz) spectrometer in CDCl₃, (CD₃)₂CO, and CD₃CN. All NMR δ values are given in ppm, and all J values are in Hz. Electrospray ionization (ESI) mass spectrometry (MS) were recorded with an Agilent 1100 series (LC/MSD trap) spectrometer and were performed to obtain the molecular masses of the compounds.

### Example 13A

1-(5-((4-(3-Bromophenyl)piperazin-1-yl)sulfonyl)indolin-1-yl)ethan-1-one (Compound 5), the structure of which is shown below, was synthesized according to the following procedure.

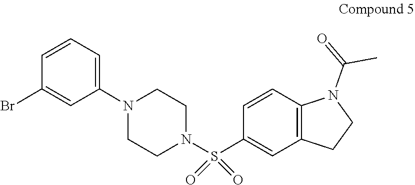 [SMILES: CC(=O)N1CCc2cc(S(=O)(=O)N3CCN(c4cccc(Br)c4)CC3)ccc21]

To a solution of 1-(3-bromophenyl)piperazine (26 mg, 0.1 mmol) in 1,4-dioxane (1 mL, 0.1 M) Et₃N (0.02 mL, 0.12 mmol) was added. The resulting reaction mixture was heated until the temperature reached 60° C. 1-Acetyl-5-indolinesulfonyl chloride (26 mg, 0.1 mmol) in 1,4-dioxane (0.5 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄, and concentrated in vacuo. Recrystallization from hot 2-propanol resulted in Compound 5 (34 mg, 74%): ¹H NMR (400 MHz, CDCl₃) δ 8.33 (d, J=8.4 Hz, 1H), 7.62 (dd, J=8.4, 2.0 Hz, 1H), 7.56 (d, J=2.0 Hz, 1H), 7.09 (t, J=8.4 Hz, 1H), 6.98 (m, 2H), 6.77 (m, 1H), 4.15 (t, J=8.4 Hz, 2H), 3.29-3.23 (m, 6H), 3.13 (m, 4H), 2.26 (s, 3H); HRMS (ESI) m/z 464.0630 [(M+H)⁺, C₂₀H₂₂BrN₃O₃S requires 464.0638].

### Example 13B

1-(5-((4-Phenylpiperazin-1-yl)sulfonyl)indolin-1-yl)ethan-1-one (Compound 8), the structure of which is shown below, was synthesized according to the following procedure.

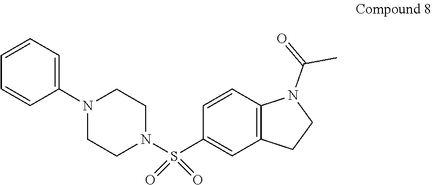 [SMILES: CC(=O)N1CCc2cc(S(=O)(=O)N3CCN(c4ccccc4)CC3)ccc21]

To a solution of (32 mg, 0.2 mmol) in 1,4-dioxane (1 mL, 0.2 M) Et₃N (0.033 mL, 0.24 mmol) was added and the reaction mixture was heated until the temperature reached 60° C. 1-Acetyl-5-indolinesulfonyl chloride (52 mg, 0.2 mmol) in 1,4-dioxane (0.5 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄, and concentrated in vacuo. Recrystallization from hot 2-propanol resulted in Compound 8 (35 mg, 45%): ¹H NMR (400 MHz, CDCl₃) δ 8.34 (d, J=8.4 Hz, 1H), 7.63 (dd, J=8.4, 2.0 Hz, 1H), 7.57 (s, 1H), 7.25 (m, 2H), 6.89 (m, 3H), 4.15 (t, J=8.4 Hz, 2H), 3.25 (m, 6H), 3.16 (m, 4H), 2.26 (s, 3H); HRMS (ESI) m/z 386.1535 [(M+H)⁺, C₂₀H₂₃N₃O₃S requires 386.1533].

### Example 13C

1-(5-((4-(3-Ethylphenyl)piperazin-1-yl)sulfonyl)indolin-1-yl)ethan-1-one (Compound 22), the structure of which is shown below, was synthesized according to the following procedure.

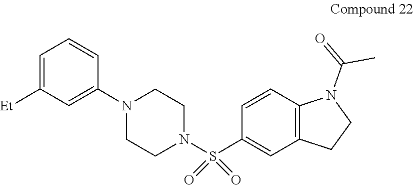 [SMILES: CCc1cccc(N2CCN(S(=O)(=O)c3ccc4c(c3)CCN4C(C)=O)CC2)c1]

To a solution of 1-(3-ethylphenyl)piperazine (13 mg, 0.068 mmol) in 1,4-dioxane (1 mL, 0.068 M), Et₃N (0.011 mL, 0.082 mmol) was added. The resulting reaction mixture was heated until the temperature reached 60° C. 1-Acetyl-5-indolinesulfonyl chloride (18 mg, 0.068 mmol) in 1,4-dioxane (0.5 mL) was added dropwise to the reaction mixture, which was stirred at 60° C. for 3 h, and then at 25° C. for 6 h. Water was added and the aqueous layer was extracted with EtOAc. The combined organic layers were dried over anhydrous Na₂SO₄, and concentrated in vacuo. The residue was purified by column chromatography (silica gel, CH₂Cl₂/MeOH, 40/1) to afford Compound 22 (15.7 mg, 54%): ¹H NMR (400 MHz, CDCl₃) δ 8.34 (d, J=8.8 Hz, 1H), 7.63 (d, J=8.8 Hz, 1H), 7.57 (s, 1H), 7.17 (t, J=7.2 Hz, 1H), 6.75 (d, J=7.2 Hz, 1H), 6.69 (m, 2H), 4.15 (t, J=8.8 Hz, 2H), 3.25 (m, 6H), 3.15 (m, 4H), 2.58 (q, J=7.6 Hz, 2H), 2.27 (s, 3H), 1.20 (t, J=7.6 Hz, 3H); HRMS (ESI) m/z 414.1846 [(M+H)⁺, C₂₂H₂₇N₃O₃S requires 414.1846].

### Example 13D

1-(5-((4-(3-Isopropylphenyl)piperazin-1-yl)sulfonyl)indolin-1-yl)ethan-1-one (Compound 24), the structure of which is shown below, was synthesized according to the following procedure.

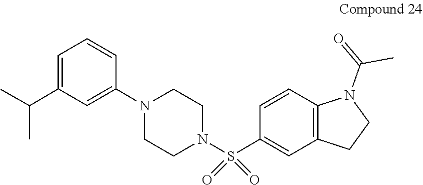 [SMILES: CC(=O)N1CCc2cc(S(=O)(=O)N3CCN(c4cccc(C(C)C)c4)CC3)ccc21]

Et₃N (0.014 mL, 0.1 mmol) was added to a solution of 1-(3-isopropylphenyl)piperazine (17 mg, 0.083 mmol) in 1,4-dioxane (1 mL, 0.083 M). The resulting reaction mixture was heated until the temperature reached 60° C. 1-Acetyl-5-indolinesulfonyl chloride (22 mg, 0.083 mmol) in 1,4-dioxane (0.5 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄, and concentrated in vacuo. The residue was purified by column chromatography (silica gel, CH₂Cl₂/MeOH, 40/1) to afford Compound 24 (16 mg, 46%): ¹H NMR (400 MHz, CDCl₃) δ 8.34 (d, J=8.4 Hz, 1H), 7.64 (d, J=8.4 Hz, 1H), 7.57 (s, 1H), 7.18 (t, J=7.6 Hz, 1H), 6.78 (d, J=7.6 Hz, 1H), 6.74 (s, 1H), 6.69 (d, J=8.0 Hz, 1H), 4.16 (t, J=8.4 Hz, 2H), 3.25 (m, 6H), 3.15 (m, 4H), 2.83 (m, 1H), 2.27 (s, 3H), 1.21 (d, J=7.2 Hz, 6H); HRMS (ESI) m/z 428.2003 [(M+H)⁺, C₂₃H₂₉N₃O₃S requires 428.2002].

1-(5-((4-([1,1′-Biphenyl]-3-yl)piperazin-1-yl)sulfonyl)indolin-1-yl)ethan-1-one (Compound 23), the structure of which is shown below, was synthesized according to the following procedure.

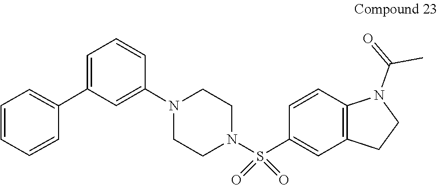 [SMILES: CC(=O)N1CCc2cc(S(=O)(=O)N3CCN(c4cccc(-c5ccccc5)c4)CC3)ccc21]

To a solution of 1-(3-phenylphenyl)piperazine (7 mg, 0.03 mmol) in 1,4-dioxane (0.5 mL, 0.06 M), Et₃N (0.005 mL, 0.036 mmol) was added. The resulting reaction mixture was heated until the temperature reached 60° C. 1-Acetyl-5-indolinesulfonyl chloride (8 mg, 0.03 mmol) in 1,4-dioxane (0.3 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄, and concentrated in vacuo. The residue was purified by column chromatography (silica gel, CH₂Cl₂/MeOH, 50/1) to afford Compound 23 (6 mg, 43%): ¹H NMR (400 MHz, CDCl₃) δ 8.35 (d, J=8.4 Hz, 1H), 7.65 (d, J=8.0 Hz, 1H), 7.58 (s, 1H), 7.53 (d, J=7.6 Hz, 2H), 7.42 (t, J=7.6 Hz, 2H), 7.33 (m, 2H), 7.11 (d, J=7.6 Hz, 1H), 7.06 (s, 1H), 6.86 (d, J=8.0 Hz, 1H), 4.16 (t, J=8.4 Hz, 2H), 3.30 (m, 6H), 3.17 (m, 4H), 2.27 (s, 3H); HRMS (ESI) m/z 462.1844 [(M+H)⁺, C₂₆H₂₇N₃O₃S requires 462.1846].

### Example 13E

1-(5-((4-(3-Hydroxyphenyl)piperazin-1-yl)sulfonyl)indolin-1-yl)ethan-1-one (Compound 9), the structure of which is shown below, was synthesized according to the following procedure.

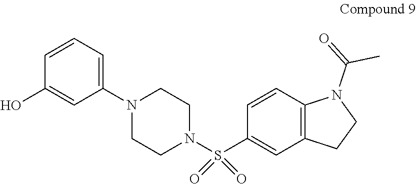 [SMILES: CC(=O)N1CCc2cc(S(=O)(=O)N3CCN(c4cccc(O)c4)CC3)ccc21]

To a solution of 1-(3-((tert-butyldimethylsilyl)oxy)phenyl)piperazine (33 mg, 0.11 mmol) in 1,4-dioxane (1 mL, 0.11 M) Et₃N (0.02 mL, 0.13 mmol) was added. The resulting reaction mixture was heated until the temperature reached 60° C. 1-Acetyl-5-indolinesulfonyl chloride (29 mg, 0.11 mmol) in 1,4-dioxane (0.5 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄, and concentrated in vacuo. The residue was purified by column chromatography (silica gel, hexanes/EtOAc, 1/1) to afford an a white intermediate powder (36 mg, 64%): ¹H NMR (400 MHz, CDCl₃) δ 8.32 (d, J=8.4 Hz, 1H), 7.61 (dd, J=8.4, 2.0 Hz, 1H), 7.55 (d, J=2.0 Hz, 1H), 7.10 (t, J=8.0 Hz, 1H), 6.46 (d, J=8.0 Hz, 1H), 6.35 (m, 2H), 4.14 (t, J=8.4 Hz, 2H), 3.26 (t, J=8.4 Hz, 2H), 3.20 (m, 4H), 3.13 (m, 4H), 2.26 (s, 3H), 0.95 (s, 9H), 0.16 (s, 6H). To a cooled (0° C.) solution of the intermediate (10 mg, 0.02 mmol) in THF (0.5 mL, 0.04 M) was added TBAF (1.0 M in THF, 0.022 mL, 0.022 mmol). The reaction mixture was stirred at 0° C. for 1 h. Water was added and extracted with EtOAc. The combined organic layer was washed with brine, dried over anhydrous Na₂SO₄, and concentrated in vacuo. The residue was purified by column chromatography (silica gel, CH₂Cl₂/MeOH, 10/1) to afford Compound 9 as a yellow oil (6 mg, 75%): ¹H NMR (400 MHz, CDCl₃) δ 8.34 (d, J=8.4 Hz, 1H), 7.63 (dd, J=8.4, 2.0 Hz, 1H), 7.56 (d, J=2.0 Hz, 1H), 7.11 (t, J=8.0 Hz, 1H), 6.48-6.41 (m, 3H), 4.16 (t, J=8.4 Hz, 2H), 3.30-3.19 (m, 10H), 2.67 (s, 3H); HRMS (ESI) m/z 402.1479 [(M+H)⁺, C₂₀H₂₃N₃O₄S requires 402.1482].

### Example 13F

3-(4-((1-Acetylindolin-5-yl)sulfonyl)piperazin-1-yl)benzoic acid (Compound 11), the structure of which is shown below, was synthesized according to the following procedure.

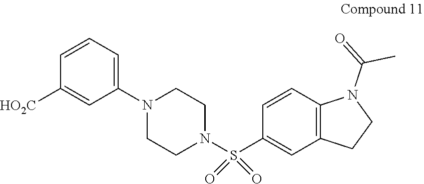 [SMILES: CC(=O)N1CCc2cc(S(=O)(=O)N3CCN(c4cccc(C(=O)O)c4)CC3)ccc21]

To a solution of 6b (34 mg, 0.15 mmol) in 1,4-dioxane (1.5 mL, 0.1 M) Et₃N (0.03 mL, 0.18 mmol) was added. The resulting reaction mixture was heated until the temperature reached 60° C. 1-Acetyl-5-indolinesulfonyl chloride (39 mg, 0.15 mmol) in 1,4-dioxane (0.5 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄, and concentrated in vacuo. The residue was purified by column chromatography (silica gel, CH₂Cl₂/MeOH, 20/1) to afford Compound 10 as a white powder (32 mg, 48%): ¹H NMR (400 MHz, CDCl₃) δ 8.33 (d, J=8.8 Hz, 1H), 7.62 (d, J=9.2 Hz, 1H), 7.56-7.52 (m, 3H), 7.30 (t, J=8.0 Hz, 1H), 7.03 (d, J=9.2 Hz, 1H), 4.15 (t, J=8.8 Hz, 2H), 3.88 (s, 3H), 3.30-3.25 (m, 6H), 3.15 (m, 4H), 2.26 (s, 3H); HRMS (ESI) m/z 444.1590 [(M+H)⁺, C₂₂H₂₅N₃O₅S requires 444.1588]; To a solution of Compound 10 (10 mg, 0.023 mmol) in THF (1 mL), MeOH (0.5 mL) and 1 N NaOH (0.23 mL, 0.23 mmol) were added. The reaction mixture was stirred at 25° C. for 15 h. 1 N HCl was added and the reaction mixture was extracted with EtOAc. The combined organic layers were washed with brine, dried over anhydrous Na₂SO₄, and concentrated in vacuo. The residue was purified by column chromatography (silica gel, CH₂Cl₂/MeOH, 10/1) to afford Compound 11 as a yellow oil (6 mg, 60%): ¹H NMR (400 MHz, (CD₃)₂CO) δ 8.30 (d, J=8.4 Hz, 1H), 7.62 (m, 1H), 7.56 (m, 1H), 7.50 (d, J=7.6 Hz, 1H), 7.33 (t, J=8.0 Hz, 1H), 7.20 (dd, J=8.4, 1.6 Hz, 1H), 4.26 (t, J=8.8 Hz, 2H), 3.34 (m, 4H), 3.13 (m, 4H), 2.22 (s, 3H); HRMS (ESI) m/z 430.1434 [(M+H)⁺, C₂₁H₂₃N₃O₅S requires 430.1431].

### Example 13G

N-(4-((4-(3-(Trifluoromethyl)phenyl)piperazin-1-yl)sulfonyl)phenyl)acetamide (Compound 14), the structure of which is shown below, was synthesized according to the following procedure.

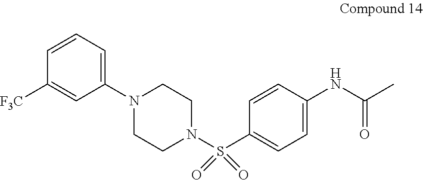 [SMILES: CC(=O)Nc1ccc(S(=O)(=O)N2CCN(c3cccc(C(F)(F)F)c3)CC2)cc1]

To a solution of 1-(3-(trifluoromethyl)phenyl)piperazine (500 mg, 2.17 mmol) in 1,4-dioxane (10 mL, 0.22 M), Et₃N (0.36 mL, 2.6 mmol) was added. The resulting reaction mixture was heated until the temperature reached 60° C. 4-Acetamidobenzenesulfonyl chloride (507 mg, 2.17 mmol) in 1,4-dioxane (2 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄, and concentrated in vacuo. Recrystallization from hot 2-propanol resulted in N-(4-((4-(3-(trifluoromethyl)phenyl)piperazin-1-yl)sulfonyl)phenyl)acetamide (668 mg, 72%): 1H NMR (400 MHz, CDCl₃) δ 7.76-7.70 (m, 4H), 7.39-7.34 (m, 2H), 7.15 (d, J=8.0 Hz, 1H), 7.09 (s, 1H), 7.06 (d, J=8.0 Hz, 1H), 3.31 (m, 4H), 3.20 (m, 4H), 2.23 (s, 3H); HRMS (ESI) m/z 428.1253 [(M+H)⁺, C₁₉H₂₀F₃N₃O₃S requires 428.1250].

### Example 13H

4-((4-(3-(Trifluoromethyl)phenyl)piperazin-1-yl)sulfonyl)aniline, the structure of which is shown below, was synthesized according to the following procedure.

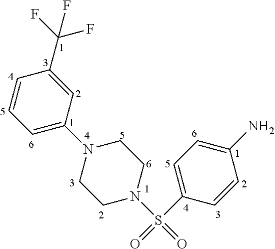 [SMILES: Nc1ccc(S(=O)(=O)N2CCN(c3cccc(C(F)(F)F)c3)CC2)cc1]

HCl (0.5 mL) was added to a solution of Compound 14 (200 mg, 0.47 mmol) in EtOH (1 mL). The resulting mixture was refluxed for 4 h, poured into ice-water, was made alkaline with 32% NH₄OH solution. The precipitate was filtered and recrystallization from EtOH resulted in the product 4-((4-(3-(trifluoromethyl)phenyl)piperazin-1-yl)sulfonyl)aniline as a white powder (120 mg, 66%): ¹H NMR (400 MHz, (CD₃)₂CO) δ 7.49 (d, J=8.4 Hz, 2H), 7.42 (t, J=8.4 Hz, 1H), 7.21 (s, 2H), 7.10 (d, J=8.0 Hz, 1H), 6.81 (d, J=8.4 Hz, 2H), 5.61 (bs, 2H), 3.35 (m, 4H), 3.07 (m, 4H); HRMS (ESI) m/z 386.1147 [(M+H)⁺, C₁₇H₁₈F₃N₃O₂S requires 386.1145]

### Example 13I

Ethyl-3-oxo-3-((4-((4-(3-(trifluoromethyl)phenyl)piperazin-1-yl)sulfonyl)phenyl)amino)propanoate, the structure of which is shown below, was synthesized according to the following procedure.

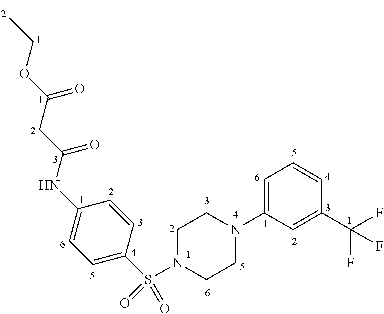 [SMILES: CCOC(=O)CC(=O)Nc1ccc(S(=O)(=O)N2CCN(c3cccc(C(F)(F)F)c3)CC2)cc1]

To a cooled (0° C.) solution of 4-((4-(3-(Trifluoromethyl)phenyl)piperazin-1-yl)sulfonyl)aniline (39 mg, 0.1 mmol), EDC (23 mg, 0.12 mmol), and HOBt (16 mg, 0.12 mmol) in CH₂Cl₂ (1 mL, 0.1 M), DIPEA (0.063 mL, 0.36 mmol) and mono-ethyl malonate (0.012 mL, 0.1 mmol) were added. The reaction mixture was stirred at 25° C. for 24 h and then water was added. The aqueous layer was extracted with CH₂Cl₂. The combined organic layers were subsequently washed with 1 N NaOH, 1 N HCl, and brine, dried over anhydrous Na₂SO₄, and concentrated in vacuo. The residue was purified by column chromatography (silica gel, hexanes/EtOAc, 2/1) to afford ethyl-3-oxo-3-((4-((4-(3-(trifluoromethyl)phenyl)piperazin-1-yl)sulfonyl)phenyl)amino)propanoate as a colorless oil (26 mg, 52%): ¹H NMR (400 MHz, CDCl₃) δ 9.70 (s, 1H), 7.79-7.74 (m, 4H), 7.34 (t, J=7.6 Hz, 1H), 7.11 (d, J=7.6 Hz, 1H), 7.05 (s, 1H), 7.00 (d, J=8.4 Hz, 1H), 4.28 (q, J=7.2 Hz, 2H), 3.50 (s, 2H), 3.28 (m, 4H), 3.18 (m, 4H), 1.33 (t, J=7.2 Hz, 3H); HRMS (ESI) m/z 500.1454 [(M+H)⁺, C₂₂H₂₄F₃N₃O₅S requires 500.1462].

### Example 13J

N¹-Hydroxy-N³-(4-((4-(3-(trifluoromethyl)phenyl)piperazin-1-yl)sulfonyl)phenyl)malonamide (Compound 15), the structure of which is shown below, was synthesized according to the following procedure.

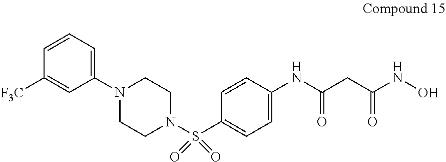 [SMILES: O=C(CC(=O)Nc1ccc(S(=O)(=O)N2CCN(c3cccc(C(F)(F)F)c3)CC2)cc1)NO]

To a solution of ethyl-3-oxo-3-((4-((4-(3-(Trifluoromethyl)phenyl)piperazin-1-yl)sulfonyl)phenyl)amino)propanoate (20 mg, 0.04 mmol) in MeOH (0.8 mL), a solution of LiOH·H₂O (8.4 mg, 0.2 mmol) in H₂O (0.2 mL) was added. The reaction mixture was stirred at 25° C. for 1 h and concentrated in vacuo. Water was added and the aqueous layer was washed with EtOAc, acidified with 1 N HCl. The aqueous layer was extracted with EtOAc and the combined organic layers were dried over anhydrous Na₂SO₄, and concentrated in vacuo. The residue was purified by column chromatography (silica gel, CH₂Cl₂/MeOH, 10/1) to afford the acid as a white powder (17.7 mg, 93%): ¹H NMR (400 MHz, CD₃OD) b 7.87-7.85 (m, 2H), 7.78-7.76 (m, 2H), 7.40-7.36 (m, 1H), 7.15 (m, 2H), 7.10-7.08 (m, 1H), 3.48 (s, 2H), 3.31 (m, 4H), 3.15 (m, 4H); HRMS (ESI) m/z 472.1151 [(M+H)⁺, C₂₀H₂₀F₃N₃O₅S requires 472.1149]. To a solution of the acid (16 mg, 0.034 mmol), DIPEA (0.012 mL, 0.068 mmol), and PyBOP (19 mg, 0.037 mmol) in DMF (1 mL) was added NH₂OTBS (5.4 mg, 0.037 mmol). The reaction mixture was stirred at 45° C. for 16 h and concentrated in vacuo. The residue was purified by column chromatography (silica gel, CH₂Cl₂/MeOH, 10/1) to afford Compound 15 as a white powder (6 mg, 35%): ¹H NMR (400 MHz, (CD₃)₂CO) δ 7.90 (d, J=8.8 Hz, 2H), 7.77 (d, J=8.8 Hz, 2H), 7.42 (m, 1H), 7.23-7.20 (m, 2H), 7.10 (d, J=7.6 Hz, 1H), 3.39 (m, 4H), 3.32 (s, 2H), 3.14 (m, 4H); HRMS (ESI) m/z 487.1251 [(M+H)⁺, C₂₀H₂₁F₃N₄O₅S requires 487.1258].

### Example 13K

2-Oxo-2-((4-((4-(3-(trifluoromethyl)phenyl)piperazin-1-yl)sulfonyl)phenyl)amino)acetic acid, the structure of which is shown below, was synthesized according to the following procedure.

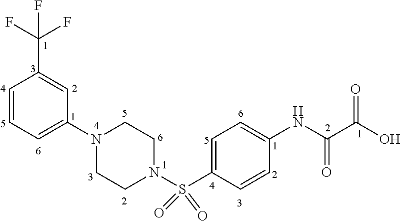 [SMILES: O=C(O)C(=O)Nc1ccc(S(=O)(=O)N2CCN(c3cccc(C(F)(F)F)c3)CC2)cc1]

Methyl chlorooxoacetate (0.02 mL, 0.18 mmol) was added dropwise to a cooled (0° C.) solution of 4-((4-(3-(Trifluoromethyl)phenyl)piperazin-1-yl)sulfonyl)aniline (56 mg, 0.15 mmol), Et₃N (0.025 mL, 0.18 mmol) in THF (1.5 mL, 0.1 M). The reaction mixture was stirred at 25° C. for 3 h. The reaction mixture was filtered to remove the ammonium salts, and the filtrate was washed with 2N HCl. The organic layer was dried over anhydrous Na₂SO₄ and concentrated in vacuo. The residue was purified by column chromatography (silica gel, hexanes/EtOAc, 2/1) to afford an intermediate as a white powder (60 mg, 85%): ¹H NMR (400 MHz, CDCl₃) δ 9.07 (s, 1H), 7.87-7.80 (m, 4H), 7.34 (t, J=8.0 Hz, 1H), 7.12 (d, J=7.6 Hz, 1H), 7.05 (s, 1H), 7.01 (d, J=8.4 Hz, 1H), 4.00 (s, 3H), 3.29 (m, 4H), 3.19 (m, 4H). To a solution of the intermediate (60 mg, 0.13 mmol) in MeOH (1.5 mL), a solution of LiOH·H₂O (27 mg, 0.65 mmol) in H₂O (0.5 mL) was added. The reaction mixture was stirred at 25° C. for 1 h and concentrated in vacuo. Water was added and the aqueous layer was washed with EtOAc and acidified with 1N HCl. The aqueous layer was extracted with EtOAc and the combined organic layers were dried over anhydrous Na₂SO₄ and concentrated in vacuo. The residue was purified by column chromatography (silica gel, CH₂Cl₂/MeOH, 10/1) to afford 2-oxo-2-((4-((4-(3-(trifluoromethyl)phenyl)piperazin-1-yl)sulfonyl)phenyl)amino)acetic acid as a white powder (53 mg, 90%): ¹H NMR (400 MHz, CDCl₃) δ 9.23 (s, 1H), 7.86 (m, 4H), 7.36 (t, J=8.0 Hz, 1H), 7.14 (d, J=8.0 Hz, 1H), 7.08 (s, 1H), 7.04 (d, J=8.4 Hz, 1H). 3.31 (m, 4H), 3.21 (m, 4H); HRMS (ESI) m/z 458.0989 [(M+H)⁺, C₁₉H₁₈F₃N₃O₅S requires 458.0992].

### Example 13L

N¹-Hydroxy-N²-(4-((4-(3-(trifluoromethyl)phenyl)piperazin-1-yl)sulfonyl)phenyl)oxalamide (Compound 16), the structure of which is shown below, was synthesized according to the following procedure.

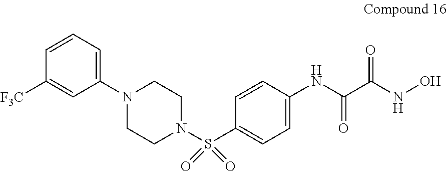 [SMILES: O=C(NO)C(=O)Nc1ccc(S(=O)(=O)N2CCN(c3cccc(C(F)(F)F)c3)CC2)cc1]

To a cooled (0° C.) solution of 2-oxo-2-((4-((4-(3-(trifluoromethyl)phenyl)piperazin-1-yl)sulfonyl)phenyl)amino)acetic acid (27 mg, 0.059 mmol), NMM (0.007 mL, 0.065 mmol) in THF (1 mL, 0.06 M), ethyl chloroformate (0.006 mL, 0.065 mmol) was added dropwise. The reaction mixture was stirred at the same temperature for 1 h and the solid was filtered off. The filtrate was added to a solution of hydroxylamine hydrochloride (6 mg, 0.089 mmol) and Et₃N (0.012 mL, 0.089 mmol) in DMF (0.5 mL) at 0° C. The reaction mixture was stirred at 25° C. for 48 h and the solvent was removed under reduced pressure. The residue was extracted with EtOAc, washed with water, dried over anhydrous Na₂SO₄, and concentrated in vacuo. The residue was purified by column chromatography (silica gel, CH₂Cl₂/MeOH, 10/1) to afford Compound 16 (11 mg, 39%): ¹H NMR (400 MHz, (CD₃)₂CO) δ 10.20 (s, 1H), 8.16 (d, J=8.0 Hz, 2H), 7.84 (d, J=8.0 Hz, 2H), 7.42 (m, 1H), 7.22 (m, 2H), 7.11 (m, 1H), 3.39 (m, 4H), 3.16 (m, 4H); HRMS (ESI) m/z 473.1102 [(M+H)⁺, C₁₉H₁₉F₃N₄O₅S requires 473.1101].

### Example 13M

3-((4-(3-(Trifluoromethyl)phenyl)piperazin-1-yl)sulfonyl)benzoic acid (Compound 19), the structure of which is shown below, was synthesized according to the following procedure.

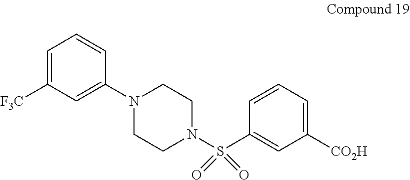 [SMILES: O=C(O)c1cccc(S(=O)(=O)N2CCN(c3cccc(C(F)(F)F)c3)CC2)c1]

Et₃N (0.022 mL, 0.16 mmol) was added to a solution of 1-(3-(trifluoromethyl)phenyl)piperazine (30 mg, 0.13 mmol) in 1,4-dioxane (2.6 mL, 0.05 M). The reaction mixture was heated until the temperature reached 60° C. 3-Carboxybenzene sulfonyl chloride (29 mg, 0.13 mmol) in 1,4-dioxane (1 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄ and concentrated in vacuo. The residue was purified by column chromatography (silica gel, hexanes/EtOAc, 3/1) to afford Compound 19 (22 mg, 40%): ¹H NMR (400 MHz, CD₃OD) δ 8.36 (s, 1H), 8.21 (d, J=7.6 Hz, 1H), 7.83 (d, J=8.0 Hz, 1H), 7.61 (t, J=7.6 Hz, 1H), 7.35 (t, J=7.6 Hz, 1H), 7.13 (m, 2H), 7.06 (d, J=7.6 Hz, 1H), 3.28 (m, 6H), 3.15 (m, 4H); HRMS (ESI) m/z 415.0938 [(M+H)⁺, C₁₈H₁₇F₃N₂O₄S requires 415.0934].

### Example 13N

2-((4-(3-(Trifluoromethyl)phenyl)piperazin-1-yl)sulfonyl)benzoic acid (Compound 20), the structure of which is shown below, was synthesized according to the following procedure.

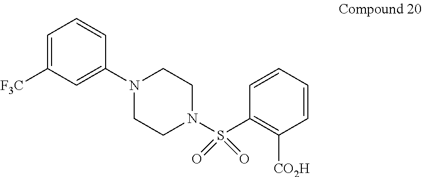 [SMILES: O=C(O)c1ccccc1S(=O)(=O)N1CCN(c2cccc(C(F)(F)F)c2)CC1]

Et₃N (0.022 mL, 0.16 mmol) was added to a solution of 1-(3-(trifluoromethyl)phenyl)piperazine (30 mg, 0.13 mmol) in 1,4-dioxane (2.6 mL, 0.05 M). The reaction mixture was heated until the temperature reached 60° C. 3-(Carbomethoxy)benzene sulfonyl chloride (31 mg, 0.13 mmol) in 1,4-dioxane (1 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄ and concentrated in vacuo. The residue was purified by column chromatography (silica gel, hexanes/EtOAc, 1/2) to afford an intermediate (27 mg, 49%): ¹H NMR (400 MHz, CDCl₃) δ 7.85 (d, J=7.2 Hz, 1H), 7.65-7.58 (m, 2H), 7.51 (d, J=7.2 Hz, 1H), 7.34 (t, J=8.0 Hz, 1H), 7.10 (d, J=7.6 Hz, 1H), 7.06 (s, 1H), 7.02 (d, J=8.0 Hz, 1H), 3.95 (s, 3H), 3.37 (m, 4H), 3.27 (m, 4H). To a solution of the intermediate (27 mg, 0.064 mmol) in MeOH (1 mL)/THF (0.5 mL), a solution of LiOH·H₂O (42 mg, 1.0 mmol) in H₂O (0.5 mL) was added. The reaction mixture was refluxed for 3 h and concentrated in vacuo. Water was added and the aqueous layer was washed with EtOAc, and then acidified to pH 5 with citric acid. The aqueous layer was extracted with EtOAc and the combined organic layers were dried over anhydrous Na₂SO₄, and concentrated in vacuo. The residue was purified by column chromatography (silica gel, hexanes/EtOAc, 1/1) to afford Comound 20 as a white powder (21 mg, 81%): ¹H NMR (400 MHz, CDCl₃) δ 7.92 (d, J=7.6 Hz, 1H), 7.65 (m, 3H), 7.35 (t, J=8.0 Hz, 1H), 7.12 (d, J=7.6 Hz, 1H), 7.08 (s, 1H), 7.03 (d, J=8.0 Hz, 1H), 3.42 (m, 4H), 3.28 (m, 4H); HRMS (ESI) m/z 415.0937 [(M+H)⁺, C₁₈H₁₇F₃N₂O₄S requires 415.0934].

### Example 13O

4-(5-((4-(3-Bromophenyl)piperazin-1-yl)sulfonyl)indolin-1-yl)-4-oxobutanoic acid (Compound 7), the structure of which is shown below, was synthesized according to the following procedure.

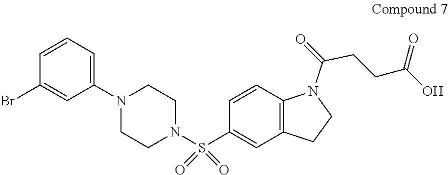 [SMILES: O=C(O)CCC(=O)N1CCc2cc(S(=O)(=O)N3CCN(c4cccc(Br)c4)CC3)ccc21]

To a cooled (0° C.) solution of 1-(3-bromophenyl)piperazine (29 mg, 0.12 mmol) and pyridine (0.2 mL) in CH₂Cl₂ (10 mL), the sulfonyl chloride methyl 4-(5-(chlorosulfonyl)indolin-1-yl)-4-oxobutanoate (40 mg, 0.12 mmol) was added. The reaction mixture was warmed to 25° C. and stirred for 14 h. Water and 37% HCl (0.1 mL) were added to the mixture. The organic layer was separated, washed with brine, dried over Na₂SO₄, and concentrated in vacuo. The residue was triturated with MeOH to afford Compound 6 (45 mg, 70%): ¹H NMR (400 MHz, CDCl₃) δ 8.32 (d, J=8.4 Hz, 1H), 7.60 (dd, J=8.4, 2.0 Hz, 1H), 7.56 (d, J=2.0 Hz, 1H), 7.08 (t, J=8.4 Hz, 1H), 6.97 (m, 2H), 6.76 (m, 1H), 4.20 (t, J=8.4 Hz, 2H), 3.71 (s, 3H), 3.28 (t, J=8.4 Hz, 2H), 3.23 (m, 4H), 3.12 (m, 4H), 2.76 (s, 4H); HRMS (ESI) m/z 536.0852 [(M+H)⁺, C₂₃H₂₆BrN₃O₅S requires 536.0849]. To a solution of Compound 6 (12 mg, 0.022 mmol) in THF (1 mL) was added MeOH (0.5 mL) and then 1 N NaOH (0.22 mL, 0.22 mmol). The reaction mixture was stirred at 25° C. for 1 h. The pH was adjusted to 4 with 1 N HCl, and the reaction mixture was extracted with EtOAc. The combined organic layers were washed with brine, dried over anhydrous Na₂SO₄, and concentrated in vacuo to afford Compound 7 (10 mg, 91%): ¹H NMR (400 MHz, CDCl₃) δ 8.21 (d, J=8.4 Hz, 1H), 7.60 (m, 2H), 7.13 (m, 1H), 7.05 (m, 1H), 6.94-6.88 (m, 2H), 4.19 (m, 2H), 3.35-3.29 (m, 6H), 3.24 (m, 4H), 2.96 (m, 4H); HRMS (ESI) m/z 522.0689 [(M+H)⁺, C₂₂H₂₄BrN₃O₅S requires 522.0698].

### Example 13P

1-(5-((4-(3-(Trifluoromethyl)phenyl)-1,4-diazepan-1-yl)sulfonyl)indolin-1-yl)ethan-1-one (Compound 17), the structure of which is shown below, was synthesized according to the following procedure.

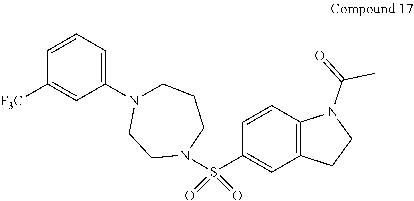 [SMILES: CC(=O)N1CCc2cc(S(=O)(=O)N3CCCN(c4cccc(C(F)(F)F)c4)CC3)ccc21]

TFA (1 mL) was added dropwise to a cooled (0° C.) solution of tert-butyl 4-(3-(trifluoromethyl)phenyl)-1,4-diazepane-1-carboxylat (76 mg, 0.22 mmol) in dry CH₂Cl₂ (5 mL). After stirring 1 h at 25° C., the solvent was removed under reduced pressure. The residue was dissolved in 1,4-dioxane (4 mL, 0.055 M) and Et₃N (0.04 mL, 0.26 mmol) was added. The reaction mixture was heated until the temperature reached 60° C. 1-Acetyl-5-indolinesulfonyl chloride (57 mg, 0.22 mmol) in 1,4-dioxane (2 mL) was added dropwise to the reaction mixture, which was stirred at 60° C. for 3 h, and then maintained at 25° C. overnight. Water was added and the aqueous layer was extracted with EtOAc. The combined organic layers were dried over anhydrous Na₂SO₄, and concentrated in vacuo. The residue was purified by column chromatography (silica gel, hexanes/EtOAc, 1/2) to afford Compound 17 as a white powder (93 mg, 92%): ¹H NMR (400 MHz, CDCl₃) δ 8.23 (d, J=8.0 Hz, 1H), 7.59 (d, J=8.0 Hz, 1H), 7.51 (s, 1H), 7.26 (m, 2H), 6.90 (d, J=7.6 Hz, 1H), 6.78 (m, 2H), 4.10 (t, J=8.4 Hz, 2H), 3.65 (m, 4H), 3.44 (m, 2H), 3.17 (m, 4H), 2.24 (s, 3H), 2.04 (t, J=6.0 Hz, 2H); HRMS (ESI) m/z 468.1567 [(M+H)⁺, C₂₂H₂₄F₃N₃O₃S requires 468.1563].

### Example 13Q

1-Tosyl-5-(3-(trifluoromethyl)phenyl)-1,5-diazocane, the structure of which is shown below, was synthesized according to the following procedure.

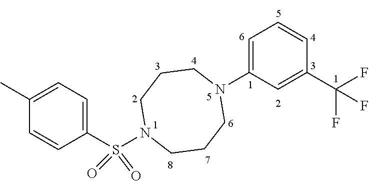 [SMILES: Cc1ccc(S(=O)(=O)N2CCCN(c3cccc(C(F)(F)F)c3)CCC2)cc1]

A mixture of 3-iodobenzotrifluoride (0.04 mL, 0.27 mmol), 1-tosyl-1,5-diazocane (110 mg, 0.41 mmol), CuI (3 mg, 0.014 mmol), and Cs₂CO₃ (178 mg, 0.55 mmol) in DMF (2 mL) was degassed for 5 min and then 2-isobutyrylcyclohexanone (0.009 mL, 0.055 mmol) was added. The resulting mixture was stirred at 70° C. for 16 h. The product was extracted with EtOAc and washed with water, 1% HCl, and brine. The organic layer was dried over anhydrous Na₂SO₄ and concentrated in vacuo. The residue was purified by column chromatography (silica gel, Hexane/EtOAc, 5/1) to afford 1-tosyl-5-(3-(trifluoromethyl)phenyl)-1,5-diazocane (33 mg, 30%): ¹H NMR (400 MHz, CDCl₃) δ 7.65 (d, J=8.0 Hz, 2H), 7.27 (m, 3H), 6.88 (d, J=7.2 Hz, 1H), 6.74 (m, 2H), 3.64 (m, 4H), 3.13 (m, 4H), 2.40 (s, 3H), 1.96 (m, 4H); HRMS (ESI) m/z 413.1505 [(M+H)⁺, C₂₀H₂₃F₃N₂O₂S requires 413.1505].

### Example 13R

1-(5-((5-(3-(Trifluoromethyl)phenyl)-1,5-diazocan-1-yl)sulfonyl)indolin-1-yl)ethan-1-one (Compound 18), the structure of which is shown below, was synthesized according to the following procedure.

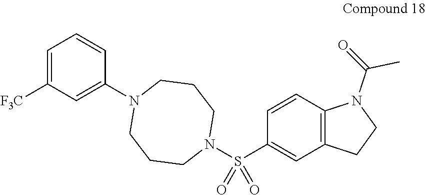 [SMILES: CC(=O)N1CCc2cc(S(=O)(=O)N3CCCN(c4cccc(C(F)(F)F)c4)CCC3)ccc21]

Phenol (30 mg, 0.32 mmol) was added to a solution of 1-tosyl-5-(3-(trifluoromethyl)phenyl)-1,5-diazocane (33 mg, 0.08 mmol) in HBr (33% in HOAc, 1 mL). The reaction mixture was heated at 90° C. for 5 h and concentrated in vacuo. The residue was dissolved in water and extracted with EtOAc. The aqueous layer was adjusted to pH 12 by addition of 1 N NaOH solution and extracted with CHCl₃. The combined organic layers were washed with brine, dried over anhydrous Na₂SO₄, and concentrated in vacuo. The residue was purified by column chromatography (silica gel, CH₂Cl₂/MeOH/NH₄OH, 8/1/0.1) to afford the 1,5-diazacyclooctane (8 mg, 38%): ¹H NMR (400 MHz, CDCl₃) δ 7.30 (t, J=8.0 Hz, 1H), 6.90-6.79 (m, 3H), 3.56 (m, 4H), 2.92 (m, 4H), 2.78 (s, 1H), 1.85 (m, 4H); HRMS (ESI) m/z 259.1417 [(M+H)⁺, C₁₃H₁₇F₃N₂ requires 259.1417]. Et₃N (0.004 mL, 0.028 mmol) was added to a solution of the 1,5-diazacyclooctane (6 mg, 0.023 mmol) in 1,4-dioxane (1 mL, 0.023 M). The reaction mixture was heated until the temperature reached 60° C. 1-Acetyl-5-indolinesulfonyl chloride (6 mg, 0.023 mmol) in 1,4-dioxane (0.5 mL) was added dropwise to the reaction mixture, which was stirred at 60° C. for 3 h, and then maintained at 25° C. overnight. Water was added and the aqueous layer was extracted with EtOAc. The combined organic layers were dried over anhydrous Na₂SO₄ and concentrated in vacuo. The residue was purified by column chromatography (silica gel, hexanes/EtOAc, 1/2) to afford Compound 18 (4 mg, 36%): ¹H NMR (400 MHz, CDCl₃) δ 8.27 (d, J=8.0 Hz, 1H), 7.62 (d, J=8.4 Hz, 1H), 7.56 (s, 1H), 7.29 (t, J=7.2 Hz, 1H), 6.88 (d, J=7.2 Hz, 1H), 6.74 (m, 2H), 4.12 (t, J=8.8 Hz, 2H), 3.64 (m, 4H), 3.21 (t, J=8.8 Hz, 2H), 3.12 (m, 4H), 2.25 (s, 3H), 1.97 (m, 4H); HRMS (ESI) m/z 482.1724 [(M+H)⁺, C₂₃H₂₆F₃N₃O₃S requires 482.1720].

### Example 13S

1-Acetyl-N-(2-((3-(trifluoromethyl)phenyl)amino)ethyl)indoline-5-sulfonamide (Compound 13), the structure of which is shown below, was synthesized according to the following procedure.

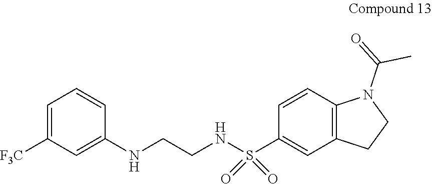 [SMILES: CC(=O)N1CCc2cc(S(=O)(=O)NCCNc3cccc(C(F)(F)F)c3)ccc21]

Et₃N (0.015 mL, 0.11 mmol) was added to a solution of N¹-(3-(trifluoromethyl)phenyl)ethane-1,2-diamine (19 mg, 0.093 mmol) in 1,4-dioxane (1 mL, 0.093 M). The reaction mixture was heated until the temperature reached 60° C. 1-Acetyl-5-indolinesulfonyl chloride (24 mg, 0.093 mmol) in 1,4-dioxane (0.5 mL) was added dropwise to the reaction mixture, which was stirred at 60° C. for 3 h, and then at 25° C. overnight. Water was added and the aqueous layer was extracted with EtOAc. The combined organic layers were dried over anhydrous Na₂SO₄ and concentrated in vacuo. The residue was purified by column chromatography (silica gel, hexanes/EtOAc, 1/2) to afford Compound 13 as a white powder (32 mg, 80%): ¹H NMR (400 MHz, CD₃CN) δ 8.16 (d, J=7.2 Hz, 1H), 7.60 (m, 2H), 7.24 (t, J=8.0 Hz, 1H), 6.89 (d, J=8.0 Hz, 1H), 6.75 (m, 2H), 5.67 (m, 1H), 4.10 (t, J=8.4 Hz, 2H), 3.18 (m, 4H), 3.02 (m, 2H), 2.16 (s, 3H); HRMS (ESI) m/z 428.1254 [(M+H)⁺, C₁₉H₂₀F₃N₃O₃S requires 428.1250].

### Example 13T

1-(5-((4-(3-(Trifluoromethyl)benzyl)piperazin-1-yl)sulfonyl)indolin-1-yl)ethan-1-one (Compound 21), the structure of which is shown below, was synthesized according to the following procedure.

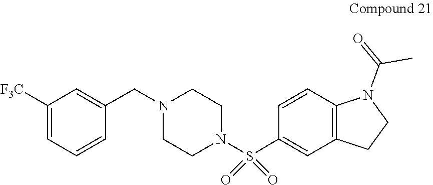 [SMILES: CC(=O)N1CCc2cc(S(=O)(=O)N3CCN(Cc4cccc(C(F)(F)F)c4)CC3)ccc21]

Et₃N (0.018 mL, 0.13 mmol) was added to a solution of 1-(3-(trifluoromethyl)benzyl)piperazine (26 mg, 0.11 mmol) in 1,4-dioxane (1 mL, 0.11 M). The reaction mixture was heated until the temperature reached 60° C. 1-Acetyl-5-indolinesulfonyl chloride (29 mg, 0.11 mmol) in 1,4-dioxane (0.5 mL) was added dropwise to the reaction mixture, which was stirred at 60° C. for 3 h, and then maintained at 25° C. overnight. Water was added and the aqueous layer was extracted with EtOAc. The combined organic layers were dried over anhydrous Na₂SO₄ and concentrated in vacuo. The residue was purified by column chromatography (silica gel, hexanes/EtOAc, 1/5) to afford Compound 21 (37 mg, 74%): ¹H NMR (400 MHz, CDCl₃) δ 8.31 (d, J=8.4 Hz, 1H), 7.57 (d, J=8.4 Hz, 1H), 7.48 (m, 3H), 7.43-7.36 (m, 2H), 4.14 (t, J=8.4 Hz, 2H), 3.52 (s, 2H), 3.25 (t, J=8.4 Hz, 2H), 3.00 (m, 4H), 2.51 (m, 4H), 2.25 (s, 3H); HRMS (ESI) m/z 468.1570 [(M+H)⁺, C₂₂H₂₄F₃N₃O₃S requires 468.1563].

### Example 13U

tert-Butyl-(E)-4-(3-(5-((4-methoxybenzyl)oxy)-4-oxo-4H-pyran-2-yl)allyl)piperazine-1-carboxylate, the structure of which is shown below, was synthesized according to the following procedure.

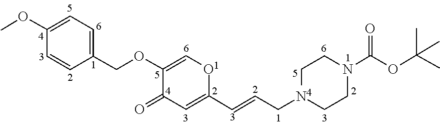

To a cooled (0° C.) solution of the known phosphate diethyl ((5-((4-methoxybenzyl)oxy)-4-oxo-4H-pyran-2-yl)methyl)phosphonate (65 mg, 0.17 mmol) and NaH (6.8 mg, 0.17 mmol) in dry THF (1.5 mL), a solution of the known aldehyde tert-butyl 4-(2-oxoethyl)piperazine-1-carboxylate (39 mg, 0.17 mmol) in THF (1.5 mL) was added dropwise. The reaction mixture was stirred at 25° C. for 18 h and diluted with CH₂Cl₂. The mixture was washed with water and brine, dried over anhydrous Na₂SO₄, and concentrated in vacuo. The residue was purified by column chromatography (silica gel, EtOAc/MeOH, 10/1) to afford tert-butyl-(E)-4-(3-(5-((4-methoxybenzyl)oxy)-4-oxo-4H-pyran-2-yl)allyl)piperazine-1-carboxylate as a yellow oil (53 mg, 68%): ¹H NMR (400 MHz, CDCl₃) δ 7.47 (s, 1H), 7.31 (d, J=8.4 Hz, 2H), 6.88 (d, J=8.4 Hz, 2H), 6.58-6.51 (m, 1H), 6.24 (s, 1H), 6.19 (d, J=15.6 Hz, 1H), 5.01 (s, 2H), 3.79 (s, 3H), 3.45 (m, 4H), 3.17 (m, 2H), 2.42 (m, 4H), 1.44 (s, 9H).

### Example 13V

5-((4-Methoxybenzyl)oxy)-2-(3-(piperazin-1-yl)propyl)-4H-pyran-4-one, the structure of which is shown below, was synthesized according to the following procedure.

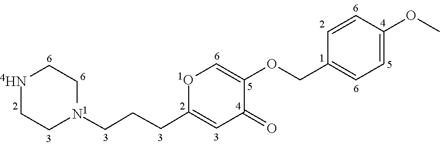

To a solution of tert-butyl-(E)-4-(3-(5-((4-methoxybenzyl)oxy)-4-oxo-4H-pyran-2-yl)allyl)piperazine-1-carboxylate (52 mg, 0.11 mmol) in EtOAc (3 mL) and EtOH (1 mL), Pd/C (10.4 mg, 0.2 wt %) was added. The reaction mixture was stirred at 25° C. for 2 h under H₂ gas and filtered through a Celite pad. The filtrate was concentrated in vacuo and the residue was purified by column chromatography (silica gel, EtOAc/MeOH, 10/1) to afford the alkane as a yellow oil (34 mg, 67%): ¹H NMR (400 MHz, CDCl₃) δ 7.47 (s, 1H), 7.31 (d, J=8.4 Hz, 2H), 6.88 (d, J=8.4 Hz, 2H), 6.22 (s, 1H), 5.00 (s, 2H), 3.81 (s, 3H), 3.43 (m, 4H), 2.54 (t, J=7.2 Hz, 2H), 2.36 (m, 6H), 1.81 (m, 2H), 1.46 (s, 9H). To a cooled (0° C.) solution of the alkane (22 mg, 0.048 mmol) in CH₂Cl₂ (1.5 mL), a solution of TMSOTf (0.026 mL, 0.144 mmol) in CH₂Cl₂ (0.5 mL) was added dropwise. The reaction mixture was stirred at 0° C. for 5 min quenched with sat. NaHCO₃, was extracted with CH₂Cl₂. The combined organic layers were dried over anhydrous Na₂SO₄, and concentrated in vacuo to afford 5-((4-methoxybenzyl)oxy)-2-(3-(piperazin-1-yl)propyl)-4H-pyran-4-one (13 mg, 76%). The crude residue was used in the next step without further purification.

### Example 13W

2-(3-(4-((1-Acetylindolin-5-yl)sulfonyl)piperazin-1-yl)propyl)-5-hydroxy-4H-pyran-4-one (Compound 4), the structure of which is shown below, was synthesized according to the following procedure.

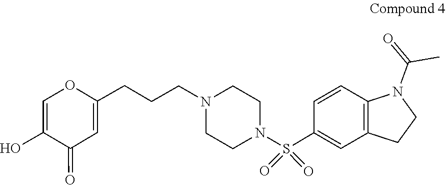 [SMILES: CC(=O)N1CCc2cc(S(=O)(=O)N3CCN(CCCc4cc(=O)c(O)co4)CC3)ccc21]

Et₃N (0.006 mL, 0.043 mmol) was added to a solution of 5-((4-Methoxybenzyl)oxy)-2-(3-(piperazin-1-yl)propyl)-4H-pyran-4-one (13 mg, 0.036 mmol) in 1,4-dioxane (1 mL). The reaction mixture was heated until the temperature reached 60° C. 1-Acetyl-5-indolinesulfonyl chloride (9 mg, 0.036 mmol) in 1,4-dioxane (1 mL) was added dropwise to the reaction mixture, which was stirred at 60° C. for 3 h, and then maintained at 25° C. overnight. Water was added and the aqueous layer was extracted with EtOAc. The combined organic layers were dried over anhydrous Na₂SO₄, and concentrated in vacuo. The residue was purified by column chromatography (silica gel, EtOAc/MeOH, 7/1) to afford an intermediate (15.6 mg, 74%). TFA (0.3 mL) was added to a cooled (0° C.) solution of the intermediate (12 mg, 0.021 mmol) in CH₂Cl₂ (1.5 mL). The reaction mixture was stirred at 25° C. for 30 min and concentrated in vacuo. The residue was dissolved in EtOAc and washed with sat. NaHCO₃, brine, dried over anhydrous Na₂SO₄, and concentrated in vacuo. The residue was purified by column chromatography (silica gel, EtOAc/MeOH, 7/1) to afford Compound 4 (9 mg, 94%): ¹H NMR (400 MHz, CD₃OD) δ 8.30 (d, J=8.4 Hz, 1H), 7.93 (s, 1H), 7.65 (m, 2H), 6.32 (s, 1H), 4.23 (t, J=8.0 Hz, 2H), 3.33 (m, 4H), 3.20 (m, 4H), 2.68 (t, J=8.0 Hz, 2H), 2.28 (s, 3H), 2.06 (m, 2H), 1.30 (m, 2H); HRMS (ESI) m/z 462.1694 [(M+H)⁺, C₂₂H₂₇N₃O₆S requires 462.1693].

### Example 13X

1-(5-(4-(3-(Trifluoromethyl)phenyl)piperazine-1-carbonyl)indolin-1-yl)ethan-1-one (Compound 43), the structure of which is shown below, was synthesized according to the following procedure.

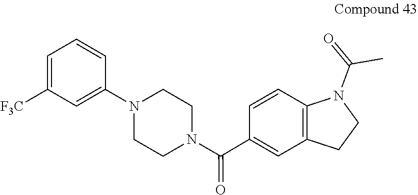 [SMILES: CC(=O)N1CCc2cc(C(=O)N3CCN(c4cccc(C(F)(F)F)c4)CC3)ccc21]

To a cooled (0° C.) solution of 1-acetylindoline-5-carboxylic acid (35 mg, 0.17 mmol), EDC (38 mg, 0.2 mmol), and HOBt (27 mg, 0.2 mmol) in CH₂Cl₂ (2 mL), DIPEA (0.11 mL, 0.61 mmol) and then 1-(3-(trifluoromethyl)phenyl)piperazine (39 mg, 0.17 mmol) were added. The reaction mixture was stirred at 25° C. for 16 h and water was added and separated. The aqueous layer was extracted with CH₂Cl₂. The combined organic layers were subsequently washed with 1 N NaOH, 1 N HCl, and brine, dried over anhydrous Na₂SO₄, and concentrated in vacuo. The residue was purified by column chromatography (silica gel, EtOAc) to afford 1-(5-(4-(3-(trifluoromethyl)phenyl)piperazine-1-carbonyl)indolin-1-yl)ethan-1-one (52 mg, 73%): ¹H NMR (400 MHz, CDCl₃) δ 8.22 (d, J=8.4 Hz, 1H), 7.39-7.33 (m, 2H), 7.26 (m, 1H), 7.14-7.01 (m, 3H), 4.10 (t, J=8.0 Hz, 2H), 3.79 (m, 4H), 3.23 (m, 4H), 2.25 (s, 3H); HRMS (ESI) m/z 418.1741 [(M+H)⁺, C₂₂H₂₂F₃N₃O₂ requires 418.1737].

### Example 14: Synthesis of Analogs Toward E. coli LpxH

A series of LpxH targeting analogs were synthesized in order to establish a preliminary SAR of Compound 1. The structure of Compound 1 is highly modular: it contains a trifluoromethyl substituted phenyl ring and an N-acetyl indoline group that are connected by a central sulfonyl piperazine linker.

Compound 1 phenyl group analogs were prepared by replacing the trifluoromethyl group with various functional groups, including halogen, alkyl, and carboxylate groups to assess the importance of the trifluoromethyl group of AZ1 in LpxH inhibition. Commercially available m-substituted phenyl piperazines-namely, 1-(3-bromophenyl)piperazine (A), 1-phenylpiperazine (B), 1-(3-ethylphenyl)piperazine (C), 1-(3-isopropylphenyl)piperazine (D), and 1-([1,1′-biphenyl]-3-yl)piperazine (E)—were coupled to the commercially available 1-acetyl-5-indolinesulfonyl chloride pursuant to reaction scheme 4A, provided below. The coupling reaction proceeded smoothly in the presence of Et₃N to afford the following compounds: Compound 5, 09, 24, 26, and 25) in 43-74% yield.

Next, treatment of 3-(tert-butyldimethylsilyloxy)aniline with 2,2′-dichlorodiethylamine hydrochloride gave the desired 1-(3-((tert-butyldimethylsilyl)oxy)phenyl)piperazine (see Reaction Scheme 4B, provided below). Coupling of 1-(3-((tert-butyldimethylsilyl)oxy)phenyl)piperazine and 1-acetylindoline-5-sulfonyl chloride followed by final TBS deprotection gave the phenol analog 2,2′-dichlorodiethylamine hydrochloride in 75% yield. Carboxylate analogs Compound 10 and Compound 11 were prepared from 3-carbomethoxyaniline in a similar manner (see Scheme 4B).

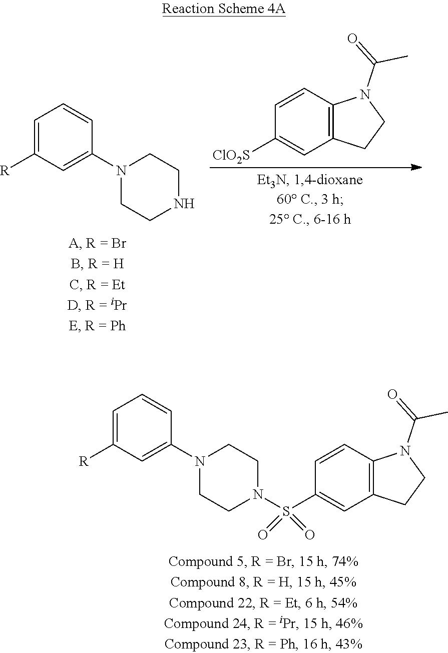

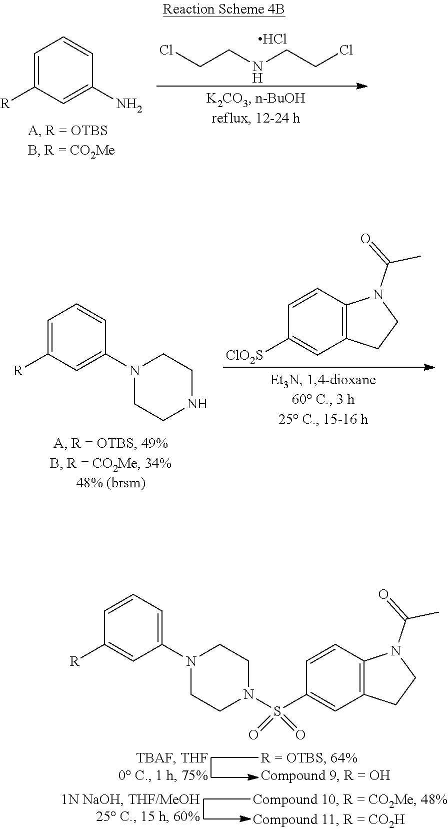

A series of N-acetyl indoline group analogs were prepared to assess the effect of the N-acetyl and indoline groups of Compound 1 in LpxH inhibition. First, N-acetylsulfanil analog Compound 14 was prepared by replacing the indoline ring of Compound 1 with aniline, but keeping the N-acetyl group (see Reaction Scheme 5A). The N-acetyl group was also replaced with extended carbonyl chains such as 1,3-diketo hydroxamic acid Compound 15 in order to assess the effect of chain elongation and potential metal binding of the hydoxamic acid group in Compound 15 on activity. The synthesis of Compound 15 began with coupling of 1-(3-(trifluoromethyl)phenyl)piperazine to commercially available 4-acetamidobenzenesulfonyl chloride. Then, Ac deprotection of the N-acetylsulfanil analog Compound 14 followed by coupling of the resulting aniline with mono-ethyl malonate gave the oxoacetate ethyl-3-oxo-3-((4-((4-(3-(Trifluoromethyl)phenyl)piperazin-1-yl)sulfonyl)phenyl)amino)propanoate. Compound ethyl-3-oxo-3-((4-((4-(3-(Trifluoromethyl)phenyl)piperazin-1-yl)sulfonyl)phenyl)amino)propanoate was subsequently hydrolyzed by LIOH to afford the corresponding carboxylic acid. PyBOP-mediated coupling with NH₂OTBS successfully provided the desired hydroxamic acid analog Compound 15, but in low yield (35%) (see Reaction Scheme 5A). In a similar manner, the synthesis of the phenyloxalamide analog (Compound 16) began with coupling of 4-((4-(3-(trifluoromethyl)phenyl)piperazin-1-yl)sulfonyl)aniline with methyl chlorooxoacetate to give the oxoacetate labeled “15” in Reaction Scheme 5B, provided below. Basic hydrolysis of 15 by LiOH followed by installation of a hydroxamic acid group completed the synthesis of Compound 16.

To mimic the hydrogen bonding capability of the N-acetyl group, 1-(3-(trifluoromethyl)phenyl)piperazine was coupled with 3-carboxybenezne sulfonyl chloride and 3-(carbomethoxy)benzene sulfonyl chloride to provide Compound 19 and Compound 20, respectively (see Reaction Scheme 5C).

Indoline 5-oxo-butanoic acid analogs, Compound 6 and Compound 7, were prepared by modifying both the trifluoromethyl and N-acetyl groups of Compound 1 as illustrated in Reaction Scheme 5D. Coupling of the known sulfonyl chloride labelled 21¹⁵ in Reaction Scheme 5D with 1-(3-bromophenyl)piperazine provided the desired Compound 6, which was subsequently hydrolyzed to afford Compound 7.

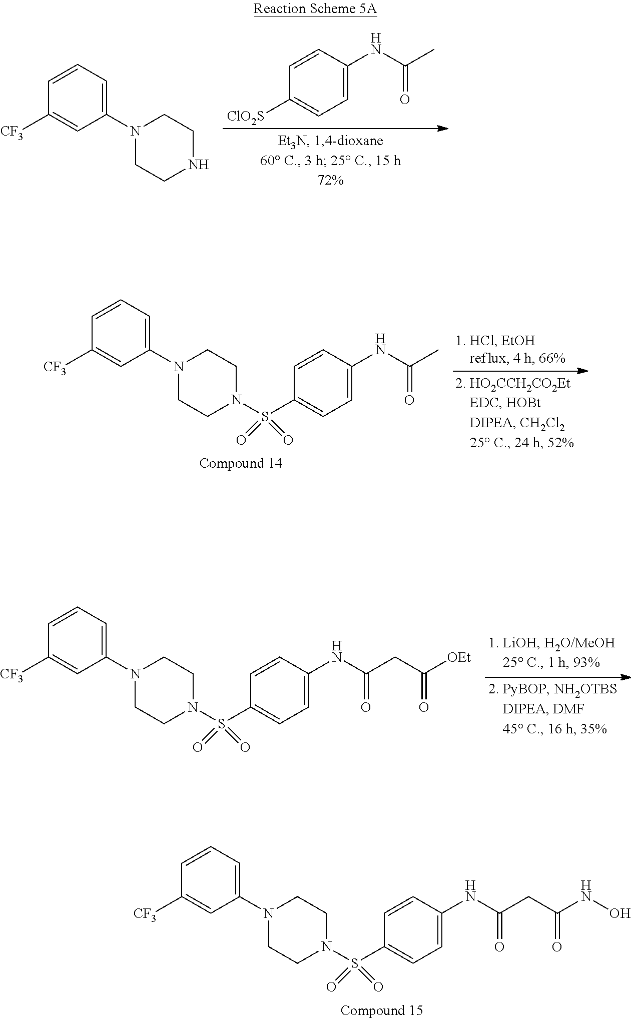 [SMILES: FC(F)(F)c1cccc(N2CCNCC2)c1 ; CC(=O)Nc1ccc(S(=O)(=O)Cl)cc1 ; CC(=O)Nc1ccc(S(=O)(=O)N2CCN(c3cccc(C(F)(F)F)c3)CC2)cc1 ; CCOC(=O)CC(=O)Nc1ccc(S(=O)(=O)N2CCN(c3cccc(C(F)(F)F)c3)CC2)cc1 ; O=C(CC(=O)Nc1ccc(S(=O)(=O)N2CCN(c3cccc(C(F)(F)F)c3)CC2)cc1)NO]

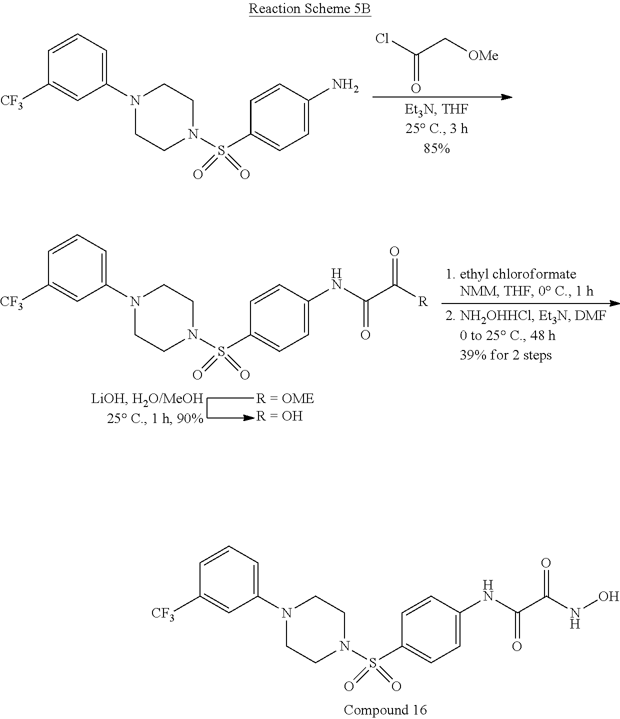

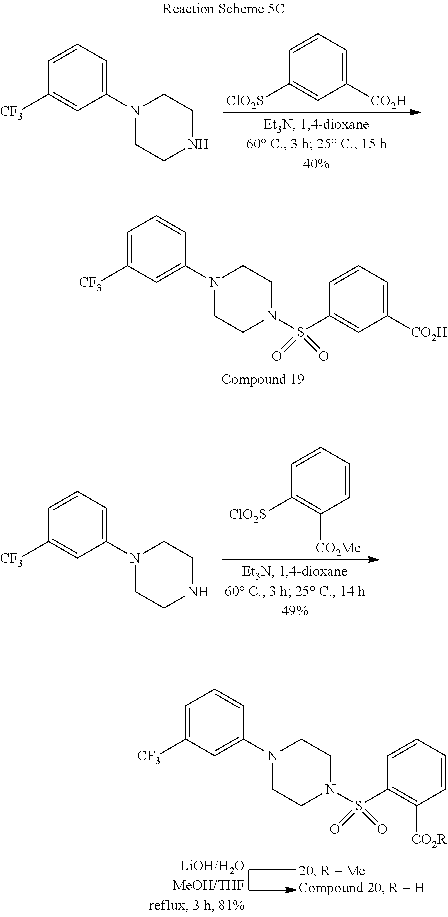

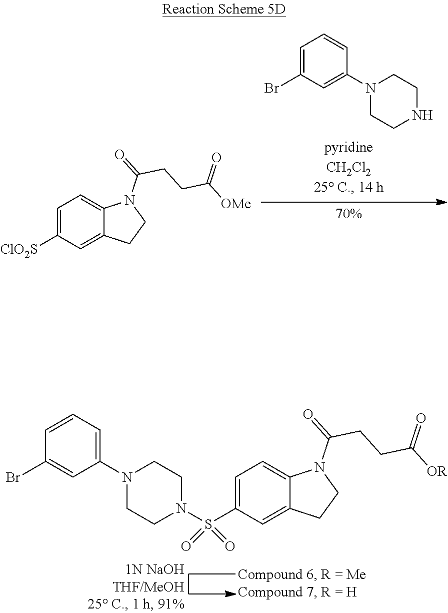

In order to assess whether sulfonyl piperazine linker of Compound 1 provides the optimal distance between the trifluoromethylphenyl ring and the N-acetyl indoline group and whether it interacts with LpxH, several sulfonamide linker analogs were prepared. First, two larger core ring analogs, namely Compound 17 and Compound 18, prepared by replacing the piperazine group of Compound 1 with 1,4-diazacycloheptane and 1,5-diazacyclooctane, respectively (see Reaction Scheme 6).

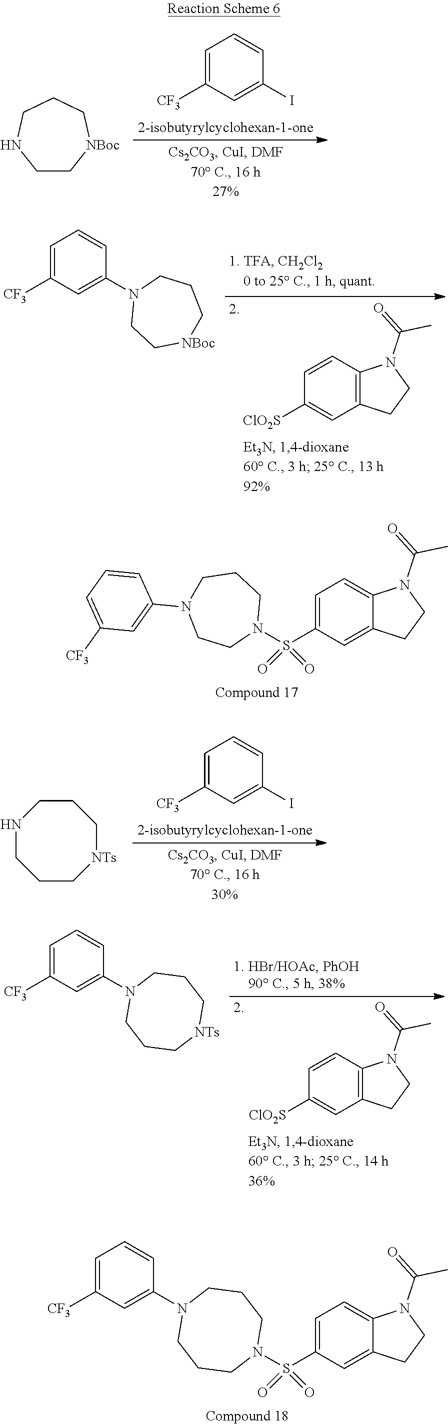

To evaluate the effect of structural rigidity, the flexible acyclic linker analog Compound 13 was prepared and assessed. The known N¹-(3-(trifluoromethyl)phenyl)ethane-1,2-diamine was treated with 1-acetyl-5-indolinesulfonyl chloride to afford the desired acyclic analog Compound 13 in 80% yield (see Reaction Scheme 7A). The one-carbon homologated analog Compound 21 was prepared in a similar manner. To take advantage of the potential binding of the kojic acid group to the di-Mn²⁺ cluster of the LpxH active site, the Compound 1 kojic acid analog Compound 4 was synthesized from the phosphonate 31¹⁸ and aldehyde 32¹⁹ (see Reaction Scheme 7B). To define the role of the sulfonamide group of Compound 1, standard EDC-HOBt amide coupling condition was used and prepared the corresponding amide analog Compound 43 (see Reaction Scheme 7C).

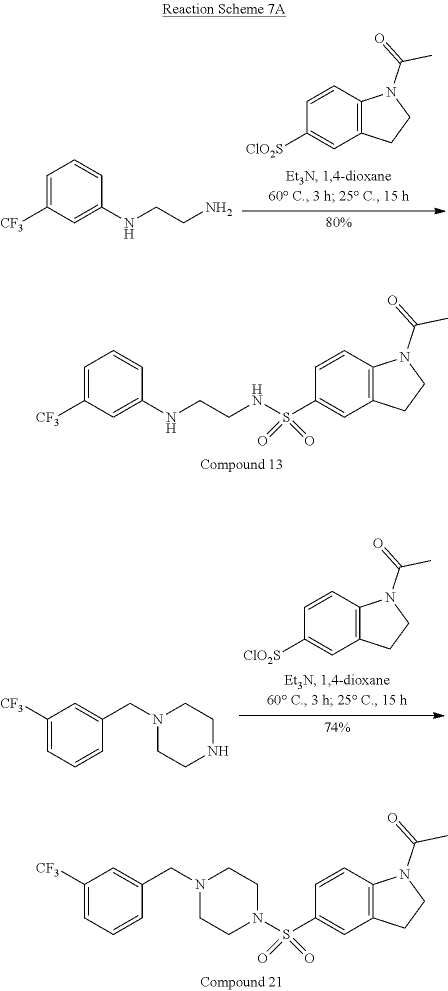 [SMILES: CC(=O)N1CCc2cc(S(=O)(=O)Cl)ccc21 ; NCCNc1cccc(C(F)(F)F)c1 ; CC(=O)N1CCc2cc(S(=O)(=O)NCCNc3cccc(C(F)(F)F)c3)ccc21 ; CC(=O)N1CCc2cc(S(=O)(=O)Cl)ccc21 ; FC(F)(F)c1cccc(CN2CCNCC2)c1 ; CC(=O)N1CCc2cc(S(=O)(=O)N3CCN(Cc4cccc(C(F)(F)F)c4)CC3)ccc21]

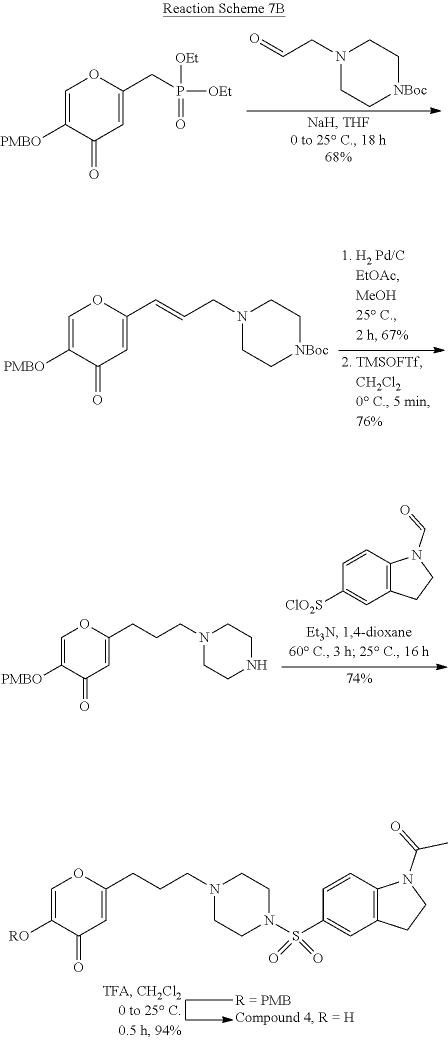

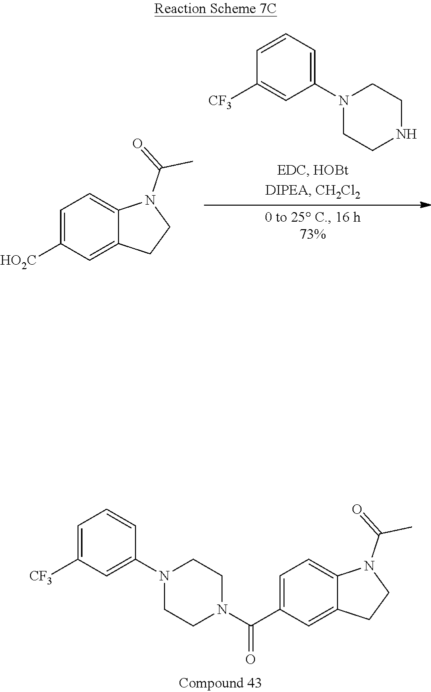 [SMILES: CC(=O)N1CCc2cc(C(=O)O)ccc21 ; FC(F)(F)c1cccc(N2CCNCC2)c1 ; CC(=O)N1CCc2cc(C(=O)N3CCN(c4cccc(C(F)(F)F)c4)CC3)ccc21]

### Example 15: Analysis of SAR of LpxH Targeting Analog Compound

Among the tested LpxH targeting analogs, the m-bromo phenyl piperazine analog Compound 5 showed the strongest inhibition of LpxH activity (see Table 4). Compound 5 inhibited 74% of LpxH activity at 1 μM. Several other LpxH targeting analogs-namely, Compound 6, Compound 7, and Compound 23—also showed a useful level of LpxH inhibitory activity. The LpxH activity assay data provided several valuable insights into the SAR.

Among the synthesized LpxH targeting phenyl group analogs, analogs with a hydrophobic substituent on the trifluoromethyl phenyl ring (e.g., Compound 5 and Compound 23) were active, but analogs with a polar functional group, such as the phenol analog Compound 9 and the benzoic acid analog Compound 11, were inactive. Without being limited to any specific theory, it is believed that a bulky hydrophobic m-substituent group may be beneficial for the activity (Compound 5 vs. Compound 8). This data indicated the importance of the hydrophobicity and size of the m-substituent on the phenyl ring in LpxH inhibitory activity.

Extended N-acyl groups were well tolerated. Interestingly, the sulfanilide analog (Compound 14) was active, indicating that the indoline ring might not be essential to LpxH inhibition. Surprisingly, the extended sulfanilide analogs (Compound 15 and Compound 16) were less active than Compound 14, and the benzoic acid analogs (compound 19 and Compound 20) were inactive.

The comparison of Compound 1, Compound 17, and Compound 18 revealed that the 6-membered piperazine ring is highly beneficial for the LpxH inhibition by the assessed analog compounds. Surprisingly, the amide analog Compound 43 was inactive, indicating that the sulfonamide linker is important for the Compound 1 activity either by maintaining the optimal compound geometry or by direct interacting with LpxH, or both.

### Example 16: Analysis of SAR of LpxH Targeting Analog Compound

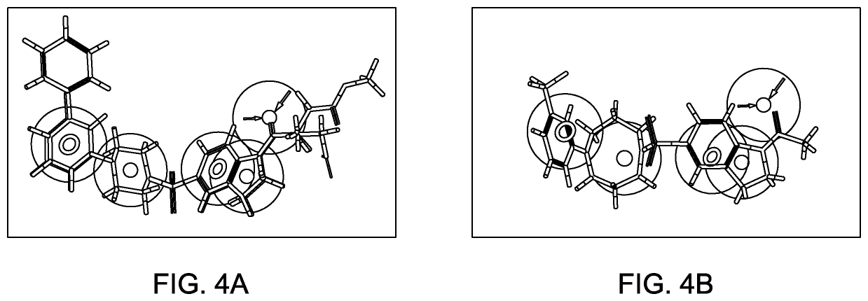

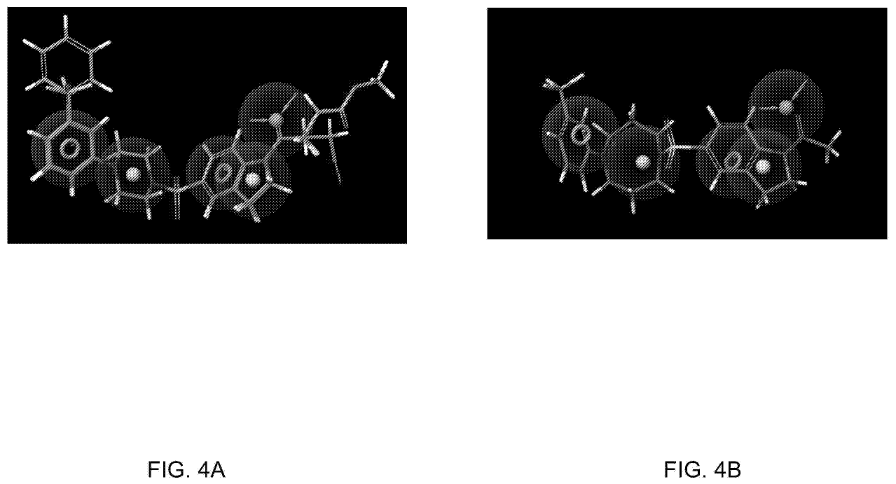

A pharmacophore model of LpxH inhibitors was generated by analyzing the SAR and mapping common structural features of the five most active compounds (>45% inhibition at 1 μM; Compound 1, Compound 5, Compound 6, Compound 7, and Compound 23). A total of 50 five-point hypotheses were generated for LpxH inhibitors, respectively, by requiring all active ligands matched to the generated hypotheses. The initial hypotheses were evaluated by scoring both active and inactive ligands. Although inactive ligands were not involved in model generation, they were used to eliminate hypotheses that do not distinguish between active and inactive compounds, which is especially useful when all active ligands share a common structural scaffold. FIG. 4A shows the pharmacophore with the highest adjusted scores mapped onto the active compound, Compound 1. The pharmacophore model is represented with AHHRR, indicating that it has one hydrogen-bond acceptor (A), two hydrophobic groups (H), and two aromatic rings (R). Two hydrophobic groups and one hydrogen-bonding acceptor are mapped to the piperazine ring, the indoline ring, and the carbonyl group, respectively. These molecular features are shared by all active ligands, whereas inactive analogs, including Compound 18 that has a single atom difference from Compound 1, have poor overlaps with the pharmacophore model of LpxH inhibitors (see FIG. 4B).

The results of the LpxH targeting analog compound disclosed herein were surprising and unexpected in view of the common knowledge in the field of art. Specifically, it was commonly believed based on modeling that the trifluoromethyl substituted phenyl ring is located close to the active site consisting primarily of hydrophilic residues involved in the recognition of the 1-phospho-glucosamine head group and the β-hydroxyl groups of the 2,3-diacyl chains of the product lipid X. Additionally, it was believed that the trifluoromethyl group points toward the solvent-accessible open space above the active site, suggesting that substitution of the trifluoromethyl group with polar functional groups should be well tolerated.

However, the SAR analysis and pharmacophore model of the LpxH targeting analog compound disclosed herein show that replacement of the hydrophobic trifluoromethyl group with a polar functional group (e.g., the hydroxyl group in Compound 9 or the carboxylate group in Compound 11) is detrimental to the inhibitory activity of Compound 1, whereas a bulky hydrophobic substitution (e.g., the bromo group in Compound 5 or the phenyl group in Compound 23) is well tolerated, which is highly unexpected. Furthermore, terminal polar functional groups (such as carboxylates) of the N-acyl chain on the indoline ring (e.g., Compound 6 and Compound 7) were well accepted, and the carbonyl oxygen of the N-acetyl group on the indoline ring was a highlighted hydrogen bond acceptor in pharmacophore model discussed herein, which would likely interact with polar residues in the active site rather than hydrophobic residues in the acyl chain chamber of LpxH. Taken together, the SAR analysis and pharmacophore model discussed herein reveal several inconsistencies of the Compound 1 docking model commonly relied upon by people of ordinary skill in the art. Instead, the Examples disclosed herein supports a model that Compound 1 is oriented with the trifluoromethyl group away from the active site and with the N-acetyl indoline close to the active site.

### Example 17: Compound 1 is a Potent Inhibitor of the LpxH Enzyme from K. pneumonia

The antibiotic activity of Compound 1 against a set of Gram-negative bacteria was evaluated using the bacterial disk diffusion assay. Compound 1 was previously reported to be inactive against wild-type E. coli, Haemophilus influenzae, and Pseudomonas aeruginosa, but displayed notable antibiotic activity against an E. coli mutant strain deficient in the efflux pump (ΔtolC). Similarly, Compound 1 showed no measurable activity against wild-type E. coli (W3110) in disk diffusion assays, but displayed clear killing zones for the E. coli mutant with a compromised efflux pump (W3110 ΔtolC). For example, as seen in FIG. 1B, Compound 1 is inactive against wild-type E. coli, but displays noticeable antibiotic activity against E. coli mutants with efflux pump deletion (ΔtolC) or with a compromised outer membrane (yhjD*ΔkdtA), and against wild-type K. pneumoniae.

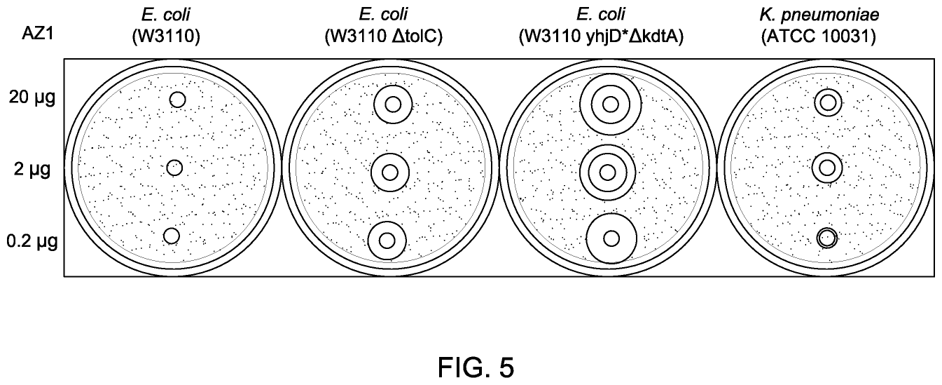

In order to evaluate whether the blockage of compound entry into the bacteria or the efflux of the compound plays a more prominent role in restricting the antibiotic activity of Compound 1, an E. coli strain was constructed containing a leaky outer membrane (W3110 yhjD*ΔkdtA). Similar to the KPM121 strain reported previously, the outer leaflet of the outer membrane of this strain only contains free lipid A molecules lacking all core sugars and O-antigen repeats. Compound 1 had a much larger killing zone against this leaky E. coli strain than the efflux pump deletion strain (W3110 ΔtolC), indicating that outer membrane impermeability plays a more prominent role in limiting the access of Compound 1 to its target than the efflux machinery. Advantageously, Compound 1 exhibited detectable activity against wild-type K. pneumoniae in bacterial disk diffusion assays (see FIG. 5), suggesting that Compound 1 may be an effective inhibitor of K. pneumoniae LpxH in addition to E. coli LpxH.

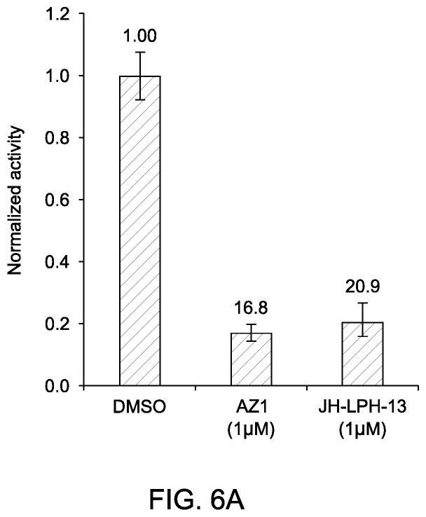

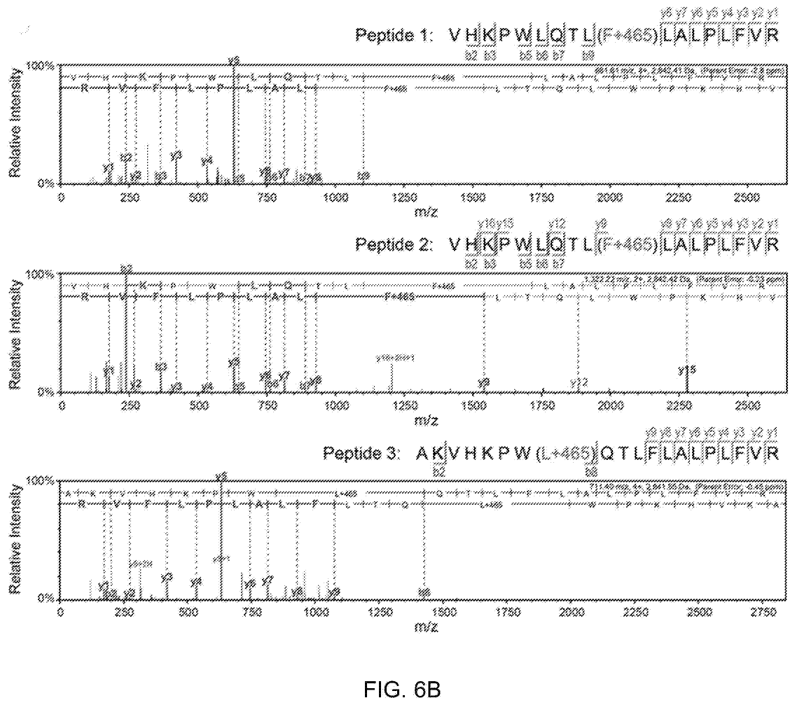

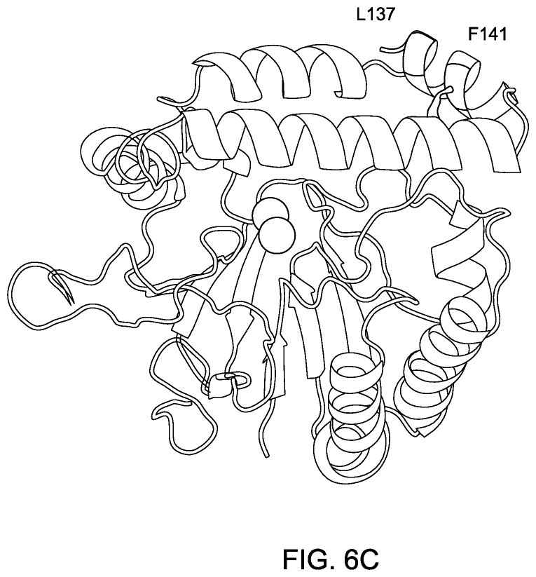

Compound 1 inhibited 75% of the activity of K. pneumoniae LpxH at a concentration of 1 μM (FIG. 1C), similar to that reported for E. coli LpxH previously (83% inhibition of activity at 1 μM Compound 1). Importantly, although purified E. coli LpxH gradually precipitates in solution, K. pneumoniae LpxH was stable and readily yielded protein crystals amenable for structural analysis. Alidation of the binding orientation of Compound 1 bound to E. coli LpxH is shown in FIGS. 6A-6D. FIG. 6A displays similar E. coli LpxH inhibition potency to Compound 1. The error bars represent the standard error of the mean (n=3). FIG. 6B is graphs of the LC-MS/MS analysis of the E. coli LpxH/Compound 12 photo-crosslinking product, which reveals F141 and L137 as the predominant sites of compound crosslinking. FIG. 6C is an image mapping of F141 and L137 onto the modeled E. coli LpxH structure, which confirms that the phenyl group of Compound 1 is located away from the active site. The E. coli LpxH structure was modeled based on the coordinate of H. influenzae LpxH (PDB 5K8K) using SWISS-MODEL (https://swissmodel.expasy.org/). In FIG. 6C, the protein is shown in the cartoon model.

### Example 18: Structure of K. pneumoniae LpxH in Complex with Compound 1

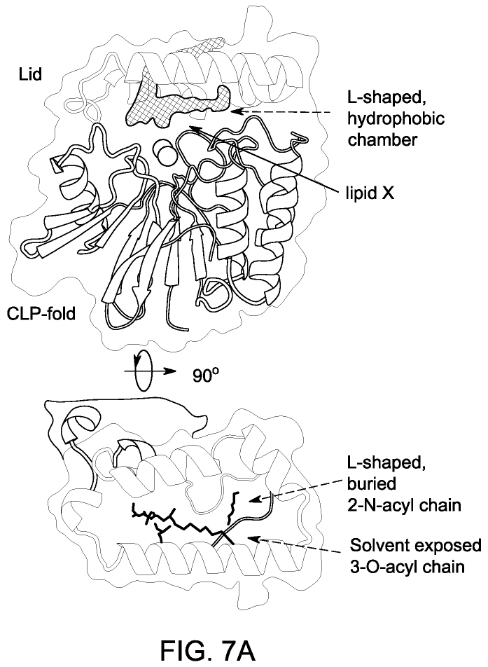

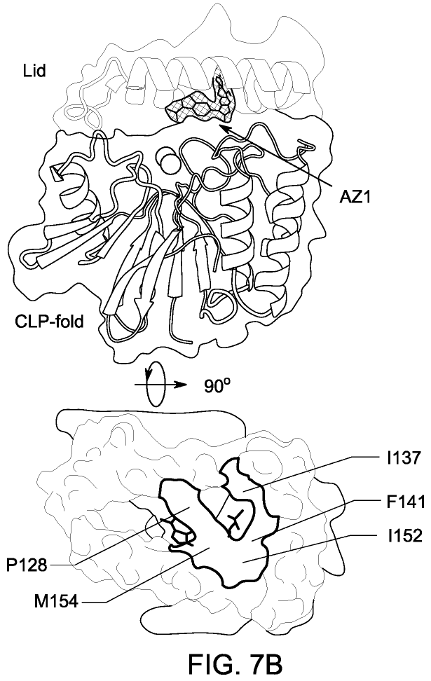

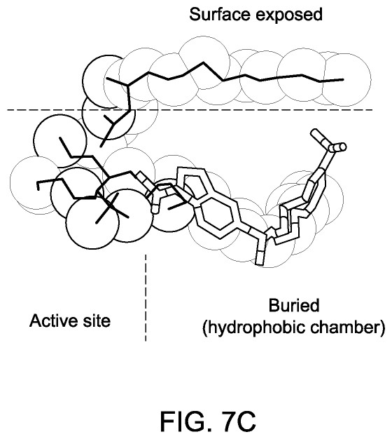

The atomic details of the interaction between K. pneumoniae LpxH and Compound 1 was evaluated. First, K. pneumoniae LpxH was crystallized in complex with its product lipid X and then its structure was determined at 1.92 Å resolution (see FIG. 7A). The overall structure of the K. pneumoniae LpxH-lipid X complex is similar to those of the H. influenzae and P. aeruginosa LpxH-lipid X complexes: it features a central CLP architecture and a unique insertion cap, with a di-manganese cluster coordinated by conserved metal binding residues from the CLP signature motifs in the active site and a prominent L-shaped hydrophobic chamber located adjacent to the active site and between the core CLP domain and the insertion “lid”. The 1-phospho-glucosamine head group of lipid X is recognized in the active site through hydrophilic interactions, whereas the 2-N-acyl chain is buried in the L-shaped hydrophobic acyl-chain binding chamber with the 3-O-acyl chain extending out to the solvent-exposed surface (see FIG. 7A).

By including Compound 1 in early steps of the K. pneumoniae LpxH purification procedure, K. pneumoniae LpxH-Compound 1 complex crystals were obtained that diffracted to 2.26 Å (see FIG. 7B). The density of the Compound 1 is very well defined by the omit density map: it binds exclusively in the acyl-chain-binding chamber of LpxH and is nearly completely shielded from the solvent. The conformation of the LpxH-bound Compound 1 almost overlaps entirely with the L-shaped 2-N-acyl-chain of lipid X, with a sharp kink at the sulfonyl group; the N-acetyl indoline ring of Compound 1 extends towards the active site, and its N-phenyl substituted piperazine group reaches out to the far side of the L-shaped hydrophobic chamber away from the active site (see FIG. 7C). Numerous conserved van der Waals (vdW) interactions are observed between the trifluoromethyl-substituted phenyl group, the piperazine group and the indoline ring of Compound 1 and the surrounding hydrophobic residues (F82, L83, Y125, F128, I137, F141, I152, A153, M156) from K. pneumoniae LpxH (FIG. 2D).

Additionally, the guanidinium sidechain of R80 glides over the indoline ring of Compound 1, forming a classic parallel cation-π stacking interaction. Finally, hydrogen bonds are observed between the Compound 1 acetyl group and the sidechain of N79 of LpxH, and between the Compound 1 sulfonamide group and the sidechain of R157 and the backbone amide group of W46 of LpxH. These structural observations provide an excellent molecular interpretation of our previously reported pharmacophore model of AHHRR, of which HHRR refers to aromatic (H) and hydrophobic (R) groups from the phenyl, piperazine, and indoline moieties of Compound 1, and A refers to the hydrogen bond acceptor of the acetyl carbonyl group. Replacement of the sulfonamide group with an amide group diminishes Compound 1 activity, highlighting the importance of this group for maintaining the proper molecular geometry and hydrogen bonds.

As noted above, the structural analysis of the K. pneumoniae LpxH-Compound 1 complex revealed an Compound 1 binding mode opposite to the docking model commonly used by people of ordinary skill in the art based on the structural analysis of a mutant form of E. coli LpxH, of which the trifluoromethyl phenyl group is located close to the active site. To investigate whether Compound 1 might adopt a different binding orientation when bound to E. coli LpxH, a photo-crosslinking probe of Compound 1 was synthesized by inserting a diazirine group between the trifluoromethyl group and its attached phenyl ring (Compound 12). The diazirine group is a proximity-labeling reagent, which upon UV activation forms a reactive carbene species that crosslinks with nearby residues from the target protein. Compound 12 potently inhibited E. coli LpxH at a concentration of 1 μM (˜80% inhibition; see FIG. 6A), confirming that the introduction of the diazirine group of Compound 12 did not interfere with the compound binding to E. coli LpxH. An LC-MS/MS analysis of the UV-crosslinking product of Compound 12 with E. coli LpxH revealed that F141 was the predominant site of modification (F141^{mod}), with two F141^{mod}-containing peptides accounting for ˜83% of the total ion current of all modified peptide fragments (75.9% of peptide 1 and 7.1% for peptide 2); following F141, residue L137 (1137 in KpLpxH) is also modified (L137^{mod}), with the L137^{mod}-containing peptide accounting for 7.4% of total ion current of modified peptides (see FIGS. 6B and 6C). These three peptide fragments, with modifications at either F141 or L137 in E. coli LpxH, account for >90% of all ion currents for the modified peptides, confirming that the trifluoromethyl phenyl group of Compound 1 is indeed located away from the E. coli LpxH active site (see FIG. 6A)—an observation consistent with our structurally observed binding mode of Compound 1 in the K. pneumoniae LpxH-bound complex.

### Example 19: Optimization from the Cryptic Ligand Envelope

A structural analysis of LpxC inhibitors revealed that there exists substantial ligand dynamics in the inhibitor-bound LpxC complex in solution, even though the corresponding crystal structure reveals a single ligand bound conformation. These multiple ligand conformations in solution together delineate a cryptic ligand envelope that expands the ligand footprint observed in the crystalline state and has enabled the design of novel LpxC inhibitors with significant improvement in the binding affinity and antibiotic activity over the parent compound.

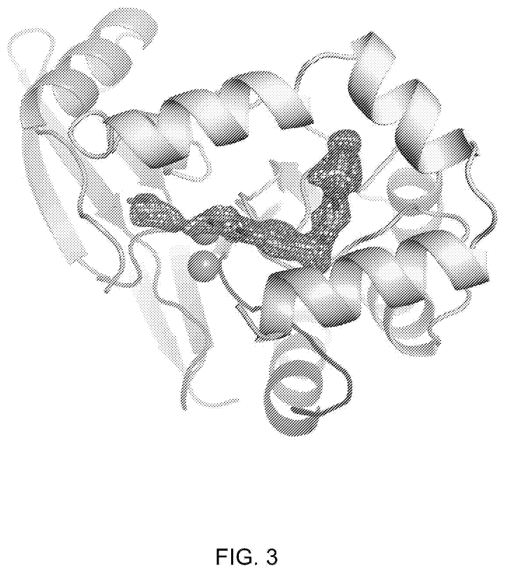

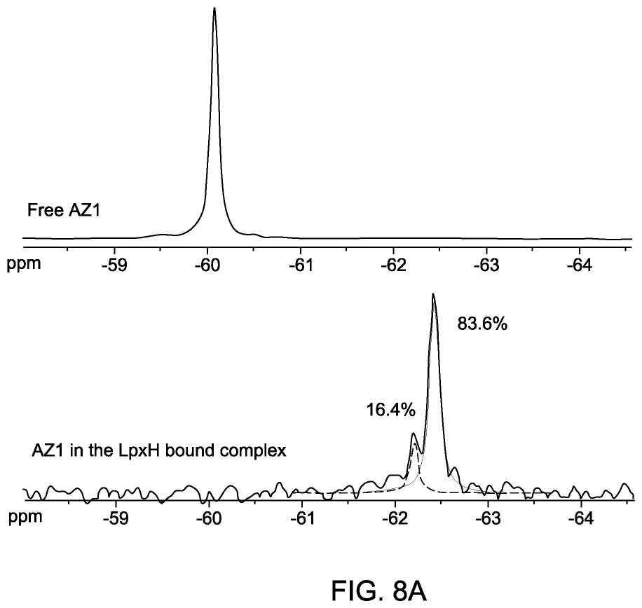

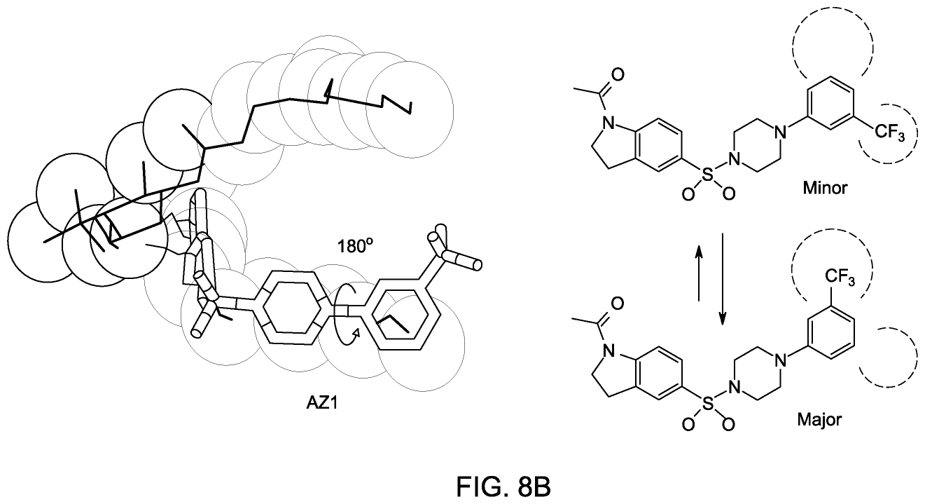

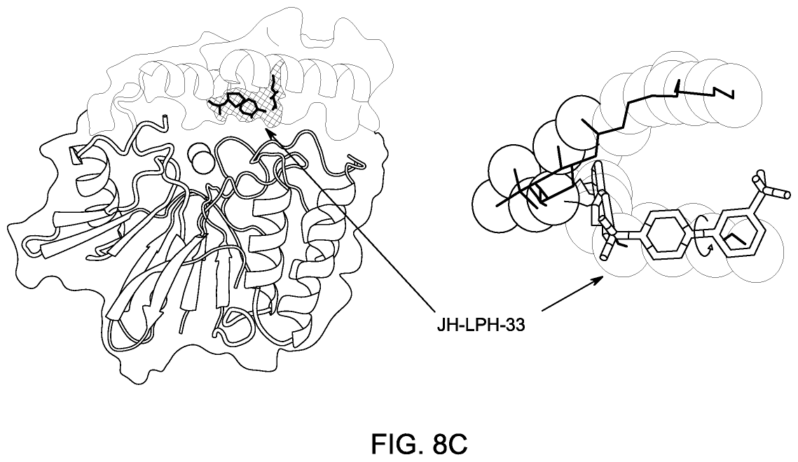

Parallel solution NMR analysis was carried out of Compound 1 bound to K. pneumoniae LpxH. Intriguingly, it was found that despite the observation of a single ligand conformation in the crystal structure of the K. pneumoniae LpxH-Compound 1 complex idetermined at 2.26 Å resolution, the solution ¹⁹F NMR spectrum readily revealed the presence of two ¹⁹F signals of the trifluoromethyl group of Compound 1 when it was bound to K. pneumoniae LpxH (lower panel of FIG. 3A). Fitting of the two signals yielded a major conformation of 83.6% population and a minor conformation of 16.4% population. This is in sharp contrast to the free compound, of which a single ¹⁹F signal was observed (see FIGS. 8A-8C). Taken together, these observations support the notion that Compound 1 binds to K. pneumoniae LpxH in two distinct conformations in solution.

Compound 1 contains a rotatable bond between the trifluoromethyl-substituted phenyl ring and the piperazine group. In order to assess the full interactions the cryptic ligand envelope of Compound 1, two Compound 1 derivatives, namely, Compound 26 and Compound 31, were synthesized that contain either a fluoro- or chloro-substitution at the meta-position of the trifluoromethyl phenyl ring. A crystal structural analysis of Compound 31 in complex with K. pneumoniae LpxH at the 2.25 Å resolution revealed that the binding mode includes the chloro-group filling in the hydrophobic pocket occupied by the terminal methyl group of the 2-N-linked acyl chain shared by the substrate UDP-DAGn and product lipid X, and with the trifluoro-methyl group pointing upward as seen for Compound 1 in the crystal structure (see FIG. 8C).

### Example 20. Sulfonyl Piperazine Analogs Show Enhanced LpxH Inhibition and Antibiotic Activity

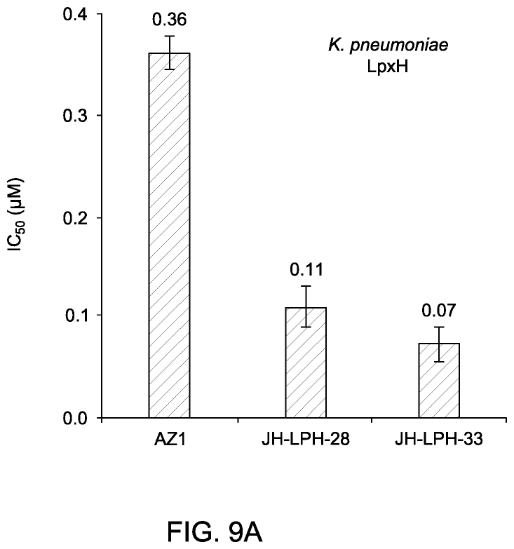

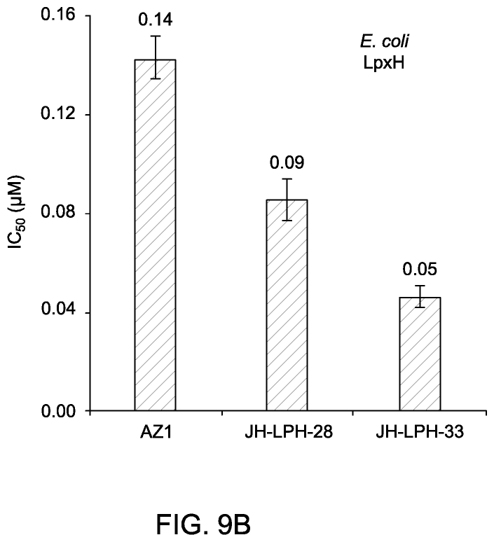

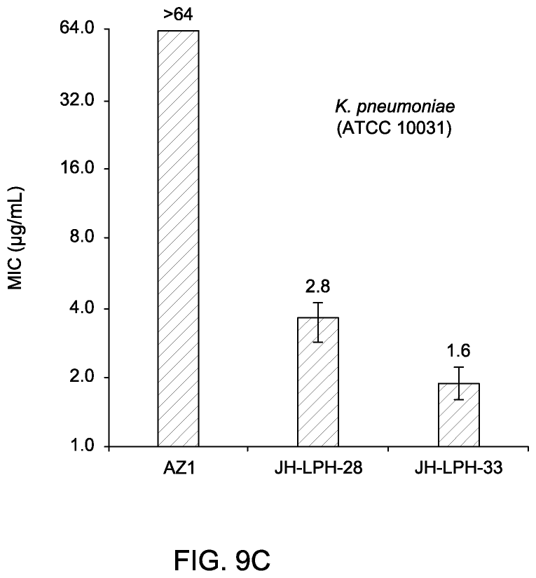

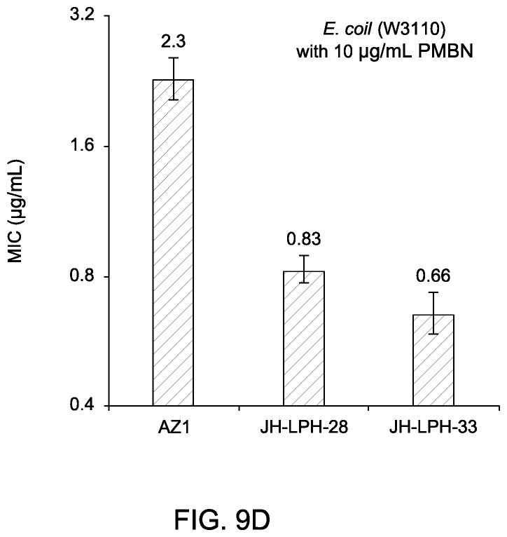

Both of Compound 26 and Compound 31 inhibited LpxH more potently than Compound 1: the fluoro-substituted Compound 26 displayed IC₅₀ values of 0.11 μM against K. pneumoniae LpxH and 0.09 μM against E. coli LpxH; these values were ˜1.5- to 3.3-fold lower than the corresponding IC₅₀ values of Compound 1 (0.36 μM against K. pneumoniae LpxH and 0.14 μM against E. coli LpxH; FIGS. 9A and 9B). The chloro-substituted Compound 31 was even more active, displaying IC₅₀ values of 0.07 μM against K. pneumoniae LpxH and 0.05 μM against E. coli LpxH and improving the potency of Compound 1 by ˜2.8- to 5.1-fold (FIGS. 9A and 9B).

Accompanying the significant enhancement of in vitro inhibition of LpxH, Compound 26 and Compound 31 showed striking improvement in their antibiotic activities. When tested against wild-type K. pneumoniae (ATCC 10031) in cell culture, Compound 1 did not suppress the bacterial growth at 64 μg/mL; in contrast, the fluoro analog Compound 26 potently inhibited the bacterial growth at 2.8 μg/mL and the chloro analog Compound 31 was even more effective, displaying an MIC of 1.6 μg/mL (see FIG. 9C).

Also surprisingly, despite the significant antibiotic activity of Compound 26 and Compound 31 against wild-type K. pneumoniae and their superior inhibition of E. coli LpxH over K. pneumoniae LpxH at nanomolar concentrations in vitro, none of these compounds (including Compound 1) displayed measurable antibiotic activity against wild-type E. coli (data not shown), suggesting that E. coli might be more effective at either blocking the cellular entry of these compounds or at secreting the compounds out through efflux pumps.

It has been previously reported that Compound 1 is inactive for wild-type E. coli, but displays notable antibiotic activity against efflux deficient (ΔtolC) E. coli strains. Interestingly, although the disk diffusion assays confirmed the role of efflux pumps as seen FIG. 5, the disk diffusion assays also revealed that increasing the membrane permeability by compromising the outer membrane of E. coli (i.e. yhjD*ΔkdtA) has a much more pronounced effect on the antibiotic activity of Compound 1 than deleting the efflux pump (i.e. ΔtolC) (FIG. 5).

PMßN itself did not display significant antibiotic activity at concentrations up to 125 μg/mL (data not shown); however, co-administration of PMßN at 10 μg/mL profoundly sensitized the wild-type E. coli strain (W3110) to sulfonyl piperazine LpxH inhibitors. Compound 1, which did not display any detectable effect toward wild-type E. coli by itself, showed robust antibiotic activity with an MIC value of 2.3 μg/mL toward E. coli (see FIG. 9D) when co-administed with PMßN. Likewise, both Compound 26 and Compound 31 displayed outstanding antibiotic activity, with MIC values of 0.83 μg/mL and 0.66 μg/mL respectively (see FIG. 9D). Taken together, these observations provide strong evidence that co-administration of LpxH inhibitors with effective membrane permeability enhancers is an efficient solution for clinical applications of LpxH-targeting antibiotics against multidrug-resistant Gram-negative human pathogens.

Although the crystal structure of the K. pneumoniae LpxH/Compound 1 complex revealed a single compound conformation, the solution NMR study showeds the existence of two distinct compound conformations that together delineate an expanded, cryptic inhibitor envelope invisible in the crystalline state.

### Example 21: Synthesis of the Photo-Crosslinking Probe (Compound 12)

To establish the binding mode of Compound 47, a trifluoromethylaryl diazirine probe (Compound 12, which is shown below) (See Reaction Scheme 8A, below) was synthesized.

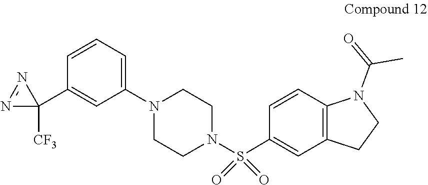 [SMILES: CC(=O)N1CCc2cc(S(=O)(=O)N3CCN(c4cccc(C5(C(F)(F)F)N=N5)c4)CC3)ccc21]

Briefly, trifluoroacetylation of the known aryl bromide provided the trifluoromethyl ketone Compound 48. Compound 48 was converted to the corresponding oxime Compound 49, which was activated by TsCl to afford the tosylate Compound 50. Compound 50 was treated with liquid ammonia to give the diaziridine Compound 51. Oxidation of Compound 51 with freshly prepared Ag₂O provided the diazirine Compound 52. Boc-deprotection of Compound 52 and subsequent coupling of the resulting piperazine Compound 53 with 1-acetyl-5-indolinesulfonyl chloride Compound 54 successfully afforded Compound 12. The compounds referred to in this Example may be seen in Reaction Schemes 8A and 8B.

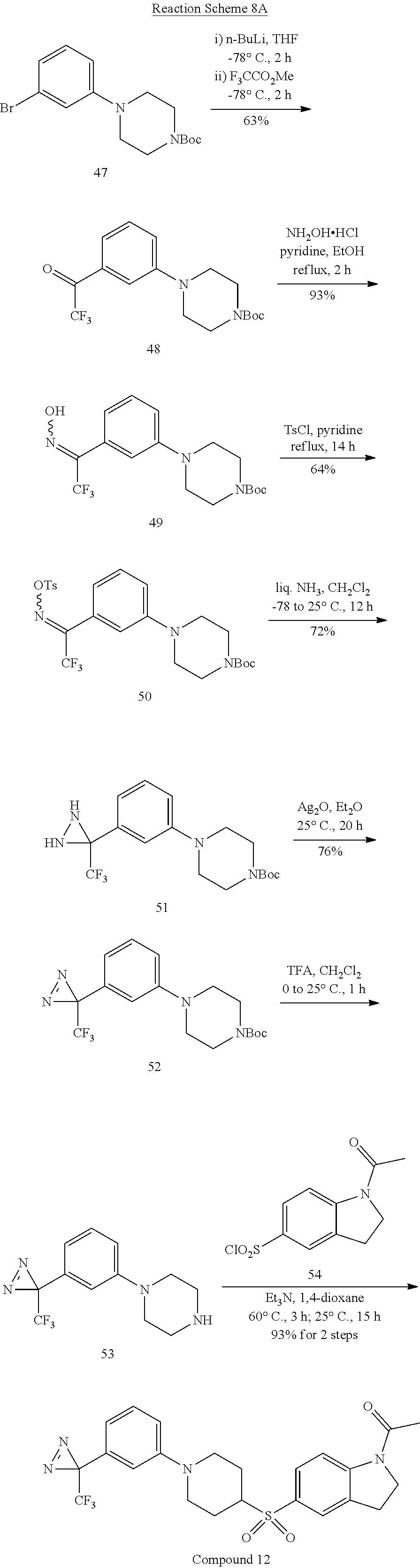

Compound 48: To a solution (−78° C.) of Compound 47 (415 mg, 1.22 mmol) in THF (7 mL) was added dropwise n-BuLi (2.5 M in hexane, 0.73 mL, 1.83 mmol). The reaction mixture was stirred at the same temperature for 2 h before a solution of methyl fluoroacetate (0.16 mL, 1.59 mmol) in THF (3 mL) was added dropwise. After 2 h, the reaction was quenched by addition of saturated aqueous NH₄Cl, and the resulting mixture was extracted with EtOAc. The combined organic layers were washed with brine, dried over anhydrous Na₂SO₄, and concentrated in vacuo. The residue was purified by column chromatography (SiO₂, hexanes/EtOAc, 2/1) to afford Compound 48 (65 mg, 63%). ¹H NMR (400 MHz, CDCl₃) δ 7.54 (m, 2H), 7.42 (t, J=8.0 Hz, 1H), 7.23 (d, J=2.4 Hz, 1H), 3.60 (t, J=5.2 Hz, 4H), 3.20 (t, J=5.2 Hz, 4H), 1.48 (s, 9H); HRMS (ESI) m/z 359.1571 [(M+H)⁺, C₁₇H₂₁F₃N₂O₃ requires 359.1577].

Compound 49: To a solution of Compound 48 (71 mg, 0.2 mmol) in pyridine/EtOH (2/1, 1.5 mL) was added hydroxylamine hydrochloride (18 mg, 0.26 mmol). The reaction mixture was heated at 120° C. for 2 h and concentrated in vacuo. The resulting residue was partitioned between water and EtOAc. The aqueous layer was extracted with EtOAc, and the combined organic layers were dried over anhydrous Na₂SO₄ and concentrated in vacuo. The residue was purified by column chromatography (SiO₂, hexanes/EtOAc, 2/1) to afford Compound 49 (70 mg, 93%). ¹H NMR (400 MHz, CDCl₃) δ 10.07 (s, 1H), 7.38-7.32 (m, 1H), 7.02-6.97 (m, 3H), 3.60 (t, J=4.8 Hz, 4H), 3.13 (t, J=4.8 Hz, 4H), 1.49 (s, 9H); HRMS (ESI) m/z 374.1688 [(M+H)⁺, C₁₇H₂₂F₃N₃O₃ requires 374.1686].

Compound 50: To a solution of Compound 49 (101 mg, 0.27 mmol) in pyridine (3 mL) was added TsCl (78 mg, 0.41 mmol). The reaction mixture was refluxed for 14 h and concentrated in vacuo. The resulting residue was partitioned between water and EtOAc. The aqueous layer was extracted with EtOAc. The combined organic layers were washed with brine, dried over anhydrous Na₂SO₄, and concentrated in vacuo. The residue was purified by column chromatography (SiO₂, hexanes/EtOAc, 2/1) to afford Compound 50 (91 mg, 64%). ¹H NMR (400 MHz, CDCl₃) δ 7.87 (d, J=8.4 Hz, 2H), 7.38 (d, J=8.4 Hz, 2H), 7.34 (m, 1H), 7.04 (d, J=8.0 Hz, 1H), 6.83 (m, 2H), 3.59 (t, J=4.8 Hz, 4H), 3.14 (t, J=4.8 Hz, 4H), 2.48 (s, 3H), 1.49 (s, 9H); HRMS (ESI) m/z 550.1588 [(M+Na)⁺, C₂₄H₂₈F₃N₃O₅S requires 550.1594].

Compound 51: To a cooled (−78° C.) solution of Compound 50 (84 mg, 0.16 mmol) in CH₂Cl₂ (1.6 mL, 0.16 M) was bubbled NH₃ gas. The resulting mixture was stirred in a sealed tube for 5 min at −78° C. and slowly warmed to 25° C. After stirring for 12 h at the same temperature, the reaction mixture was cooled to −78° C., and ammonia was evaporated for 5 h at 25° C. The solid was filtered off, and the filtrate was washed with water and extracted with CH₂Cl₂. The combined organic layers were washed with brine, dried over anhydrous Na₂SO₄, and concentrated in vacuo. The residue was purified by column chromatography (SiO₂, hexanes/EtOAc, 2/1) to afford Compound 51 (43 mg, 72%). ¹H NMR (400 MHz, CDCl₃) δ 7.30 (t, J=8.0 Hz, 1H), 7.14 (s, 1H), 7.10 (d, J=8.0 Hz, 1H), 6.98 (d, J=8.0 Hz, 1H), 3.58 (t, J=4.4 Hz, 4H), 3.16 (t, J=4.4 Hz, 4H), 2.76 (d, J=8.0 Hz, 1H), 2.23 (d, J=8.0 Hz, 1H), 1.48 (s, 9H); HRMS (ESI) m/z 373.1850 [(M+H)⁺, C₁₇H₂₃F₃N₄O₂ requires 373.1846].

Compound 52: To a solution of Compound 51 (42 mg, 0.11 mmol) in dry Et₂O (1.1 mL, 0.1 M) was added a freshly prepared Ag₂O (76 mg, 0.33 mmol) at 25° C. After stirring for 20 h at the same temperature, the reaction mixture was filtered. The organic layers were dried over anhydrous Na₂SO₄ and concentrated in vacuo. The residue was purified by column chromatography (SiO₂, hexanes/EtOAc, 5/1) to afford Compound 52 (31 mg, 76%). ¹H NMR (400 MHz, CDCl₃) δ 7.23 (d, J=7.6 Hz, 1H), 6.91 (d, J=8.4 Hz, 1H), 6.68 (d, J=7.6 Hz, 1H), 6.58 (s, 1H), 3.54 (t, J=4.8 Hz, 4H), 3.09 (t, J=4.8 Hz, 4H), 1.44 (s, 9H); HRMS (ESI) m/z 371.1692 [(M+H)⁺, C₁₇H₂₁F₃N₄O₂ requires 371.1689].

Compound 12: To a cooled (0° C.) solution of Compound 52 (30 mg, 0.081 mmol) in dry CH₂Cl₂ (1 mL, 0.08 M) was added dropwise TFA (0.5 mL). After stirring for 40 min at 0° C., the solvents were removed under reduced pressure to give Compound 53, which was used in the next step without further purification. Compound 7 was dissolved in 1,4-dioxane (1 mL, 0.08 M), and the mixture was treated with Et₃N (0.014 mL, 0.097 mmol) and heated until the temperature reached 60° C. 1-Acetyl-5-indolinesulfonyl chloride (Compound 54, 21 mg, 0.081 mmol) in 1,4-dioxane (0.5 mL) was added dropwise to the reaction mixture, which was stirred at 60° C. for 3 h and then at 25° C. overnight. Water was added, and the aqueous layer was extracted with EtOAc. The combined organic layers were dried over anhydrous Na₂SO₄ and concentrated in vacuo. The residue was purified by column chromatography (SiO₂, CH₂Cl₂/MeOH, 10/1) to afford Compound 12 as a yellow powder (37 mg, 93%). ¹H NMR (400 MHz, CDCl₃) δ 8.43 (d, J=8.4 Hz, 1H), 7.62 (d, J=8.4 Hz, 1H), 7.56 (s, 1H), 7.26 (t, J=8.0 Hz, 1H), 6.88 (d, J=8.0 Hz, 1H), 6.72 (d, J=8.0 Hz, 1H), 6.56 (s, 1H), 4.16 (t, J=8.8 Hz, 2H), 3.30-3.32 (m, 6H), 3.14 (m, 4H), 2.27 (s, 3H); HRMS (ESI) m/z 494.1469 [(M+H)⁺, C₂₂H₂₂F₃N₅O₃S requires 494.1468].

### Example 22: Synthesis of Compound 1 and Analogs Compound 26 and Compound 31

Compound 47 was synthesized as described previously. The analogs of Compound 47, Compound 26 and Compound 31, were prepared by coupling known substituted phenyl piperazines with 1-acetyl-5-indolinesulfonyl chloride (see Reaction Scheme 8B). The compounds referred to in this Example may be seen in Reaction Schemes 8A and 8B.

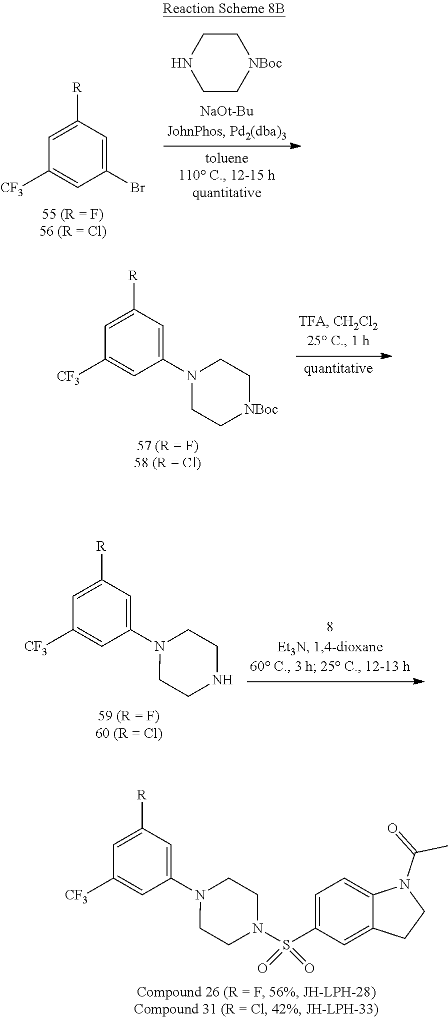

Compound 57: Toluene (0.75 mL) was added to 1-bromo-3-fluoro-5-(trifluoromethyl)benzene (Compound 55, 76 mg, 0.31 mmol), 1-(tert-butyloxycarbonyl)piperazine (75 mg, 0.4 mmol), NaOt-Bu (36 mg, 0.37 mmol), JohnPhos (7.5 mg, 8 mol %), and Pd₂(dba)₃ (11.5 mg, 4 mol %). Argon was bubbled through the reaction mixture for 15 min, and the reaction mixture was refluxed for 12 h. The reaction mixture was cooled and concentrated in vacuo. The residue was dissolved in CH₂Cl₂/MeOH (1/1), filtered through Celite, and concentrated in vacuo. The residue was purified by column chromatography (SiO₂, hexanes/EtOAc, 20/1) to quantitatively afford Compound 57 as a yellow solid. ¹H NMR (400 MHz, CDCl₃) δ 6.89 (s, 1H), 6.78 (s, 1H), 6.72 (d, J=10.4 Hz, 1H), 3.59 (br s, 4H), 3.20 (br s, 4H), 1.49 (s, 9H).

Compound 59: To a solution of Compound 57 (45 mg, 0.13 mmol) in CH₂Cl₂ (0.65 mL) was added TFA (0.26 mL). After stirring for 1 h at 25° C., the reaction mixture was concentrated to quantitatively provide Compound 59 as a yellow solid. ¹H NMR (400 MHz, CD₃OD) δ 7.11 (s, 1H), 7.04 (dd, J=11.7, 2.2 Hz, 1H), 6.91 (d, J=8.2 Hz, 1H), 3.54-3.51 (m, 4H), 3.39-3.37 (m, 4H); HRMS (ESI) m/z 249.1009 [(M+H)⁺, C₁₁H₁₂F₄N₂ requires 249.1009].

Compound 26: A solution of Compound 59 (17 mg, 0.068 mmol) and Et₃N (0.01 mL, 0.08 mmol) in 1,4-dioxane (1 mL) was heated to 60° C. and 1-acetyl-5-indolinesulfonyl chloride (Compound 54, 17.7 mg, 0.068 mmol) in dioxane (1 mL) was added dropwise. After stirring at 60° C. for 3 h, the reaction mixture was cooled to 25° C. and stirred for 12 h. Water was added, and the reaction mixture was extracted with EtOAc. The combined organic layers were dried over anhydrous Na₂SO₄ and concentrated in vacuo. The residue was purified by column chromatography (SiO₂, hexanes/EtOAc, 1/1) to afford Compound 26 (18 mg, 56%) as a white solid. ¹H NMR (400 MHz, CDCl₃) δ 8.34 (d, J=9.0 Hz, 1H), 7.63 (d, J=8.6 Hz, 1H), 7.57 (s, 1H), 6.82 (s, 1H), 6.78 (d, J=7.9 Hz, 1H), 6.66 (d, J=11.3 Hz, 1H), 4.16 (t, J=8.7 Hz, 2H), 3.32-3.26 (m, 6H), 3.16-3.13 (m, 4H), 2.27 (s, 3H); HRMS (ESI) m/z 472.1318 [(M+H)⁺, C₂₁H₂₁F₄N₃O₃S requires 472.1313].

Compound 57: Toluene (5.8 mL) was added to 1-bromo-3-chloro-5-(trifluoromethyl)benzene (Compound 56, 500 mg, 1.93 mmol), 1-(tert-butyloxycarbonyl)piperazine (466 mg, 2.5 mmol), NaOt-Bu (278 mg, 2.9 mmol), JohnPhos (58 mg, 10 mol %), and Pd₂(dba)₃ (88 mg, 5 mol %). Argon was bubbled through the reaction mixture for 15 minutes, and the reaction mixture was refluxed for 15 h. The reaction mixture was concentrated in vacuo and the residue was dissolved in CH₂Cl₂/MeOH (1/1), filtered through Celite, and concentrated in vacuo. The residue was purified by column chromatography (SiO₂, hexanes/EtOAc, 20/1) to afford Compound 58, quantitatively. ¹H NMR (400 MHz, CDCl₃) δ 7.06 (s, 1H), 7.00 (s, 1H), 6.97 (s, 1H), 3.60-3.57 (m, 4H), 3.21-3.19 (m, 4H), 1.48 (s, 9H).

Compound 60: TFA (0.1 mL) was added to a solution of Compound 58 (19.5 mg, 0.05 mmol) in CH₂Cl₂ (0.27 mL) and stirred for 1 h. The reaction mixture was concentrated in vacuo to quantitatively afford Compound 60. ¹H NMR (400 MHz, CD₃OD) δ 7.29 (s, 1H), 7.21 (s, 1H), 7.16 (s, 1H), 3.54-3.51 (m, 4H), 3.39-3.36 (m, 4H); HRMS (ESI) m/z 265.0718 [(M+H)⁺, C₁₁H₁₂ClF₃N₂ requires 265.0714].

Compound 31: A solution of Compound 60 (14 mg, 0.053 mmol) and Et₃N (0.01 mL, 0.063 mmol) in 1,4-dioxane (0.78 mL) was heated to 60° C. and 1-acetyl-5-indolinesulfonyl chloride (Compound 54, 13.8 mg, 0.053 mmol) in dioxane (1 mL) was added dropwise. After stirring at 60° C. for 3 h, the reaction mixture was cooled to 25° C. and stirred for 13 h. Water was added, and the reaction was extracted with EtOAc. The combined organic layer was dried over anhydrous Na₂SO₄ and concentrated in vacuo. The residue was purified by column chromatography (SiO₂, hexanes/EtOAc, 1/1) to afford Compound 31 (11 mg, 42%) as a white solid. ¹H NMR (400 MHz, CDCl₃) δ 8.33 (d, J=8.5 Hz, 1H), 7.62 (d, J=8.5 Hz, 1H), 7.56 (s, 1H), 7.05 (s, 1H), 6.94 (s, 1H), 6.91 (s, 1H), 4.15 (t, J=8.6 Hz, 2H), 3.31-3.25 (m, 6H), 3.15-3.14 (m, 4H), 2.26 (s, 3H); HRMS (ESI) m/z 488.1018 [(M+H)⁺, C₂₁H₂₁ClF₃N₃O₃S requires 488.1017].

### Example 23: Construction of the E. coli Mutant Strain with a Leaky Outer Membrane

To construct the E. coli mutant strain with a compromised outer membrane, the R₁₃₄C point mutation in the yhjD gene that suppresses the lethal phenotype of a kdo-deficient strain was utilized. Because yhjD is not an essential gene, it was first replaced with a Cam-SacB cassette derived from plasmid pYA4373 in the wild-type E. coli strain (W3110) using the one-step chromosomal gene inactivation protocol with the helper plasmid pKD46. The Cam-SacB cassette was then replaced with a yhjD-R134C mutated DNA fragment with the same strategy using sucrose as the counter selection to yield strain W3110 yhjD*. The chromosomal deletion of the essential gene kdtA was first constructed in strain DY330 carrying a plasmid overexpressing MsbA except that the kdtA gene was replaced with a removable kanamycin cassette used in constructing the E. coli Keio collection, a single-gene knockout mutant library. After transferring ΔkdtA::kan into W3110 yhjD* by P1 transduction, the kanamycin cassette was removed using the helper plasmid pCP20 and the mutated strain was designated W3110 yhjD*ΔkdtA. The mutation in yhjD and deletion of kdtA were verified by PCR and sequencing.

### Example 24: Bacterial Disk Diffusion Assays

Bacterial strains of wild-type K. pneumoniae (ATCC 10031), wild-type E. coli (W3110), E. coli with the efflux pump deletion (W3110 ΔtolC), and E. coli with a leaky outer membrane (W3110 yhjD*ΔkdtA) were used. Each strain was grown overnight in the Lysogeny Broth (LB) media, and 1 μL of the overnight culture was diluted to 100 μL. The diluted culture was spread on a LB plate with sterile cotton-tipped wooden applicators. Small filter paper disks were placed on the plate and different amounts of Compound 1 (20, 2 and 0.2 μg) were spotted onto the disks. After incubation overnight at 37° C., the plates were imaged for the zone of inhibition.

### Example 25: Measurements of the Minimum Inhibitory Concentration (MIC)

The MIC assay protocol was adapted from methods described in National Committee for Clinical Laboratory Standards (NCCLS) to using 96-well plates. Bacteria strains of wild-type K. pneumoniae (ATCC 10031) and wild-type E. coli (W3110) were grown in the Cation Adjusted Mueller-Hinton medium at 37° C. in the presence of varying concentrations of inhibitors and 7% DMSO. MICs were reported as the lowest compound concentration that inhibited bacterial growth.

### Example 26: Construction and Purification of GB1-E. coli LpxH-His₁₀ and GB1-K. pneumoniae LpxH-His₁₀ for Enzymatic Assays

GB1-fused LpxH constructs with enhanced stability were consturcted for enzymatic assays. Briefly, genes encoding E. coli and K. pneumoniae LpxH enzymes were cloned into a modified pET30 vector (EMD Millipore) containing GB1 between the NdeI and BamHI sites and a His₁₀ tag after the BamHI site. The DNA fragments encoding different LpxH orthologs were inserted immediately after GB1 with the infusion cloning method, so the final expression construct contained GB1-E. coli LpxH-His₁₀ or GB1-K. pneumoniae LpxH-His₁₀. The constructs were verified by DNA sequencing and were used to transform BL21 STAR (DE3) competent E. coli cells (ThermoFisher) for expression of LpxH.

Cells were grown in the LB media at 37° C. until OD₆₀₀ reached 0.5, induced with 1 mM isopropyl-β-D-thiogalactopyranoside (IPTG) for 3 hours, and then harvested by centrifugation. All of the purification steps were carried out at 4° C. Bacterial cell pellets from 8 L of induced culture were resuspended and lysed in 120 mL of the lysis buffer containing 20 mM HEPES (pH 8.0) and 200 mM NaCl using French Press. Cell debris were removed by centrifugation at 10,000×g for 40 minutes. To the supernatant, n-Dodecyl-β-D-Maltopyranoside (DDM) was added to reach a final concentration of 1.5% (w/v; 29 mM). After 2 hours of incubation, the insoluble fraction was removed by ultra-centrifugation at 100,000×g for 1 hour. Supernatant from the centrifugation was diluted to a final volume of 240 mL with the lysis buffer and added to a column containing 20 mL of HisPur™ Ni-NTA resin (ThermoFisher) pre-equilibrated with 100 mL of the purification buffer containing 20 mM HEPES (pH 8.0), 200 mM NaCl, and 0.0174% (w/v; 0.34 mM) DDM. The column was washed with 250 mL of the purification buffer containing 50 mM imidazole, and the LpxH was eluted with 150 mL of the purification buffer containing 300 mM imidazole. The eluted protein sample was concentrated and purified to homogeneity with size-exclusion chromatography (Superdex 200; GE Healthcare Life Sciences) in the purification buffer. Peak fractions were concentrated to ˜2 mg/mL. The stock enzyme solution was mixed with glycerol (50% v/v final concentration), aliquoted, and flash-frozen with liquid nitrogen for storage at −80° C.

### Example 27: LpxE-Coupled LpxH Activity Assay

The LpxE-coupled LpxH activity assay was conducted as described above. Briefly, two reaction mixtures were prepared that contain 20 mM Tris-HCl (pH 8.0), 0.5 mg/mL BSA, 0.02% Triton X-100, 1 mM MnCl₂, and 10% DMSO, either with 100 μM substrate (UDP-DAGn) or with LpxH (10 ng/mL of E. coli LpxH or 20 ng/mL of K. pneumoniae LpxH) and the desired concentration of the inhibitor. The reaction mixtures were pre-incubated at 37° C. for 10 minutes before an equal volume of the LpxH mixture was added to the substrate mixture to start the reaction at 37° C. At the desired reaction time points, an aliquot of 20 μL reaction mixture was removed and added to a well in 96-well half-area plate containing 5 mM EDTA (final concentration) to quench the LpxH reaction. Purified Aquifex aeolicus LpxE was then added to a final concentration of 5 μg/mL. The plate was incubated at 37° C. for 30 minutes followed by addition of formic acid to a final concentration of 3.75 M to quench the reaction. The malachite green reagent was added with a 5-fold dilution and the absorbance at 620 nm was measured after 30-minute incubation at room temperature. The IC₅₀ value was extracted from fitting of the dose response curve of vᵢ/v₀=1/(1+[I]/IC₅₀).

### Example 28: Construction and Purification of K. pneumoniae LpxH-TEV-His10 for Structural Analysis

For crystallographic studies, K. pneumoniae LpxH was cloned into a modified pET21 b (Novagen/MilliporeSigma) vector, yielding the LpxH fusion protein with a C-terminal TEV protease site (ENLYFQGS) and His₁₀ tag, similar to the previously reported H. influenzae LpxH construct. The K. pneumoniae LpxH construct used for crystallization of lipid X-bound and Compound 1-bound complexes contained a valine residue immediately after the start codon due to a cloning artifact, which was eliminated through mutagenesis in the construct used for crystallization of the Compound 31-bound complex.

Vector-transformed BL21 STAR (DE3) E. coli cells (Thermo Fisher Scientific) were grown in the LB media at 37° C. until OD₆₀₀ reached 0.5, induced with 1 mM isopropyl-β-D-thiogalactopyranoside (IPTG) for additional 5 hours at 30° C., and then harvested by centrifugation. Protein purification was carried out at 4° C. Cell pellets from 8 L of induced culture were resuspended and lysed in 120 mL of the lysis buffer containing 50 mM phosphate-citrate, 20 mM MES (pH 6.0), 600 mM NaCl, 10% sucrose, 5 mM 2-mercaptoethanol, 10 mM imidazole, and 0.1% Triton X-100 using a French Pressure cell. Cell debris were removed by centrifugation at 10,000×g for 40 minutes, and the supernatant was loaded onto a column containing 20 mLs of HisPur™ Ni-NTA resin (ThermoFisher) pre-equilibrated with 100 mL of the lysis buffer. After extensive column wash with the purification buffer containing 20 mM phosphate-citrate, 20 mM MES (pH 6.0), 300 mM NaCl, 5% glycerol, 5 mM 2-mercaptoethanol, and 40 mM imidazole, LpxH was eluted from the column with a stepwise increase of imidazole from 40-400 mM in the purification buffer. The sample was concentrated for further purification with size-exclusion chromatography (Superdex 200; GE Healthcare Life Sciences) in the FPLC buffer containing 20 mM MES (pH 6.0), 800 mM NaCl, 1 mM DTT, and 5% glycerol. The peak fractions containing K. pneumoniae LpxH were buffer exchanged into a buffer containing 20 mM MES (pH 6.0), 200 mM NaCl, 1 mM DTT, and 5% glycerol and concentrated to 8 mg/mL for crystallization.

Protein crystals were grown using the sitting-drop vapor diffusion method at 20° C. Each drop was prepared by mixing 1 μL of the protein solution with 1 μL of the reservoir solution containing 200 mM calcium chloride dihydrate, 100 mM HEPES (pH 7.0), 33% PEG 400. Diffraction quality protein crystals were typically harvested after 2 weeks and soaked with the reservoir solution additionally containing 20% glycerol and 100 μM MnCl₂ for cryoprotection.

In order to obtain K. pneumoniae LpxH co-crystals with sulfonyl piperazine antibiotics, individual compounds were incubated with K. pneumoniae LpxH after the Ni-NTA affinity column purification step for 30 minutes. The complex sample was then purified to homogeneity by size-exclusion chromatography (Superdex 200; GE Healthcare) as described above and concentrated to 4-8 mg/mL for co-crystallization. An excess amount of compound in DMSO (2 molar equivalent to K. pneumoniae LpxH) was added before setting up crystal trays, yielding a protein-compound solution containing 20 mM MES (pH 6.0), 200 mM NaCl, 1 mM DTT, 5% glycerol, and 1.5% DMSO. Diffraction-quality crystals were obtained using the sitting-drop vapor diffusion method at 20° C. by mixing 1 μL of the protein solution with 1 μL of the reservoir solution containing 50 mM sodium chloride, 20 mM magnesium chloride hexahydrate, 100 mM sodium citrate (pH 6.0), and 22% PEG 400. The LpxH-compound co-crystals were typically harvested after 2 weeks and were soaked with the reservoir solution containing 20% glycerol and 100 μM MnCl₂ for cryoprotection.

### Example 29: Analysis of K. pneumoniae LpxH Complexes with Lipid X, Compound 1, and Compound 31

Data sets of the K. pneumoniae LpxH complexes with lipid X, Compound 1, and Compound 31 were collected at the Northeastern Collaborative Access Team (NECAT) 24-ID-C and 24-ID-E beamlines at the Advanced Photon Source at Argonne National Laboratory. The X-ray diffraction data were processed using XDS. The phase information of the crystal structures of the K. pneumoniae LpxH complexes was obtained by molecular replacement with the PHASER module in the PHENIX suite using the PDB entry 5K8K as the search model. Restraints of the inhibitors were generated by using eLBOW and edited manually. Iterative model building and refinement was carried out using COOT and PHENIX. The 2mFo-DFc omit maps were generated using PHENIX.

For photo-crosslinking experiment for identification of binding mode, purified E. coli LpxH at a concentration of 50 μM in 20 mM HEPES (pH 8.0), 200 mM NaCl, 0.2 mM DTM, and 2% DMSO was incubated with 100 μM of Compound 12, the Compound 1 photo-crosslinking probe, for 30 minutes at 4° C. After incubation, the reaction mixture was illuminated at 365 nm at 4° C. for 10 minutes. The crosslinked sample was then trypsinized and the resulting peptide fragments were analyzed by LC-MS/MS to identify residues modified by the photo-crosslinking probe Compound 12.

For Solution NRM measurements, purified K. pneumoniae LpxH at the 20 μM concentration in a buffer containing 800 mM NaCl, 5% glycerol, and 20 mM MES (pH 6.0) was incubated with a 5-fold molar ratio of Compound 1 with a final DMSO concentration of 10% for 2 hours at 4° C. The sample was then concentrated and buffer exchanged 200-fold with the above mentioned buffer containing 5% DMSO. The resulting protein complex sample was concentrated to 150 μM and loaded into a 4 mm NMR tube, which was inserted into a 5 mm NMR tube with D20 added between the two NMR tubes as the lock. For the Compound 1 compound control, 100 μM Compound 1 in DMSO was loaded into a 5 mm NMR tube. ¹⁹F spectra were collected at 25° C. on a 500 MHz Bruker spectrometer equipped with a ¹⁹F cryoprobe. NMR data were processed using NMRPipe, and figures were generated using iNMR (www.inmr.net).

The X-ray structure coordinates of the K. pneumoniae LpxH complexes with lipid X, Compound 1, and Compound 31 have been deposited to the PDB with accession codes of 6PH9, 6PIB, and 6PJ3, respectively.

1. A compound of Formula (I) or a salt thereof: 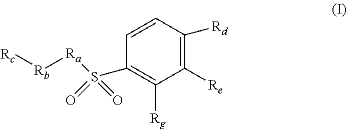
wherein
Rₐ is selected from the group consisting of: 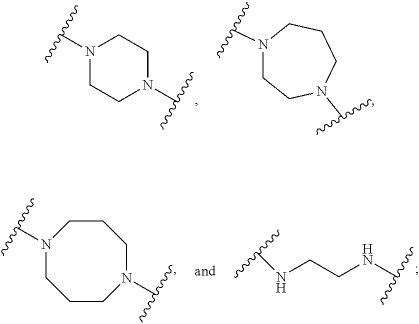 [SMILES: *N1CCN(*)CC1 ; *N1CCCN(*)CC1 ; *N1CCCN(*)CCC1 ; *NCCN*]
R_{b} is selected from the group consisting of single bond, C₄ to C₁₀ unsubstituted or substituted aryl, unsubstituted or substituted four to ten member heterocycle ring, and C₁ to C₁₀ unsubstituted or substituted alkyl;
R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, or C₁ to C₁₀ unsubstituted or substituted alkyl;
R_{d} and Rₑ are each independently hydrogen, —OH, —COH, —COH, —COC, —COOH, R_{f}, or are taken together as an unsubstituted or substituted four to eight member nitrogen containing heterocycle ring,
wherein R_{f} is selected from the group consisting of: 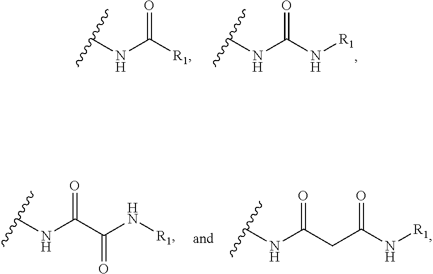
R₁ is hydrogen, methyl, —OH, C₁ to C₁₀ alkyl unsubstituted or substituted with —OH, —COH, —COH, —COC, —NHOH, —CONHOH, —COOH, an unsubstituted or substituted four to eight member heterocycle ring, or a combination thereof, and
R_{g} is hydrogen, —OH, —COH, —COH, —COC, —COOH, or C₁ to C₅ unsubstituted or substituted alkyl.

2. The compound of 1, wherein R_{d} and Rₑ taken together as an unsubstituted or substituted four to eight member nitrogen containing heterocycle ring.

3. The compound of 2, wherein R_{d} and Rₑ taken together as an unsubstituted or substituted five member nitrogen containing heterocycle ring.

4. The compound of 3, wherein the compound of Formula (I) has a structure according to Formula (II): 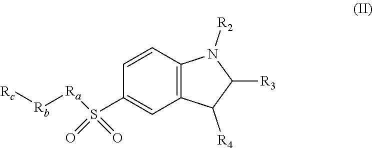
wherein,
R₂ is a hydrogen, an aldehyde, a ketone, or a C₁ to C₅ alkyl unsubstituted or substituted with —OH, —COH, —COC, —COOH, or a combination thereof, and
R₃ and R₄ are independently hydrogen, C₁ to C₅ substituted or unsubstituted alkyl, or taken together as a double bond.

5. The compound of claim 1, wherein R₂ is a hydrogen, —COH, —COC, or —COOH.

6. The compound of claim 1, wherein R₃ and R₄ are hydrogen or taken together as a double bond.

7. The compound of claim 1, wherein R_{d} is R_{f}, Rₑ is hydrogen, —COH, —COH, —COC, or —COOH, and R_{g} is hydrogen.

8. The compound of claim 7, wherein R_{d} is R_{f} and Rₑ is hydrogen.

9. The compound of claim 7, wherein R₁ is a C₁ to C₁₀ alkyl substituted with —OH, —COH, —COH, —COC, —NHOH, —CONHOH, —COOH, or a combination thereof.

10. The compound of claim 9, wherein R₁ is a C₁ to C₈ alkyl substituted with —OH, —COH, —COH, —COC, —NHOH, —CONHOH, or —COOH.

11. The compound of claim 10, wherein R₁ is a C₁ to C₆ alkyl substituted with —OH, —COH, —COH, —COC, —NHOH, —CONHOH, or —COOH.

12. The compound of claim 11, wherein R₁ is a C₂ to C₅ alkyl substituted with —OH, —COH, —COH, —COC, —NHOH, —CONHOH, or —COOH.

13. The compound of claim 8, wherein R₁ is selected from the group consisting of:

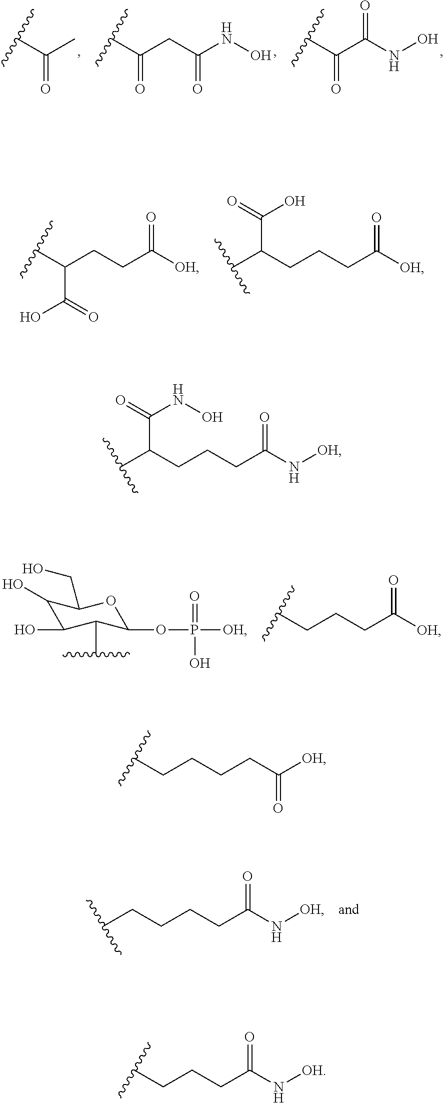

14. The compound of claim 7, wherein the compound of Formula (I) has a structure in accordance with Formula (III):

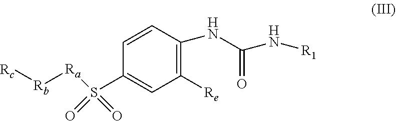

15. The compound of claim 14, wherein R₁ is a C₁ to C₁₀ alkyl substituted with —OH, —COH, —COH, —COC, —NHOH, —CONHOH, —COOH, or a combination thereof.

16. The compound of claim 14, wherein R₁ is selected from the group consisting of:

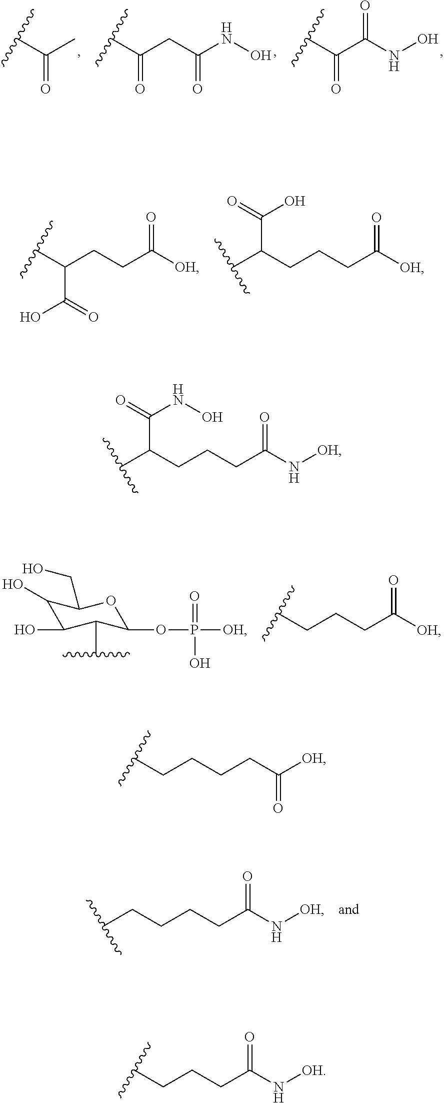

17. The compound of claim 1, wherein Rₐ is

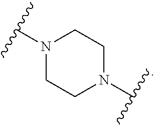 [SMILES: *N1CCN(*)CC1]

18. The compound of claim 1, wherein
R_{b} is selected from the group consisting of C₁ to C₁₀ unsubstituted or substituted alkyl; and
R_{c} comprises halogen, —CO₂CH₃, —COOH, —CN₂CF₃, —CF₃, —C₂OH, —CCOH, C₄ to C₁₀ unsubstituted or substituted aryl, unsubstituted or substituted four to ten member heterocycle ring, or C₁ to C₁₀ unsubstituted or substituted alkyl.

19. The compound of claim 18, wherein
R_{b} is selected from the group consisting of C₁ to C₄ unsubstituted or substituted alkyl; and
R_{c} comprises halogen, —CO₂CH₃, —COOH, —CN₂CF₃, —CF₃, —C₂OH, —CCOH, C₄ to C₁₀ unsubstituted or substituted aryl, unsubstituted or substituted four to ten member heterocycle ring, or C₁ to C₁₀ unsubstituted or substituted alkyl.

20. The compound of claim 1, wherein
R_{b} is selected from the group consisting of C₄ to C₁₀ unsubstituted or substituted aryl, and unsubstituted or substituted four to ten member heterocycle ring; and
R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, or C₁ to C₁₀ unsubstituted or substituted alkyl.

21. The compound of claim 20, wherein R_{c} comprises hydrogen, halogen, —OH, —CO₂CH₃, —COOH, —CN₂CF₃, —CF₃, —C₂OH, —CONHOH, —CCOH, or C₁ to C₁₀ unsubstituted or substituted alkyl.

22. The compound of claim 21, wherein R_{c} comprises two or more of hydrogen, halogen, —OH, —CO₂CH₃, —COOH, —CN₂CF₃, —CF₃, —C₂OH, —CONHOH, —CCOH, C₁ to C₁₀ unsubstituted or substituted alkyl, or a combination thereof.

23. The compound of claim 1, wherein the compound of Formula (I) has a structure in accordance with one of:

| Compound | |
|---|---|
| No. | Structure |
| 1 | 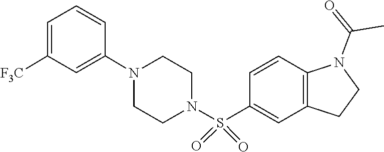 [SMILES: CC(=O)N1CCc2cc(S(=O)(=O)N3CCN(c4cccc(C(F)(F)F)c4)CC3)ccc21] |
| 2 | 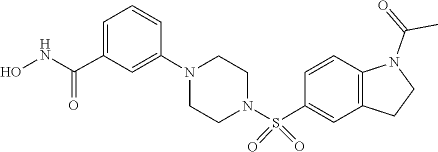 [SMILES: CC(=O)N1CCc2cc(S(=O)(=O)N3CCN(c4cccc(C(=O)NO)c4)CC3)ccc21] |
| 3 | 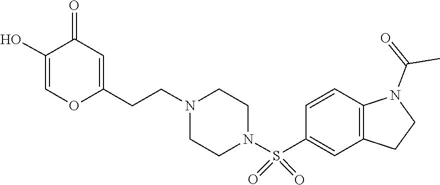 [SMILES: CC(=O)N1CCc2cc(S(=O)(=O)N3CCN(CCc4cc(=O)c(O)co4)CC3)ccc21] |
| 4 | 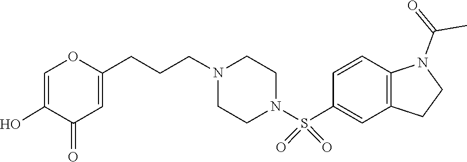 [SMILES: CC(=O)N1CCc2cc(S(=O)(=O)N3CCN(CCCc4cc(=O)c(O)co4)CC3)ccc21] |
| 5 | 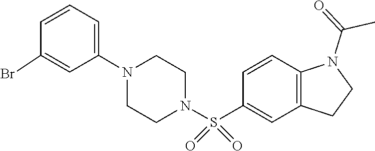 [SMILES: CC(=O)N1CCc2cc(S(=O)(=O)N3CCN(c4cccc(Br)c4)CC3)ccc21] |
| 6 | 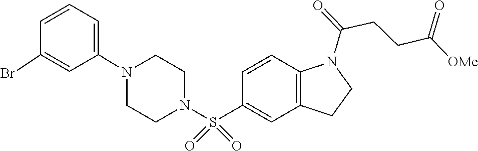 [SMILES: COC(=O)CCC(=O)N1CCc2cc(S(=O)(=O)N3CCN(c4cccc(Br)c4)CC3)ccc21] |
| 7 | 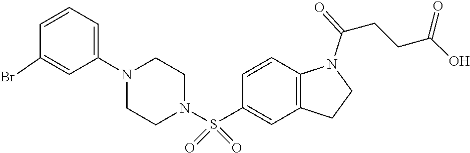 [SMILES: O=C(O)CCC(=O)N1CCc2cc(S(=O)(=O)N3CCN(c4cccc(Br)c4)CC3)ccc21] |
| 8 | 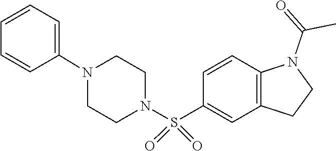 [SMILES: CC(=O)N1CCc2cc(S(=O)(=O)N3CCN(c4ccccc4)CC3)ccc21] |
| 9 | 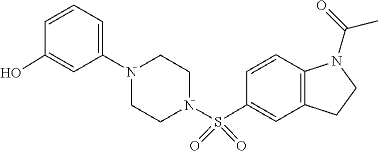 [SMILES: CC(=O)N1CCc2cc(S(=O)(=O)N3CCN(c4cccc(O)c4)CC3)ccc21] |
| 10 | 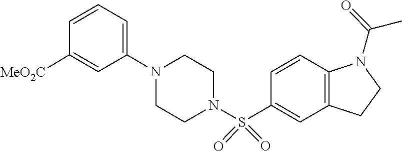 [SMILES: COC(=O)c1cccc(N2CCN(S(=O)(=O)c3ccc4c(c3)CCN4C(C)=O)CC2)c1] |
| 11 | 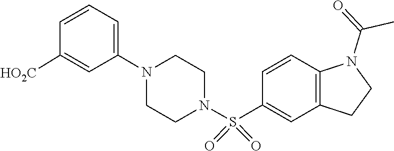 [SMILES: CC(=O)N1CCc2cc(S(=O)(=O)N3CCN(c4cccc(C(=O)O)c4)CC3)ccc21] |
| 12 | 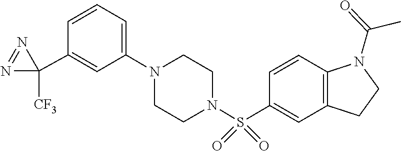 [SMILES: CC(=O)N1CCc2cc(S(=O)(=O)N3CCN(c4cccc(C5(C(F)(F)F)N=N5)c4)CC3)ccc21] |
| 13 | 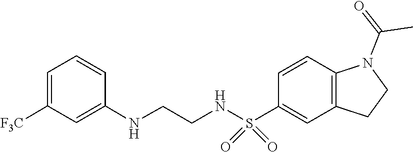 [SMILES: CC(=O)N1CCc2cc(S(=O)(=O)NCCNc3cccc(C(F)(F)F)c3)ccc21] |
| 14 | 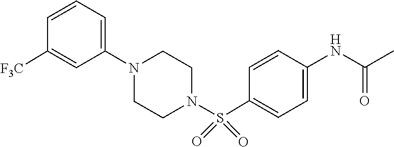 [SMILES: CC(=O)Nc1ccc(S(=O)(=O)N2CCN(c3cccc(C(F)(F)F)c3)CC2)cc1] |
| 15 | 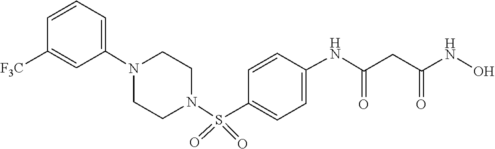 [SMILES: O=C(CC(=O)Nc1ccc(S(=O)(=O)N2CCN(c3cccc(C(F)(F)F)c3)CC2)cc1)NO] |
| 16 | 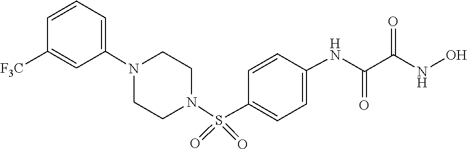 [SMILES: O=C(NO)C(=O)Nc1ccc(S(=O)(=O)N2CCN(c3cccc(C(F)(F)F)c3)CC2)cc1] |
| 17 | 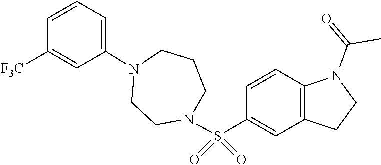 [SMILES: CC(=O)N1CCc2cc(S(=O)(=O)N3CCCN(c4cccc(C(F)(F)F)c4)CC3)ccc21] |
| 18 | 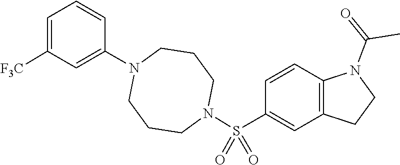 [SMILES: CC(=O)N1CCc2cc(S(=O)(=O)N3CCCN(c4cccc(C(F)(F)F)c4)CCC3)ccc21] |
| 19 | 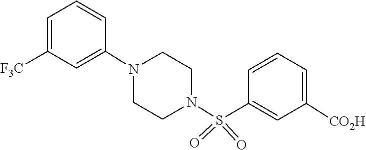 [SMILES: O=C(O)c1cccc(S(=O)(=O)N2CCN(c3cccc(C(F)(F)F)c3)CC2)c1] |
| 20 | 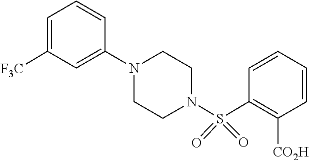 [SMILES: O=C(O)c1ccccc1S(=O)(=O)N1CCN(c2cccc(C(F)(F)F)c2)CC1] |
| 21 | 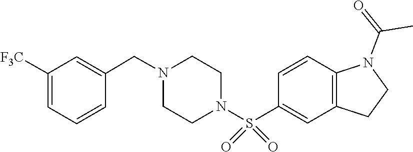 [SMILES: CC(=O)N1CCc2cc(S(=O)(=O)N3CCN(Cc4cccc(C(F)(F)F)c4)CC3)ccc21] |
| 22 | 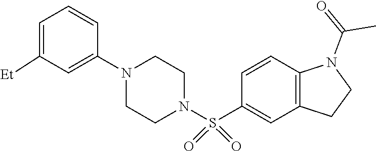 [SMILES: CCc1cccc(N2CCN(S(=O)(=O)c3ccc4c(c3)CCN4C(C)=O)CC2)c1] |
| 23 | 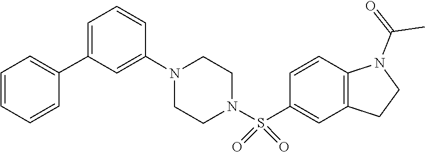 [SMILES: CC(=O)N1CCc2cc(S(=O)(=O)N3CCN(c4cccc(-c5ccccc5)c4)CC3)ccc21] |
| 24 | 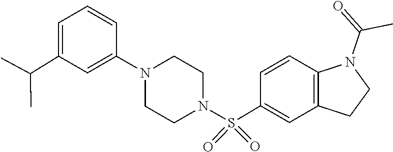 [SMILES: CC(=O)N1CCc2cc(S(=O)(=O)N3CCN(c4cccc(C(C)C)c4)CC3)ccc21] |
| 25 | 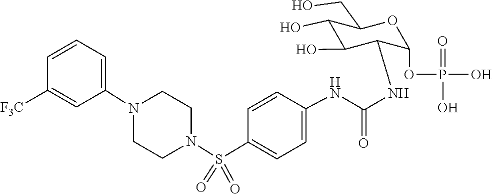 [SMILES: O=C(Nc1ccc(S(=O)(=O)N2CCN(c3cccc(C(F)(F)F)c3)CC2)cc1)NC1C(OP(=O)(O)O)OC(CO)C(O)C1O] |
| 26 | 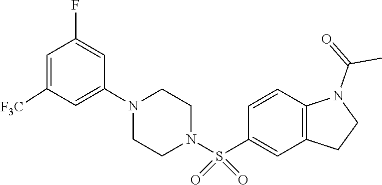 [SMILES: CC(=O)N1CCc2cc(S(=O)(=O)N3CCN(c4cc(F)cc(C(F)(F)F)c4)CC3)ccc21] |
| 27 | 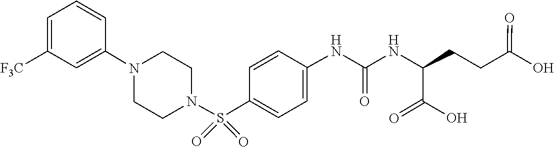 [SMILES: O=C(O)CC[C@H](NC(=O)Nc1ccc(S(=O)(=O)N2CCN(c3cccc(C(F)(F)F)c3)CC2)cc1)C(=O)O] |
| 28 | 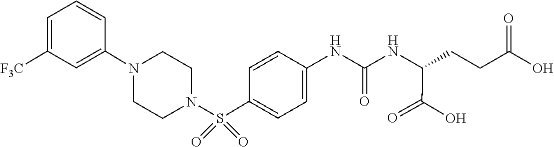 [SMILES: O=C(O)CC[C@@H](NC(=O)Nc1ccc(S(=O)(=O)N2CCN(c3cccc(C(F)(F)F)c3)CC2)cc1)C(=O)O] |
| 29 | 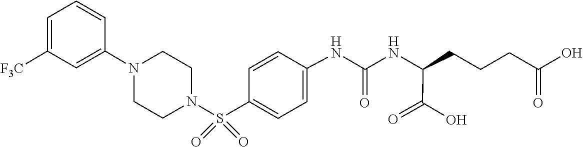 [SMILES: O=C(O)CCC[C@H](NC(=O)Nc1ccc(S(=O)(=O)N2CCN(c3cccc(C(F)(F)F)c3)CC2)cc1)C(=O)O] |
| 30 | 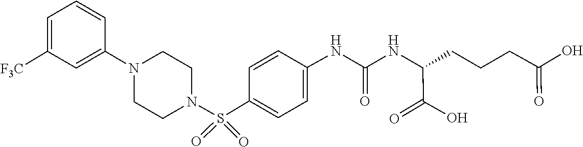 [SMILES: O=C(O)CCC[C@@H](NC(=O)Nc1ccc(S(=O)(=O)N2CCN(c3cccc(C(F)(F)F)c3)CC2)cc1)C(=O)O] |
| 31 | 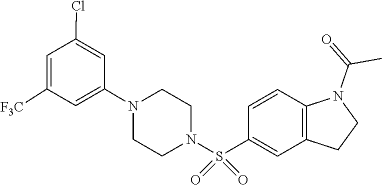 [SMILES: CC(=O)N1CCc2cc(S(=O)(=O)N3CCN(c4cc(Cl)cc(C(F)(F)F)c4)CC3)ccc21] |
| 32 | 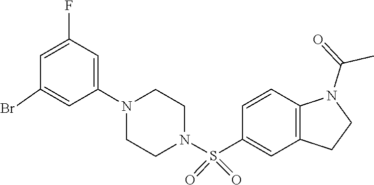 [SMILES: CC(=O)N1CCc2cc(S(=O)(=O)N3CCN(c4cc(F)cc(Br)c4)CC3)ccc21] |
| 33 | 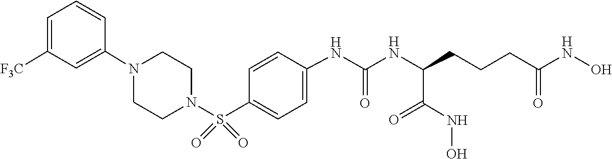 [SMILES: O=C(CCC[C@H](NC(=O)Nc1ccc(S(=O)(=O)N2CCN(c3cccc(C(F)(F)F)c3)CC2)cc1)C(=O)NO)NO] |
| 34 | 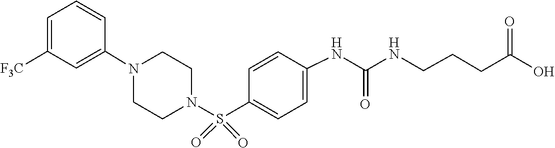 [SMILES: O=C(O)CCCNC(=O)Nc1ccc(S(=O)(=O)N2CCN(c3cccc(C(F)(F)F)c3)CC2)cc1] |
| 35 | 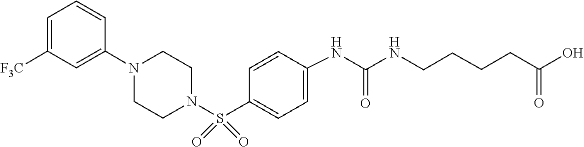 [SMILES: O=C(O)CCCCNC(=O)Nc1ccc(S(=O)(=O)N2CCN(c3cccc(C(F)(F)F)c3)CC2)cc1] |
| 36 | 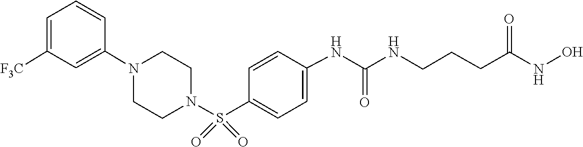 [SMILES: O=C(CCCNC(=O)Nc1ccc(S(=O)(=O)N2CCN(c3cccc(C(F)(F)F)c3)CC2)cc1)NO] |
| 37 | 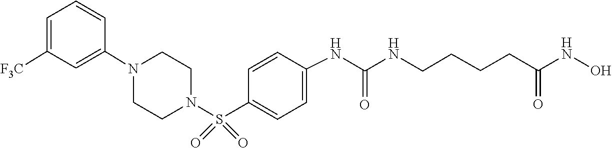 [SMILES: O=C(CCCCNC(=O)Nc1ccc(S(=O)(=O)N2CCN(c3cccc(C(F)(F)F)c3)CC2)cc1)NO] |
| 38 | 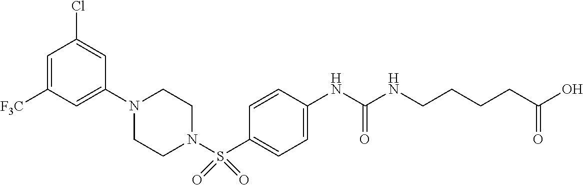 [SMILES: O=C(O)CCCCNC(=O)Nc1ccc(S(=O)(=O)N2CCN(c3cc(Cl)cc(C(F)(F)F)c3)CC2)cc1] |
| 39 | 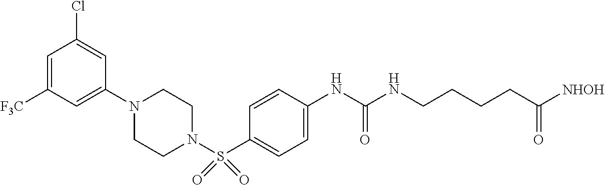 |
| 40 | 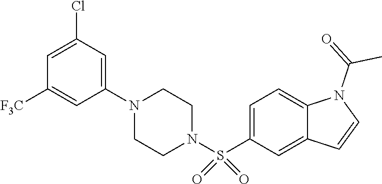 [SMILES: CC(=O)n1ccc2cc(S(=O)(=O)N3CCN(c4cc(Cl)cc(C(F)(F)F)c4)CC3)ccc21] |
| 41 | 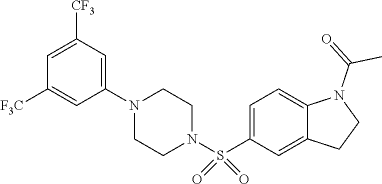 [SMILES: CC(=O)N1CCc2cc(S(=O)(=O)N3CCN(c4cc(C(F)(F)F)cc(C(F)(F)F)c4)CC3)ccc21] |
| 42 | 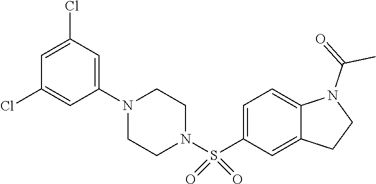 [SMILES: CC(=O)N1CCc2cc(S(=O)(=O)N3CCN(c4cc(Cl)cc(Cl)c4)CC3)ccc21] |

24. A composition comprising:
(a) an amount of a compound of Formula (I) or a salt thereof: 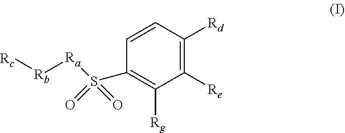
wherein
Rₐ is selected from the group consisting of: 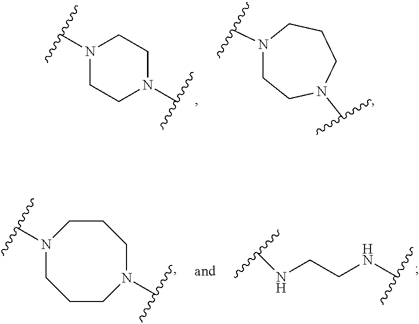 [SMILES: *N1CCN(*)CC1 ; *N1CCCN(*)CC1 ; *N1CCCN(*)CCC1 ; *NCCN*]
R_{b} is selected from the group consisting of single bond, C₄ to C₁₀ unsubstituted or substituted aryl, unsubstituted or substituted four to ten member heterocycle ring, and C₁ to C₁₀ unsubstituted or substituted alkyl;
R_{c} comprises hydrogen, halogen, —OH, —CO₂CH₃, —COOH, —CN₂CF₃, —CF₃, —C₂OH, —CONHOH, —CCOH, C₄ to C₁₀ unsubstituted or substituted aryl, unsubstituted or substituted four to ten member heterocycle ring, or C₁ to C₁₀ unsubstituted or substituted alkyl;
R_{d} and Rₑ are independently hydrogen, —OH, —COH, —COH, —COC, —COOH, R_{f}, or are taken together as an unsubstituted or substituted four to eight member nitrogen containing heterocycle ring,
wherein R_{f} is selected from the group consisting of: 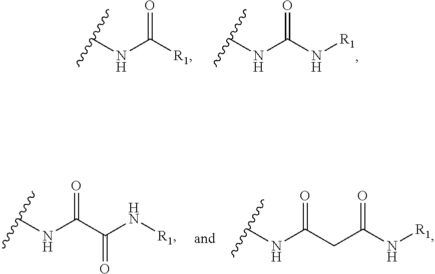
R₁ is hydrogen, methyl, —OH, C₁ to C₁₀ alkyl unsubstituted or substituted with —OH, —COH, —COH, —COC, —NHOH, —CONHOH, —COOH, an unsubstituted or substituted four to eight member heterocycle ring, or a combination thereof; and;
R_{g} is hydrogen, —OH, —COH, —COH, —COC, —COOH, or C₁ to C₅ unsubstituted or substituted alkyl; and
(b) a pharmaceutical expedient.

25. The composition of claim 24, wherein the composition comprises a therapeutically effective amount of the compound of Formula (I) or a salt thereof.

26. The composition of claim 25, wherein the therapeutically effective amount of the compound comprising Formula (I) or a salt thereof is more than about 1 □g.

27. A method of treating a Gram-negative bacterial infection in a patient, the method comprising: administering a composition of claim 24 to a patient in need of treatment thereof.
